SEQUENCE LISTING (136 sequences)

SEQ ID NO: 1 - DNA, 25 bp, synthetic construct
  misc_feature 1..25: note = Synthetic Oligonucleotide
  source 1..25: mol_type = other DNA
aaaatgcggttgccacttaaacccc

SEQ ID NO: 2 - DNA, 18 bp, synthetic construct
  misc_feature 1..18: note = Synthetic Oligonucleotide
  source 1..18: mol_type = other DNA
aacaaccggccagcgtgg

SEQ ID NO: 3 - DNA, 27 bp, synthetic construct
  misc_feature 1..27: note = Synthetic Oligonucleotide
  source 1..27: mol_type = other DNA
aacactttatcgctactgtcattaggt

SEQ ID NO: 4 - DNA, 28 bp, synthetic construct
  misc_feature 1..28: note = Synthetic Oligonucleotide
  source 1..28: mol_type = other DNA
aaccctgtttcaccatcataaaggtcgc

SEQ ID NO: 5 - DNA, 20 bp, synthetic construct
  misc_feature 1..20: note = Synthetic Oligonucleotide
  source 1..20: mol_type = other DNA
aagattatcgacgccgcacg

SEQ ID NO: 6 - DNA, 28 bp, synthetic construct
  misc_feature 1..28: note = Synthetic Oligonucleotide
  source 1..28: mol_type = other DNA
aagcattttatcaatcaggttcattccg

SEQ ID NO: 7 - DNA, 26 bp, synthetic construct
  misc_feature 1..26: note = Synthetic Oligonucleotide
  source 1..26: mol_type = other DNA
acatccatgtgatatggaacaaattg

SEQ ID NO: 8 - DNA, 23 bp, synthetic construct
  misc_feature 1..23: note = Synthetic Oligonucleotide
  source 1..23: mol_type = other DNA
accggctgagcctcagactcagc

SEQ ID NO: 9 - DNA, 27 bp, synthetic construct
  misc_feature 1..27: note = Synthetic Oligonucleotide
  source 1..27: mol_type = other DNA
acgacttcttagccgagcacccaagcg

SEQ ID NO: 10 - DNA, 24 bp, synthetic construct
  misc_feature 1..24: note = Synthetic Oligonucleotide
  source 1..24: mol_type = other DNA
acggttttatggttaagactcccg

SEQ ID NO: 11 - DNA, 23 bp, synthetic construct
  misc_feature 1..23: note = Synthetic Oligonucleotide
  source 1..23: mol_type = other DNA
actttatcgctactgtcattagg

SEQ ID NO: 12 - DNA, 23 bp, synthetic construct
  misc_feature 1..23: note = Synthetic Oligonucleotide
  source 1..23: mol_type = other DNA
agaaggtgactttattgtgcgcg

SEQ ID NO: 13 - DNA, 22 bp, synthetic construct
  misc_feature 1..22: note = Synthetic Oligonucleotide
  source 1..22: mol_type = other DNA
agcgcaccattactggtggtta

SEQ ID NO: 14 - DNA, 27 bp, synthetic construct
  misc_feature 1..27: note = Synthetic Oligonucleotide
  source 1..27: mol_type = other DNA
agcggaatcacaagtcaatcatcgcgc

SEQ ID NO: 15 - DNA, 24 bp, synthetic construct
  misc_feature 1..24: note = Synthetic Oligonucleotide
  source 1..24: mol_type = other DNA
agcggttgaatcggcgatgggtga

SEQ ID NO: 16 - DNA, 25 bp, synthetic construct
  misc_feature 1..25: note = Synthetic Oligonucleotide
  source 1..25: mol_type = other DNA
agctattatgtgtgtccatttatac

SEQ ID NO: 17 - DNA, 24 bp, synthetic construct
  misc_feature 1..24: note = Synthetic Oligonucleotide
  source 1..24: mol_type = other DNA
aggcacaaattcagcaacaacaac

SEQ ID NO: 18 - DNA, 27 bp, synthetic construct
  misc_feature 1..27: note = Synthetic Oligonucleotide
  source 1..27: mol_type = other DNA
agggcagttcagtgatgcattatcgac

SEQ ID NO: 19 - DNA, 21 bp, synthetic construct
  misc_feature 1..21: note = Synthetic Oligonucleotide
  source 1..21: mol_type = other DNA
ataagcagtcgattccccaag

SEQ ID NO: 20 - DNA, 19 bp, synthetic construct
  misc_feature 1..19: note = Synthetic Oligonucleotide
  source 1..19: mol_type = other DNA
atcttcgcttcgcttgggt

SEQ ID NO: 21 - DNA, 20 bp, synthetic construct
  misc_feature 1..20: note = Synthetic Oligonucleotide
  source 1..20: mol_type = other DNA
atcttcgcttcgcttgggtt

SEQ ID NO: 22 - DNA, 18 bp, synthetic construct
  misc_feature 1..18: note = Synthetic Oligonucleotide
  source 1..18: mol_type = other DNA
atgatggtgcgcttgctg

SEQ ID NO: 23 - DNA, 20 bp, synthetic construct
  misc_feature 1..20: note = Synthetic Oligonucleotide
  source 1..20: mol_type = other DNA
atggtcaattagagacaaag

SEQ ID NO: 24 - DNA, 24 bp, synthetic construct
  misc_feature 1..24: note = Synthetic Oligonucleotide
  source 1..24: mol_type = other DNA
atgtgaccgagttcaacgtcaggg

SEQ ID NO: 25 - DNA, 28 bp, synthetic construct
  misc_feature 1..28: note = Synthetic Oligonucleotide
  source 1..28: mol_type = other DNA
caagagaactttctttgagggagaattt

SEQ ID NO: 26 - DNA, 19 bp, synthetic construct
  misc_feature 1..19: note = Synthetic Oligonucleotide
  source 1..19: mol_type = other DNA
cacggcaaatcgtacatca

SEQ ID NO: 27 - DNA, 24 bp, synthetic construct
  misc_feature 1..24: note = Synthetic Oligonucleotide
  source 1..24: mol_type = other DNA
ccaacaaagttggatgtatggctt

SEQ ID NO: 28 - DNA, 21 bp, synthetic construct
  misc_feature 1..21: note = Synthetic Oligonucleotide
  source 1..21: mol_type = other DNA
ccacgccaatacgatgattgt

SEQ ID NO: 29 - DNA, 26 bp, synthetic construct
  misc_feature 1..26: note = Synthetic Oligonucleotide
  source 1..26: mol_type = other DNA
cccatcacggtgaaatttacagtgac

SEQ ID NO: 30 - DNA, 19 bp, synthetic construct
  misc_feature 1..19: note = Synthetic Oligonucleotide
  source 1..19: mol_type = other DNA
cccttaatatcggcgctgg

SEQ ID NO: 31 - DNA, 27 bp, synthetic construct
  misc_feature 1..27: note = Synthetic Oligonucleotide
  source 1..27: mol_type = other DNA
ccgcagtggaatccttcatggcagaca

SEQ ID NO: 32 - DNA, 20 bp, synthetic construct
  misc_feature 1..20: note = Synthetic Oligonucleotide
  source 1..20: mol_type = other DNA
ccgtacttgcagccctaagc

SEQ ID NO: 33 - DNA, 25 bp, synthetic construct
  misc_feature 1..25: note = Synthetic Oligonucleotide
  source 1..25: mol_type = other DNA
cctctagcggttgtgggtgatacgg

SEQ ID NO: 34 - DNA, 27 bp, synthetic construct
  misc_feature 1..27: note = Synthetic Oligonucleotide
  source 1..27: mol_type = other DNA
cgaaatcacgagattgatgccaaccat

SEQ ID NO: 35 - DNA, 20 bp, synthetic construct
  misc_feature 1..20: note = Synthetic Oligonucleotide
  source 1..20: mol_type = other DNA
cgcgcgggtattgcctcggt

SEQ ID NO: 36 - DNA, 26 bp, synthetic construct
  misc_feature 1..26: note = Synthetic Oligonucleotide
  source 1..26: mol_type = other DNA
cggaatcacaagtcaatcatcgcgca

SEQ ID NO: 37 - DNA, 20 bp, synthetic construct
  misc_feature 1..20: note = Synthetic Oligonucleotide
  source 1..20: mol_type = other DNA
cgtgcggcgtcgataatctt

SEQ ID NO: 38 - DNA, 20 bp, synthetic construct
  misc_feature 1..20: note = Synthetic Oligonucleotide
  source 1..20: mol_type = other DNA
cgttcactattggtctctgc

SEQ ID NO: 39 - DNA, 21 bp, synthetic construct
  misc_feature 1..21: note = Synthetic Oligonucleotide
  source 1..21: mol_type = other DNA
cgttcactattggtctctgca

SEQ ID NO: 40 - DNA, 27 bp, synthetic construct
  misc_feature 1..27: note = Synthetic Oligonucleotide
  source 1..27: mol_type = other DNA
ctgttgcccctgaatatctaaagtgac

SEQ ID NO: 41 - DNA, 23 bp, synthetic construct
  misc_feature 1..23: note = Synthetic Oligonucleotide
  source 1..23: mol_type = other DNA
gaagcctggactgattatttaca

SEQ ID NO: 42 - DNA, 20 bp, synthetic construct
  misc_feature 1..20: note = Synthetic Oligonucleotide
  source 1..20: mol_type = other DNA
gcagtggaatccttcatggc

SEQ ID NO: 43 - DNA, 22 bp, synthetic construct
  misc_feature 1..22: note = Synthetic Oligonucleotide
  source 1..22: mol_type = other DNA
gccaaaccaaagacaaactggt

SEQ ID NO: 44 - DNA, 25 bp, synthetic construct
  misc_feature 1..25: note = Synthetic Oligonucleotide
  source 1..25: mol_type = other DNA
gctattatgtgtgtccatttatacg

SEQ ID NO: 45 - DNA, 25 bp, synthetic construct
  misc_feature 1..25: note = Synthetic Oligonucleotide
  source 1..25: mol_type = other DNA
ggcaaatcaggaagtaaaacactgg

SEQ ID NO: 46 - DNA, 24 bp, synthetic construct
  misc_feature 1..24: note = Synthetic Oligonucleotide
  source 1..24: mol_type = other DNA
gggtgtgataagcattgcattaac

SEQ ID NO: 47 - DNA, 21 bp, synthetic construct
  misc_feature 1..21: note = Synthetic Oligonucleotide
  source 1..21: mol_type = other DNA
gtgcggcgtcgataatcttag

SEQ ID NO: 48 - DNA, 23 bp, synthetic construct
  misc_feature 1..23: note = Synthetic Oligonucleotide
  source 1..23: mol_type = other DNA
tacattcctaacgtggatgtcag

SEQ ID NO: 49 - DNA, 23 bp, synthetic construct
  misc_feature 1..23: note = Synthetic Oligonucleotide
  source 1..23: mol_type = other DNA
tactgacgggctttgcgcattag

SEQ ID NO: 50 - DNA, 28 bp, synthetic construct
  misc_feature 1..28: note = Synthetic Oligonucleotide
  source 1..28: mol_type = other DNA
tcattgcgtagggccagcgttattctgc

SEQ ID NO: 51 - DNA, 18 bp, synthetic construct
  misc_feature 1..18: note = Synthetic Oligonucleotide
  source 1..18: mol_type = other DNA
tccacacgcaaacttacc

SEQ ID NO: 52 - DNA, 19 bp, synthetic construct
  misc_feature 1..19: note = Synthetic Oligonucleotide
  source 1..19: mol_type = other DNA
tccacacgcaaacttaccg

SEQ ID NO: 53 - DNA, 20 bp, synthetic construct
  misc_feature 1..20: note = Synthetic Oligonucleotide
  source 1..20: mol_type = other DNA
tccacacgcaaacttaccgt

SEQ ID NO: 54 - DNA, 26 bp, synthetic construct
  misc_feature 1..26: note = Synthetic Oligonucleotide
  source 1..26: mol_type = other DNA
tcccaatcactactgacttcggctgg

SEQ ID NO: 55 - DNA, 20 bp, synthetic construct
  misc_feature 1..20: note = Synthetic Oligonucleotide
  source 1..20: mol_type = other DNA
tccgcagtggaatccttcat

SEQ ID NO: 56 - DNA, 20 bp, synthetic construct
  misc_feature 1..20: note = Synthetic Oligonucleotide
  source 1..20: mol_type = other DNA
tccgctcctgtatttgctca

SEQ ID NO: 57 - DNA, 21 bp, synthetic construct
  misc_feature 1..21: note = Synthetic Oligonucleotide
  source 1..21: mol_type = other DNA
tccgctcctgtatttgctcac

SEQ ID NO: 58 - DNA, 22 bp, synthetic construct
  misc_feature 1..22: note = Synthetic Oligonucleotide
  source 1..22: mol_type = other DNA
tcgagccaaaccaaagacaaac

SEQ ID NO: 59 - DNA, 21 bp, synthetic construct
  misc_feature 1..21: note = Synthetic Oligonucleotide
  source 1..21: mol_type = other DNA
tctacttttggccagttctac

SEQ ID NO: 60 - DNA, 21 bp, synthetic construct
  misc_feature 1..21: note = Synthetic Oligonucleotide
  source 1..21: mol_type = other DNA
tgaagcagagtcataccaagg

SEQ ID NO: 61 - DNA, 23 bp, synthetic construct
  misc_feature 1..23: note = Synthetic Oligonucleotide
  source 1..23: mol_type = other DNA
tgacgggctttgcgcattagcgc

SEQ ID NO: 62 - DNA, 20 bp, synthetic construct
  misc_feature 1..20: note = Synthetic Oligonucleotide
  source 1..20: mol_type = other DNA
tgcggcgtcgataatcttag

SEQ ID NO: 63 - DNA, 29 bp, synthetic construct
  misc_feature 1..29: note = Synthetic Oligonucleotide
  source 1..29: mol_type = other DNA
tgcgtatccatctgccaggcaacgacttc

SEQ ID NO: 64 - DNA, 26 bp, synthetic construct
  misc_feature 1..26: note = Synthetic Oligonucleotide
  source 1..26: mol_type = other DNA
tgctattaactgccaactcactttga

SEQ ID NO: 65 - DNA, 23 bp, synthetic construct
  misc_feature 1..23: note = Synthetic Oligonucleotide
  source 1..23: mol_type = other DNA
tgctgtcttcatttggagcattc

SEQ ID NO: 66 - DNA, 20 bp, synthetic construct
  misc_feature 1..20: note = Synthetic Oligonucleotide
  source 1..20: mol_type = other DNA
tggaatccttcatggcagac

SEQ ID NO: 67 - DNA, 23 bp, synthetic construct
  misc_feature 1..23: note = Synthetic Oligonucleotide
  source 1..23: mol_type = other DNA
tggagccgatttattcgccacga

SEQ ID NO: 68 - DNA, 25 bp, synthetic construct
  misc_feature 1..25: note = Synthetic Oligonucleotide
  source 1..25: mol_type = other DNA
tggccggttgttgttgctgaatttg

SEQ ID NO: 69 - DNA, 23 bp, synthetic construct
  misc_feature 1..23: note = Synthetic Oligonucleotide
  source 1..23: mol_type = other DNA
tgggacatggtcaattagagaca

SEQ ID NO: 70 - DNA, 21 bp, synthetic construct
  misc_feature 1..21: note = Synthetic Oligonucleotide
  source 1..21: mol_type = other DNA
ttcagcgcaatcttcaattac

SEQ ID NO: 71 - DNA, 24 bp, synthetic construct
  misc_feature 1..24: note = Synthetic Oligonucleotide
  source 1..24: mol_type = other DNA
ttcagcgcaatcttcaattacaaa

SEQ ID NO: 72 - DNA, 28 bp, synthetic construct
  misc_feature 1..28: note = Synthetic Oligonucleotide
  source 1..28: mol_type = other DNA
ttcctaacgtggatgtcagtggcggtcc

SEQ ID NO: 73 - DNA, 22 bp, synthetic construct
  misc_feature 1..22: note = Synthetic Oligonucleotide
  source 1..22: mol_type = other DNA
ttctacttttggccagttctac

SEQ ID NO: 74 - DNA, 25 bp, synthetic construct
  misc_feature 1..25: note = Synthetic Oligonucleotide
  source 1..25: mol_type = other DNA
ttctacttttggccagttctacaat

SEQ ID NO: 75 - DNA, 20 bp, synthetic construct
  misc_feature 1..20: note = Synthetic Oligonucleotide
  source 1..20: mol_type = other DNA
ttctgcacttggccagaaac

SEQ ID NO: 76 - DNA, 22 bp, synthetic construct
  misc_feature 1..22: note = Synthetic Oligonucleotide
  source 1..22: mol_type = other DNA
ttctgcacttggccagaaacga

SEQ ID NO: 77 - DNA, 19 bp, synthetic construct
  misc_feature 1..19: note = Synthetic Oligonucleotide
  source 1..19: mol_type = other DNA
ttgccgggcgctcattata

SEQ ID NO: 78 - DNA, 20 bp, synthetic construct
  misc_feature 1..20: note = Synthetic Oligonucleotide
  source 1..20: mol_type = other DNA
ttgggtgctcggctaagaag

SEQ ID NO: 79 - DNA, 19 bp, synthetic construct
  misc_feature 1..19: note = Synthetic Oligonucleotide
  source 1..19: mol_type = other DNA
tttcatttgccgccaacgc

SEQ ID NO: 80 - DNA, 30 bp, synthetic construct
  misc_feature 1..30: note = Synthetic Oligonucleotide
  source 1..30: mol_type = other DNA
ttcctaacgtggatgtcagtggcggtccgc

SEQ ID NO: 81 - DNA, 27 bp, synthetic construct
  misc_feature 1..27: note = Synthetic Oligonucleotide
  source 1..27: mol_type = other DNA
tggccggttgttgttgctgaatttgtg

SEQ ID NO: 82 - DNA, 24 bp, synthetic construct
  misc_feature 1..24: note = Synthetic Oligonucleotide
  source 1..24: mol_type = other DNA
tggagccgatttattcgccacgac

SEQ ID NO: 83 - DNA, 31 bp, synthetic construct
  misc_feature 1..31: note = Synthetic Oligonucleotide
  source 1..31: mol_type = other DNA
tgcgtatccatctgccaggcaacgacttcgc

SEQ ID NO: 84 - DNA, 19 bp, synthetic construct
  misc_feature 1..19: note = Synthetic Oligonucleotide
  source 1..19: mol_type = other DNA
gtgcggcgtcgataatctt

SEQ ID NO: 85 - DNA, 18 bp, synthetic construct
  misc_feature 1..18: note = Synthetic Oligonucleotide
  source 1..18: mol_type = other DNA
gtacttgcagccctaagc

SEQ ID NO: 86 - DNA, 20 bp, synthetic construct
  misc_feature 1..20: note = Synthetic Oligonucleotide
  source 1..20: mol_type = other DNA
gcttcgcttgggttttcgac

SEQ ID NO: 87 - DNA, 24 bp, synthetic construct
  misc_feature 1..24: note = Synthetic Oligonucleotide
  source 1..24: mol_type = other DNA
gctattatgtgtgtccatttatac

SEQ ID NO: 88 - DNA, 21 bp, synthetic construct
  misc_feature 1..21: note = Synthetic Oligonucleotide
  source 1..21: mol_type = other DNA
gcgcgcgggtattgcctcggt

SEQ ID NO: 89 - DNA, 26 bp, synthetic construct
  misc_feature 1..26: note = Synthetic Oligonucleotide
  source 1..26: mol_type = other DNA
gcctctagcggttgtgggtgatacgg

SEQ ID NO: 90 - DNA, 21 bp, synthetic construct
  misc_feature 1..21: note = Synthetic Oligonucleotide
  source 1..21: mol_type = other DNA
cttgctcattcsataggctgg

SEQ ID NO: 91 - DNA, 24 bp, synthetic construct
  misc_feature 1..24: note = Synthetic Oligonucleotide
  source 1..24: mol_type = other DNA
ctctagcggttgtgggtgatacgg

SEQ ID NO: 92 - DNA, 19 bp, synthetic construct
  misc_feature 1..19: note = Synthetic Oligonucleotide
  source 1..19: mol_type = other DNA
cgtacttgcagccctaagc

SEQ ID NO: 93 - DNA, 20 bp, synthetic construct
  misc_feature 1..20: note = Synthetic Oligonucleotide
  source 1..20: mol_type = other DNA
ccgagtacattggcatcgtg

SEQ ID NO: 94 - DNA, 20 bp, synthetic construct
  misc_feature 1..20: note = Synthetic Oligonucleotide
  source 1..20: mol_type = other DNA
atacgcagagtcaagcagtg

SEQ ID NO: 95 - DNA, 27 bp, synthetic construct
  misc_feature 1..27: note = Synthetic Oligonucleotide
  source 1..27: mol_type = other DNA
aggcacaaattcagcaacaacaaccgg

SEQ ID NO: 96 - DNA, 27 bp, synthetic construct
  misc_feature 1..27: note = Synthetic Oligonucleotide
  source 1..27: mol_type = other DNA
actaggtagattaattccacgccaacc

SEQ ID NO: 97 - DNA, 25 bp, synthetic construct
  misc_feature 1..25: note = Synthetic Oligonucleotide
  source 1..25: mol_type = other DNA
acggttttatggttaagactcccgc

SEQ ID NO: 98 - DNA, 22 bp, synthetic construct
  misc_feature 1..22: note = Synthetic Oligonucleotide
  source 1..22: mol_type = other DNA
acctgcaaaatgcggttgccac

SEQ ID NO: 99 - DNA, 27 bp, synthetic construct
  misc_feature 1..27: note = Synthetic Oligonucleotide
  source 1..27: mol_type = other DNA
acagggtaaaaaactggccttgtttcg

SEQ ID NO: 100 - DNA, 21 bp, synthetic construct
  misc_feature 1..21: note = Synthetic Oligonucleotide
  source 1..21: mol_type = other DNA
acacgatgccaatgtactcgg

SEQ ID NO: 101 - DNA, 23 bp, synthetic construct
  misc_feature 1..23: note = Synthetic Oligonucleotide
  source 1..23: mol_type = other DNA
aattctaactaggaccgcagagg

SEQ ID NO: 102 - DNA, 29 bp, synthetic construct
  misc_feature 1..29: note = Synthetic Oligonucleotide
  source 1..29: mol_type = other DNA
aagcattttatcaatcaggttcattccgg

SEQ ID NO: 103 - DNA, 28 bp, synthetic construct
  misc_feature 1..28: note = Synthetic Oligonucleotide
  source 1..28: mol_type = other DNA
aagcaccctatagctgaggatcttggtt

SEQ ID NO: 104 - DNA, 27 bp, synthetic construct
  misc_feature 1..27: note = Synthetic Oligonucleotide
  source 1..27: mol_type = other DNA
aagagaggaattaaggaatcaccttgc

SEQ ID NO: 105 - DNA, 26 bp, synthetic construct
  misc_feature 1..26: note = Synthetic Oligonucleotide
  source 1..26: mol_type = other DNA
aacactttatcgctactgtcattagg

SEQ ID NO: 106 - DNA, 22 bp, synthetic construct
  misc_feature 1..22: note = Synthetic Oligonucleotide
  source 1..22: mol_type = other DNA
  modified_base 6: mod_base = m5c
  modified_base 21: mod_base = m5c
aaatgcggttgccacttaaacc

SEQ ID NO: 107 - DNA, 24 bp, synthetic construct
  misc_feature 1..24: note = Synthetic Oligonucleotide
  source 1..24: mol_type = other DNA
ctattatgtgtgtccatttatacg

SEQ ID NO: 108 - DNA, 22 bp, synthetic construct
  misc_feature 1..22: note = Synthetic Oligonucleotide
  source 1..22: mol_type = other DNA
  modified_base 2: mod_base = OTHER; note = propyne dU
  modified_base 10: mod_base = OTHER; note = propyne dU
ttctgcacttggccagaaacga

SEQ ID NO: 109 - DNA, 23 bp, synthetic construct
  misc_feature 1..23: note = Synthetic Oligonucleotide
  source 1..23: mol_type = other DNA
  modified_base 3..5: mod_base = OTHER; note = propyne dU
  modified_base 7: mod_base = OTHER; note = propyne dU
  modified_base 8: mod_base = m5c
  modified_base 10: mod_base = m5c
  modified_base 11: mod_base = OTHER; note = propyne dU
  modified_base 14: mod_base = OTHER; note = propyne dU
  modified_base 16: mod_base = OTHER; note = propyne dU
  modified_base 17: mod_base = m5c
  modified_base 19..20: mod_base = OTHER; note = propyne dU
actttatcgctactgtcattagg

SEQ ID NO: 110 - DNA, 20 bp, synthetic construct
  misc_feature 1..20: note = Synthetic Oligonucleotide
  source 1..20: mol_type = other DNA
  modified_base 6: mod_base = OTHER; note = propyne dU
  modified_base 10: mod_base = OTHER; note = propyne dU
tggaatccttcatggcagac

SEQ ID NO: 111 - DNA, 20 bp, synthetic construct
  misc_feature 1..20: note = Synthetic Oligonucleotide
  source 1..20: mol_type = other DNA
  modified_base 1: mod_base = OTHER; note = propyne dU
  modified_base 3: mod_base = m5c
  modified_base 6: mod_base = m5c
  modified_base 8: mod_base = OTHER; note = propyne dU
  modified_base 9: mod_base = m5c
  modified_base 12: mod_base = OTHER; note = propyne dU
  modified_base 15: mod_base = OTHER; note = propyne dU
  modified_base 16: mod_base = m5c
  modified_base 17..18: mod_base = OTHER; note = propyne dU
tgcggcgtcgataatcttag

SEQ ID NO: 112 - DNA, 21 bp, synthetic construct
  misc_feature 1..21: note = Synthetic Oligonucleotide
  source 1..21: mol_type = other DNA
  modified_base 3: mod_base = m5c
  modified_base 6: mod_base = m5c
  modified_base 8: mod_base = m5c
  modified_base 12: mod_base = m5c
  modified_base 15: mod_base = m5c
ttcagcgcaatcttcaattac

SEQ ID NO: 113 - DNA, 25 bp, synthetic construct
  misc_feature 1..25: note = Synthetic Oligonucleotide
  source 1..25: mol_type = other DNA
  modified_base 4: mod_base = OTHER; note = propyne dU
  modified_base 6..7: mod_base = OTHER; note = propyne dU
  modified_base 9: mod_base = OTHER; note = propyne dU
  modified_base 11: mod_base = OTHER; note = propyne dU
  modified_base 13: mod_base = OTHER; note = propyne dU
  modified_base 15: mod_base = OTHER; note = propyne dU
  modified_base 16: mod_base = m5c
  modified_base 19..21: mod_base = OTHER; note = propyne dU
  modified_base 23: mod_base = OTHER; note = propyne dU
agctattatgtgtgtccatttatac

SEQ ID NO: 114 - DNA, 27 bp, synthetic construct
  misc_feature 1..27: note = Synthetic Oligonucleotide
  source 1..27: mol_type = other DNA
  modified_base 7..10: mod_base = m5c
  modified_base 18: mod_base = m5c
ctgttgcccctgaatatctaaagtgac

SEQ ID NO: 115 - DNA, 23 bp, synthetic construct
  misc_feature 1..23: note = Synthetic Oligonucleotide
  source 1..23: mol_type = other DNA
  modified_base 7: mod_base = OTHER; note = propyne dU
  modified_base 13: mod_base = OTHER; note = propyne dU
  modified_base 15..16: mod_base = OTHER; note = propyne dU
  modified_base 18: mod_base = OTHER; note = propyne dU
  modified_base 20: mod_base = m5c
agaaggtgactttattgtgcgcg

SEQ ID NO: 116 - DNA, 22 bp, synthetic construct
  source 1..22: mol_type = other DNA
ttctacttttggccagttctac

SEQ ID NO: 117 - DNA, 23 bp, synthetic construct
  misc_feature 1..23: note = Synthetic Oligonucleotide
  source 1..23: mol_type = other DNA
  modified_base 3: mod_base = m5c
  modified_base 5..6: mod_base = OTHER; note = propyne dU
  modified_base 7..8: mod_base = m5c
  modified_base 9: mod_base = OTHER; note = propyne dU
  modified_base 12: mod_base = m5c
  modified_base 14: mod_base = OTHER; note = propyne dU
  modified_base 18: mod_base = OTHER; note = propyne dU
  modified_base 21: mod_base = m5c
tacattcctaacgtggatgtcag

SEQ ID NO: 118 - DNA, 25 bp, synthetic construct
  misc_feature 1..25: note = Synthetic Oligonucleotide
  source 1..25: mol_type = other DNA
  modified_base 2: mod_base = m5c
  modified_base 15..16: mod_base = m5c
  modified_base 20: mod_base = OTHER; note = propyne dU
  modified_base 24: mod_base = m5c
gctattatgtgtgtccatttatacg

SEQ ID NO: 119 - DNA, 25 bp, synthetic construct
  misc_feature 1..25: note = Synthetic Oligonucleotide
  source 1..25: mol_type = other DNA
  modified_base 3: mod_base = m5c
  modified_base 4: mod_base = OTHER; note = propyne dU
  modified_base 6: mod_base = m5c
  modified_base 9: mod_base = OTHER; note = propyne dU
  modified_base 13..14: mod_base = m5c
  modified_base 17: mod_base = OTHER; note = propyne dU
  modified_base 19: mod_base = m5c
  modified_base 20: mod_base = OTHER; note = propyne dU
  modified_base 22: mod_base = m5c
ttctacttttggccagttctacaat

SEQ ID NO: 120 - DNA, 26 bp, synthetic construct
  misc_feature 1..26: note = Synthetic Oligonucleotide
  source 1..26: mol_type = other DNA
  modified_base 2: mod_base = m5c
  modified_base 4: mod_base = OTHER; note = propyne dU
  modified_base 5..6: mod_base = m5c
  modified_base 8: mod_base = OTHER; note = propyne dU
  modified_base 10: mod_base = OTHER; note = propyne dU
  modified_base 13: mod_base = OTHER; note = propyne dU
  modified_base 15: mod_base = OTHER; note = propyne dU
  modified_base 20: mod_base = m5c
  modified_base 24: mod_base = OTHER; note = propyne dU
acatccatgtgatatggaacaaattg

SEQ ID NO: 121 - DNA, 25 bp, synthetic construct
  misc_feature 1..21: note = Synthetic Oligonucleotide
  source 1..25: mol_type = other DNA
cctctagcggttgtgggtgatacgg

SEQ ID NO: 122 - DNA, 24 bp, synthetic construct
  misc_feature 1..24: note = Synthetic Oligonucleotide
  source 1..24: mol_type = other DNA
  modified_base 13: mod_base = m5c
  modified_base 18: mod_base = m5c
gggtgtgataagcattgcattaac

SEQ ID NO: 123 - skipped

SEQ ID NO: 124 - DNA, 26 bp, synthetic construct
  misc_feature 1..26: note = Synthetic Oligonucleotide
  source 1..26: mol_type = other DNA
  modified_base 2..3: mod_base = m5c
  modified_base 5: mod_base = OTHER; note = propyne dU
  modified_base 6: mod_base = m5c
  modified_base 8: mod_base = m5c
  modified_base 17: mod_base = OTHER; note = propyne dU
  modified_base 20: mod_base = m5c
  modified_base 23: mod_base = OTHER; note = propyne dU
cccatcacggtgaaatttacagtgac

SEQ ID NO: 125 - DNA, 23 bp, synthetic construct
  misc_feature 1..23: note = Synthetic Oligonucleotide
  source 1..23: mol_type = other DNA
  modified_base 5..6: mod_base = m5c
  modified_base 7: mod_base = OTHER; note = propyne dU
  modified_base 11: mod_base = m5c
  modified_base 12: mod_base = OTHER; note = propyne dU
  modified_base 15: mod_base = OTHER; note = propyne dU
  modified_base 19: mod_base = OTHER; note = propyne dU
  modified_base 22: mod_base = m5c
gaagcctggactgattatttaca

SEQ ID NO: 126 - DNA, 27 bp, synthetic construct
  misc_feature 1..27: note = Synthetic Oligonucleotide
  source 1..27: mol_type = other DNA
  modified_base 7: mod_base = m5c
  modified_base 9: mod_base = m5c
  modified_base 20: mod_base = m5c
  modified_base 23: mod_base = i
  modified_base 24: mod_base = m5c
cgaaatcacgagattgatgccanccat

SEQ ID NO: 127 - DNA, 21 bp, synthetic construct
  misc_feature 1..21: note = Synthetic Oligonucleotide
  source 1..21: mol_type = other DNA
  modified_base 2: mod_base = m5c
  modified_base 4: mod_base = m5c
  modified_base 6..7: mod_base = m5c
  modified_base 12: mod_base = m5c
ccacgccaatacgatgattgt

SEQ ID NO: 128 - DNA, 24 bp, synthetic construct
  misc_feature 1..24: note = Synthetic Oligonucleotide
  source 1..24: mol_type = other DNA
  modified_base 2: mod_base = m5c
  modified_base 5: mod_base = m5c
  modified_base 10: mod_base = OTHER; note = propyne dU
  modified_base 15: mod_base = OTHER; note = propyne dU
  modified_base 17: mod_base = OTHER; note = propyne dU
  modified_base 19: mod_base = OTHER; note = propyne dU
  modified_base 22: mod_base = m5c
  modified_base 23: mod_base = OTHER; note = propyne dU
ccaacaaagttggatgtatggctt

SEQ ID NO: 129 - DNA, 25 bp, synthetic construct
  misc_feature 1..25: note = Synthetic Oligonucleotide
  source 1..25: mol_type = other DNA
  modified_base 8: mod_base = m5c
  modified_base 20: mod_base = m5c
ggcaaatcaggaagtaaaacactgg

SEQ ID NO: 130 - DNA, 28 bp, synthetic construct
  misc_feature 1..28: note = Synthetic Oligonucleotide
  source 1..28: mol_type = other DNA
  modified_base 10: mod_base = OTHER; note = propyne dU
  modified_base 15..16: mod_base = OTHER; note = propyne dU
  modified_base 26: mod_base = OTHER; note = propyne dU
  modified_base 9: mod_base = m5c
  modified_base 13: mod_base = m5c
caagagaactttctttgagggagaattt

SEQ ID NO: 131 - DNA, 27 bp, synthetic construct
  misc_feature 1..27: note = Synthetic Oligonucleotide
  source 1..27: mol_type = other DNA
  modified_base 5: mod_base = m5c
  modified_base 8: mod_base = OTHER; note = propyne dU
  modified_base 10: mod_base = m5c
  modified_base 16: mod_base = OTHER; note = propyne dU
  modified_base 18: mod_base = m5c
  modified_base 24: mod_base = m5c
agggcagttcagtgatgcattatcgac

SEQ ID NO: 132 - DNA, 21 bp, synthetic construct
  misc_feature 1..21: note = Synthetic Oligonucleotide
  source 1..21: mol_type = other DNA
  modified_base 2: mod_base = m5c
  modified_base 3: mod_base = OTHER; note = propyne dU
  modified_base 5: mod_base = m5c
  modified_base 8: mod_base = OTHER; note = propyne dU
  modified_base 12..13: mod_base = m5c
  modified_base 16: mod_base = OTHER; note = propyne dU
  modified_base 18: mod_base = m5c
  modified_base 19: mod_base = OTHER; note = propyne dU
  modified_base 21: mod_base = m5c
tctacttttggccagttctac

SEQ ID NO: 133 - DNA, 18 bp, synthetic construct
  misc_feature 1..17: note = Synthetic Oligonucleotide
  source 1..18: mol_type = other DNA
  modified_base 6: mod_base = i
atgatngtgcgcttgctg

SEQ ID NO: 134 - DNA, 27 bp, synthetic construct
  misc_feature 1..26: note = Synthetic Oligonucleotide
  source 1..27: mol_type = other DNA
  modified_base 9: mod_base = i
actaggtanattaattccacgccaacc

SEQ ID NO: 135 - DNA, 27 bp, synthetic construct
  misc_feature 1..26: note = Synthetic Oligonucleotide
  source 1..27: mol_type = other DNA
  modified_base 10: mod_base = i
acagggtaanaaactggccttgtttcg

SEQ ID NO: 136 - DNA, 22 bp, synthetic construct
  source 1..22: mol_type = other DNA
aaatgcggttgccacttaaacc

(19) United States
(12) Patent Application Publication
(10) Pub. No.: US 2026/0218268 A1
(43) Pub. Date: Jul. 30, 2026

(54) COMPOSITIONS AND METHODS FOR DETECTING GASTROINTESTINAL PATHOGENS

(71) Applicant: Gen-Probe Incorporated, San Diego, CA (US)

(72) Inventors: Nesreen H. BARAKAT, El Cajon, CA (US); Sree D. PANUGANTI, Escondido, CA (US); Tamara J. JOHNSON, San Diego, CA (US)

(21) Appl. No.: 19/146,189

(22) PCT Filed: Dec. 15, 2023
(86) PCT No.: PCT/US2023/084251

§ 371 Date: Jul. 7, 2025

Related U.S. Application Data
(60) Provisional application No. 63/476,006, filed on Dec. 19, 2022.

(51) Int. Cl. C12Q 1/04; C12Q 1/6813; C12Q 1/6844; C12Q 1/689; C12R 1/19; C12R 1/63

(52) U.S. Cl. CPC C12Q 1/04; C12Q 1/6813; C12Q 1/6844; C12Q 1/689; C12Q 2600/16; C12R 2001/19; C12R 2001/63

(57) ABSTRACT

Disclosed are nucleic acid oligomers, including amplification oligomers and detection probes, for detection of Yersinia enterocolitica, Vibrio spp., Shiga Toxin Expressing E. coli (STEC) O157, and Plesiomonas shigelloides target nucleic acid. Also disclosed are methods of specific nucleic acid amplification and detection using the disclosed oligomers, as well as corresponding formulations, reaction mixtures, and kits. Methods of synthesizing the nucleic acid oligomers are also disclosed.

### CROSS-REFERENCE TO RELATED APPLICATIONS

This application claims the benefit of U.S. Provisional Application No. 63/476,006, filed Dec. 19, 2022, which is incorporated by reference herein in its entirety.

### REFERENCE TO SEQUENCE LISTING

The instant application contains a Sequence Listing which has been submitted electronically in XML format and is hereby incorporated by reference in its entirety. Said XML Copy, created on Dec. 7, 2023, is named “4340_P23WO_Seq_Listing” and is 231,342 bytes in size.

### BACKGROUND

Bacterial gastroenteritis is inflammation of the stomach and intestines that results in acute diarrhea (3 or more episodes per day) lasting less than 14 days and may also include symptoms such as nausea, vomiting, and abdominal cramping. See Thiehnan and Guerrant, The New England Journal of Medicine, 350:38-47, 2004. In the United States, it is estimated that there are more than 200 million cases of diarrheal illness per year, resulting in 73 million physician consultations, 1.8 million hospitalizations, and up to 6,000 deaths. See Guerrant et al., Clinical Infectious Diseases, 32:331-350, 2001. Some common causes of bacterial gastroenteritis include Yersinia enterocolitica, Vibrio spp. (e.g., V. parahaemolyticus, V. vulnificus, and V. cholerae), Shiga Toxin Expressing E. coli (STEC) O157, and Plesiomonas shigelloides. The populations most at risk due to bacterial gastroenteritis infection are children (≤5), the elderly, and the immunocompromised. Infection, however, can occur in all age groups. The mode of infection is via the fecal-oral route typically from ingesting contaminated food or water or as a result of poor hygiene (hand-washing).

Yersiniosis is an infection caused most often by eating raw or undercooked pork contaminated with Yersinia enterocolitica bacteria. Pigs are the major animal reservoir for the few strains of Y. enterocolitica that cause human illness, but rodents, rabbits, sheep, cattle, horses, dogs, and cats also can carry strains that cause human illness. Yersinia enterocolitica is a gram-negative, Bacillus-shaped bacterium, belonging to the family Yersiniaceae. The Centers for Disease Control and Prevention (CDC) estimates 117.000 illnesses per year in the U.S., occurring most often in young children.

Vibrio is a genus of Gram-negative bacteria of which several species are known to cause foodborne infection associated with eating raw seafood, particularly oysters. Vibrio bacteria naturally live-in coastal waters and are present in higher concentrations between May and October when 80% of infections occur. CDC estimates that Vibrio species causes 80,000 illnesses each year in the United States. About 52,000 of these illnesses are estimated to be the result of eating contaminated food causing gastroenteritis. The most common species causing human gastroenteritis are Vibrio parahaemolyticus, Vibrio vulnificus, and Vibrio cholerae with Vibrio parahaemolyticus being the most reported species.

Shigatoxigenic E. coli (STEC) are gram-negative bacteria in the Enterobacteriaceae family. STEC are also referred to as Verotoxigenic E. coli (VTEC) or Enterohemorrhagic E. coli (EHEC). STEC are common sources of shiga toxins and are associated with, e.g., hemolytic uremic syndrome, which can be life threatening. According to CDC, 265,000 STEC infections are estimated each year in the US. STEC includes several serotypes: O157, O26, O111, O113, O2, O91, O103, O104, O45, O121, O145, and O118, O157 alone causes 36% of reported STEC infections.

Plesiomonas shigelloides is a facultatively anaerobic grain-negative bacillus found in soil fresh or estuarine (brackish) waters. It has emerged as a cause of enteric disease in humans, especially following the consumption of raw seafood and untreated water. Data suggest the incidence of Plesiomonas associated enteritis in the US is lower than 1%. Internationally, plesiomonads recovered as enteric pathogens in diarrheal stools range from 2% to 10%. See Janda, “Plesiomonas shigelloides Revisited.” Clinical Microbiology Reviews, 29(2):49-374, 2016, doi: 10.1128/CMR.00103-15.

There is a need to efficiently and sensitively detect the presence of Yersinia enterocolitica. Vibrio spp., STEC O157, and Plesiomonas shigelloides in samples, including biological specimens to provide diagnostic and prognostic information to physicians treating patients suffering from, or suspected of suffering from, bacterial gastroenteritis or related disorders.

### SUMMARY

In some aspects, the present invention provides a composition or kit for determining the presence or absence of at least one enteric pathogen in a sample, wherein the at least one enteric pathogen is selected from the group consisting of Yersinia enterocolitica, Vibrio spp., Shiga Toxin Expressing E. coli (STEC) O157, and Plesiomonas shigelloides. In one such aspect, the composition or kit generally includes a set of oligonucleotides comprising at least one of (a) a Yersinia-specific amplification oligomer set capable of amplifying a target region of a Yersinia enterocolitica target nucleic acid, (b) a Vibrio-specific amplification oligomer set capable of amplifying a target region of a Vibrio spp. target nucleic acid, wherein the Vibrio spp. is one or more of V. parahaemolyticus, V. vulimficus, and V. cholerae, (c) a STEC-specific amplification oligomer set capable of amplifying a target region of a STEC O157 target nucleic acid, and (d) a Plesiomonas-specific amplification oligomer set capable of amplifying a target region of a Plesiomonas shigelloides target nucleic acid. In another, non-mutually exclusive aspect, the composition or kit generally includes at least one detection probe oligomer capable of hybridizing to a target region of a Yersinia enterocolitica, Vibrio spp., STEC O157, or Plesiomonas shigelloides target nucleic acid or to an amplicon of said target region.

In another aspect, the present invention provides an oligonucleotide for determining the presence or absence of an enteric pathogen selected from the group consisting of Yersinia enterocolitica. Vibrio spp., Shiga Toxin Expressing E. coli (STEC) O157, and Plesiomonas shigelloides, wherein said oligonucleotide comprises a nucleotide sequence selected from the group consisting of SEQ ID NOs:1-13, 15-22, 24-35, 37, 40-68, and 70-107, including from 0 to 16 nucleotide analogs.

In other aspects, the present invention provides a reaction mixture for determining the presence or absence of at least one enteric pathogen in a sample, wherein the at least one enteric pathogen is selected from the group consisting of Yersinia enterocolitica, Vibrio spp., Shiga Toxin Expressing E. coli (STEC) O157, and Plesiomonas shigelloides. In one such aspect, the composition or kit generally includes a set of oligonucleotides comprising at least one of (a) a Yersinia-specific amplification oligomer set capable of amplifying a target region of a Yersinia enterocolitica target nucleic acid, (b) a Vibrio-specific amplification oligomer set capable of amplifying a target region of a Vibrio spp. target nucleic acid, wherein the Vibrio spp. is one or more of V. parahaemolyticus, V. vulnificus, and V. cholerae, (c) a STEC-specific amplification oligomer set capable of amplifying a target region of a STEC O157 target nucleic acid, and (d) a Plesiomonas-specific amplification oligomer set capable of amplifying a target region of a Plesiomonas shigelloides target nucleic acid. In another, non-mutually exclusive aspect, the reaction mixture generally includes at least one detection probe oligomer capable of hybridizing to a target region of a Yersinia enterocolitica, Vibrio spp., STEC O157, or Plesiomonas shigelloides target nucleic acid or to an amplicon of said target region. In yet another non-mutually exclusive aspect, the reaction mixture comprises an oligonucleotide comprising a nucleotide sequence selected from the group consisting of SEQ ID NOs:1-13, 15-22, 24-35, 37, 40-68, and 70-107, including from 0 to 16 nucleotide analogs.

In another aspect, the present invention provides a method for determining the presence or absence of at least one enteric pathogen in a sample, wherein the at least one enteric pathogen is selected from the group consisting of Yersinia enterocolitica, Vibrio spp., Shiga Toxin Expressing E. coli (STEC) O157, and Plesiomonas shigelloides. The method generally includes performing an in vitro nucleic acid amplification reaction, utilizing an oligomer combination capable of amplifying a target region of Yersinia enterocolitica, Vibrio spp., STEC O157, and/or Plesiomonas shigelloides target nucleic acid, to generate one or more amplification products corresponding to the Yersinia enterocolitica, Vibrio spp., STEC O157, and/or Plesiomonas shigelloides target region, and detecting the presence or absence of the one or more amplification products.

In another aspect, the present invention provides a method for synthesizing an oligonucleotide, wherein the oligonucleotide comprises a nucleotide sequence selected from the group consisting of SEQ ID NOs:1-13, 15-22, 24-35, 37, 40-68, 70-107, and 121, including from 0 to 16 nucleotide analogs. In a related aspect, the present invention provides a method for synthesizing a pair of oligonucleotides, comprising synthesizing a first oligonucleotide and synthesizing a second oligonucleotide, wherein the first oligonucleotide and the second oligonucleotide target sequences flanking a target region of a Yersinia enterocolitica, Vibrio spp., STEC O157, or Plesiomonas shigelloides target nucleic acid, and wherein the first oligonucleotide and the second oligonucleotide each comprise a nucleotide sequence selected from the group consisting of SEQ ID NOs:1-6, 10, 11, 13, 16, 17, 19-22, 24, 26, 28, 30, 32, 37, 41-47, 49, 51-58, 60, 62, 64-66, 68, 70, 71, 75-79, 81, 84-87, 90, 92-95, 97, 98, 100-102, and 105-107, including from 0 to 16 nucleotide analogs.

Representative embodiments of these aspects are further set forth below.

### Embodiments

Embodiment 1. A combination or kit for determining the presence or absence of at least one enteric pathogen in a sample, wherein the at least one enteric pathogen is selected from the group consisting of Yersinia enterocolitica, Vibrio spp., Shiga Toxin Expressing E. coli (STEC) O157, and Plesiomonas shigelloides, said composition or kit comprising a set of oligonucleotides comprising at least one of (a)-(d):
(a) a Yersinia-specific amplification oligomer set capable of amplifying a target region of a Yersinia enterocolitica target nucleic acid, wherein the Yersinia-specific amplification oligomer set comprises first and second Yersinia-specific amplification oligomers respectively comprising target-hybridizing sequences substantially corresponding to the nucleotide sequences of (i) SEQ ID NO:26 and SEQ ID NO:46, (ii) SEQ ID NO:97 and SEQ ID NO:4, (iii) SEQ ID NO:102 and SEQ ID NO:24, (iv) SEQ ID NO:6 and SEQ ID NO:24, (v) SEQ ID NO:13 and SEQ ID NO:22, (vi) SEQ ID NO:77 and SEQ ID NO:22. (vii) SEQ ID NO:77 and SEQ ID NO:41, (viii) SEQ ID NO:28 and SEQ ID NO:30, (ix) SEQ ID NO:60 and SEQ ID NO:45. (x) SEQ ID NO:65 and SEQ ID NO:54, or (xi) SEQ ID NO:10 and SEQ ID NO:4;
(b) a Vibrio-specific amplification oligomer set capable of amplifying a target region of a Vibrio spp. target nucleic acid, wherein the Vibrio spp. is one or more of V parahaemolyticus, V. vulnificus, and V. cholerae, and wherein the Vibrio-specific amplification oligomer set comprises
   (b-1) first and second V. parahaemolyticus-specific amplification oligomers respectively comprising target-hybridizing sequences substantially corresponding to the nucleotide sequences of (i) SEQ ID NO:51 and SEQ ID NO:76, (ii) SEQ ID NO:58 and SEQ ID NO:21, (iii) SEQ ID NO:58 and SEQ ID NO:86, (iv) SEQ ID NO:53 and SEQ ID NO:75, (v) SEQ ID NO:58 and SEQ ID NO:20, (vi) SEQ ID NO:52 and SEQ ID NO:75, (vii) SEQ ID NO:51 and SEQ ID NO:75, (viii) SEQ ID NO:52 and SEQ ID NO:76, or (ix) SEQ ID NO:53 and SEQ ID NO:76; and/or
   (b-2) first and second V. vulnificus-specific amplification oligomers respectively comprising target-hybridizing sequences substantially corresponding to the nucleotide sequences of (i) SEQ ID NO:66 and SEQ ID NO:62, (ii) SEQ ID NO:58 and SEQ ID NO:21, (iii) SEQ ID NO:42 and SEQ ID NO:37. (iv) SEQ ID NO:55 and SEQ ID NO:37. (v) SEQ ID NO:43 and SEQ ID NO:78, (vi) SEQ ID NO:58 and SEQ ID NO:20, (vii) SEQ ID NO:42 and SEQ ID NO:84, (viii) SEQ ID NO:42 and SEQ ID NO:47, (ix) SEQ ID NO:55 and SEQ ID NO:84. (x) SEQ ID NO:55 and SEQ ID NO:47, or (xi) SEQ ID NO:55 and SEQ ID NO:5; and/or
   (b-3) first and second V. cholerae-specific amplification oligomers respectively comprising target-hybridizing sequences substantially corresponding to the nucleotide sequences of (i) SEQ ID NO:56 and SEQ ID NO:11. (ii) SEQ ID NO:32 and SEQ ID NO:3. (iii) SEQ ID NO:57 and SEQ ID NO:64, (iv) SEQ ID NO:94 and SEQ ID NO:19. (v) SEQ ID NO:32 and SEQ ID NO:105, (vi) SEQ ID NO:92 and SEQ ID NO:3; (vii) SEQ ID NO:92 and SEQ ID NO:105, (viii) SEQ ID NO:85 and SEQ ID NO:3, or (ix) SEQ ID NO:85 and SEQ ID NO:105;
(c) a STEC-specific amplification oligomer set capable of amplifying a target region of a STEC O157 target nucleic acid, wherein the STEC-specific amplification oligomer set comprises first and second STEC-specific amplification oligomers respectively comprising target-hybridizing sequences substantially corresponding to the nucleotide sequences of (i) SEQ ID NO:70 and SEQ ID NO: 16, (ii) SEQ ID NO:93 and SEQ ID NO:101, (iii) SEQ ID NO:71 and SEQ ID NO:44, (iv) SEQ ID NO:90 and SEQ ID NO:100, (v) SEQ ID NO:71 and SEQ ID NO:16, (vi) SEQ ID NO:70 and SEQ ID NO:44. (vii) SEQ ID NO:70 and SEQ ID NO:107, (viii) SEQ ID NO:70 and SEQ ID NO:87, (ix) SEQ ID NO:71 and SEQ ID NO:107, or (x) SEQ ID NO:71 and SEQ ID NO:87; and
(d) a Plesiomonas-specific amplification oligomer set capable of amplifying a target region of a Plesiomonas shigelloides target nucleic acid, wherein the Plesiomonas-specific amplification oligomer set comprises first and second Plesiomonas-specific amplification oligomers respectively comprising target-hybridizing sequences substantially corresponding to the nucleotide sequences of (i) SEQ ID NO:2 and SEQ ID NO:49, (ii) SEQ ID NO:95 and SEQ ID NO:1, (iii) SEQ ID NO:79 and SEQ ID NO:81. (iv) SEQ ID NO:17 and SEQ ID NO:1, (v) SEQ ID NO:95 and SEQ ID NO:106, (vi) SEQ ID NO:17 and SEQ ID NO:106,
(vii) SEQ ID NO:79 and SEQ ID NO:68, (viii) SEQ ID NO:17 and SEQ ID NO:98, or (ix) SEQ ID NO:95 and SEQ ID NO:98.

Embodiment 2. The composition or kit of Embodiment 1, wherein the set of oligonucleotides comprises the Yersinia-specific amplification oligomer set.

Embodiment 3. The composition or kit of Embodiment 2, wherein the set of oligonucleotides further comprises a Yersinia-specific detection probe comprising a target-hybridizing sequence substantially corresponding to the nucleotide sequence of
SEQ ID NO:40 if the Yersinia-specific amplification oligomer set comprises first and second Yersinia-specific amplification oligomers respectively comprising target-hybridizing sequences substantially corresponding to the nucleotide sequences of SEQ ID NO:26 and SEQ ID NO:46;
SEQ ID NO:50 if the Yersinia-specific amplification oligomer set comprises first and second Yersinia-specific amplification oligomers respectively comprising target-hybridizing sequences substantially corresponding to the nucleotide sequences of (i) SEQ ID NO:97 and SEQ ID NO:4 or (ii) SEQ ID NO:10 and SEQ ID NO:4;
SEQ ID NO:63 or SEQ ID NO:83 if the Yersinia-specific amplification oligomer set comprises first and second Yersinia-specific amplification oligomers respectively comprising target-hybridizing sequences substantially corresponding to the nucleotide sequences of (i) SEQ ID NO:102 and SEQ ID NO:24 or (ii) SEQ ID NO:6 and SEQ ID NO:24;
SEQ ID NO:29 if the Yersinia-specific amplification oligomer set comprises first and second Yersinia-specific amplification oligomers respectively comprising target-hybridizing sequences substantially corresponding to the nucleotide sequences of (i) SEQ ID NO:13 and SEQ ID NO:22 or (ii) SEQ ID NO:77 and SEQ ID NO:22;
SEQ ID NO:34 if the Yersinia-specific amplification oligomer set comprises first and second Yersinia-specific amplification oligomers respectively comprising target-hybridizing sequences substantially corresponding to the nucleotide sequences of SEQ ID NO:77 and SEQ ID NO:41;
SEQ ID NO:27 if the Yersinia-specific amplification oligomer set comprises first and second Yersinia-specific amplification oligomers respectively comprising target-hybridizing sequences substantially corresponding to the nucleotide sequences of SEQ ID NO:28 and SEQ ID NO:30;
SEQ ID NO:25 if the Yersinia-specific amplification oligomer set comprises first and second Yersinia-specific amplification oligomers respectively comprising target-hybridizing sequences substantially corresponding to the nucleotide sequences of SEQ ID NO:60 and SEQ ID NO:45: or
SEQ ID NO:18 if the Yersinia-specific amplification oligomer set comprises first and second Yersinia-specific amplification oligomers respectively comprising target-hybridizing sequences substantially corresponding to the nucleotide sequences of SEQ ID NO:65 and SEQ ID NO:54.

Embodiment 4. The composition or kit of any one of Embodiments 1 to 3, wherein the set of oligonucleotides comprises the Vibrio-specific amplification oligomer set.

Embodiment 5. The composition or kit of Embodiment 4, wherein the Vibrio-specific amplification oligomer set comprises the first and second V. parahaemolyticus-specific amplification oligomers.

Embodiment 6. The composition or kit of Embodiment 5, wherein the set of oligonucleotides further comprises a V. parahaemolyticus-specific detection probe comprising a target-hybridizing sequence substantially corresponding to the nucleotide sequence of
SEQ ID NO:33, SEQ ID NO:89, SEQ ID NO:91, or SEQ ID NO:121 if the V parahaemolyticus-specific amplification oligomer set comprises first and second V. parahaemolyticus-specific amplification oligomers respectively comprising target-hybridizing sequences substantially corresponding to the nucleotide sequences of (i) SEQ ID NO:51 and SEQ ID NO:76, (ii) SEQ ID NO:53 and SEQ ID NO:75, (iii) SEQ ID NO:52 and SEQ ID NO:75, (iv) SEQ ID NO:51 and SEQ ID NO:75, (v) SEQ ID NO:52 and SEQ ID NO:76, or (vi) SEQ ID NO:53 and SEQ ID NO:76; or
SEQ ID NO:15 if the V. parahaemolyticus-specific amplification oligomer set comprises first and second V. parahaemolyticus-specific amplification oligomers respectively comprising target-hybridizing sequences substantially corresponding to the nucleotide sequences of (i) SEQ ID NO:58 and SEQ ID NO:21, (ii) SEQ ID NO:58 and SEQ ID NO:86, or (iii) SEQ ID NO:58 and SEQ ID NO:20.

Embodiment 7. The composition or kit of any one of Embodiments 4 to 6, wherein the Vibrio-specific amplification oligomer set comprises the first and second V. vulnificus-specific amplification oligomers.

Embodiment 8. The composition or kit of Embodiment 7, wherein the set of oligonucleotides further comprises a V. vulnificus-specific detection probe comprising a target-hybridizing sequence substantially corresponding to the nucleotide sequence of
SEQ ID NO:9 if the V. vulnificus-specific amplification oligomer set comprises first and second V. vulnificus-specific amplification oligomers respectively comprising target-hybridizing sequences substantially corresponding to the nucleotide sequences of (i) SEQ ID NO:66 and SEQ ID NO:62, (ii) SEQ ID NO:42 and SEQ ID NO:37, (iii) SEQ ID NO:55 and SEQ ID NO:37, (iv) SEQ ID NO:42 and SEQ ID NO:84, (v) SEQ ID NO:42 and SEQ ID NO:47, (vi) SEQ ID NO:55 and SEQ ID NO:84, (vii) SEQ ID NO:55 and SEQ ID NO:47, or (viii) SEQ ID NO:55 and SEQ ID NO:5; or
SEQ ID NO:31 if the V. vulnificus-specific amplification oligomer set comprises first and second V. vulnificus-specific amplification oligomers respectively comprising target-hybridizing sequences substantially corresponding to the nucleotide sequences of (i) SEQ ID NO:58 and SEQ ID NO:21, (ii) SEQ ID NO:58 and SEQ ID NO:20, (iii) or (ii) SEQ ID NO:43 and SEQ ID NO:78.

Embodiment 9. The composition or kit of any one of Embodiments 4 to 8, wherein the Vibrio-specific amplification oligomer set comprises the first and second V. cholerae-specific amplification oligomers.

The composition or kit of Embodiment 9, wherein the set of oligonucleotides further comprises a V. cholerae-specific detection probe comprising a target-hybridizing sequence substantially corresponding to the nucleotide sequence of
SEQ ID NO:12 if the V. cholerae-specific amplification oligomer set comprises first and second V. cholerae-specific amplification oligomers respectively comprising target-hybridizing sequences substantially corresponding to the nucleotide sequences of SEQ ID NO:56 and SEQ ID NO:11;
SEQ ID NO:35 or SEQ ID NO:88 if the V. cholerae-specific amplification oligomer set comprises first and second V. cholerae-specific amplification oligomers respectively comprising target-hybridizing sequences substantially corresponding to the nucleotide sequences of (i) SEQ ID NO:32 and SEQ ID NO:3, (ii) SEQ ID NO:57 and SEQ ID NO:64, (iii) SEQ ID NO:32 and SEQ ID NO:105, (iv) SEQ ID NO:92 and SEQ ID NO:3; (v) SEQ ID NO:92 and SEQ ID NO:105, (vi) SEQ ID NO:85 and SEQ ID NO:3, or (vii) SEQ ID NO:85 and SEQ ID NO:105; or
SEQ ID NO:67 or SEQ ID NO:82 if the V. cholerae-specific amplification oligomer set comprises first and second V. cholerae-specific amplification oligomers respectively comprising target-hybridizing sequences substantially corresponding to the nucleotide sequences of SEQ ID NO:94 and SEQ ID NO:19.

Embodiment 11. The composition or kit of any one of Embodiments 1 to 10, wherein the set of oligonucleotides comprises the STEC-specific amplification oligomer set.

Embodiment 12. The composition or kit of Embodiment 11, wherein the set of oligonucleotides further comprises a STEC-specific detection probe comprising a target-hybridizing sequence substantially corresponding to the nucleotide sequence of
SEQ ID NO:73, SEQ ID NO:74, SEQ ID NO:7, or SEQ ID NO:59 if the STEC-specific amplification oligomer set comprises first and second STEC-specific amplification oligomers respectively comprising target-hybridizing sequences substantially corresponding to the nucleotide sequences of (i) SEQ ID NO:70 and SEQ ID NO:16, (ii) SEQ ID NO:71 and SEQ ID NO:44, (iii) SEQ ID NO:71 and SEQ ID NO:16, (iv) SEQ ID NO:70 and SEQ ID NO:44. (v) SEQ ID NO:70 and SEQ ID NO:107. (vi) SEQ ID NO:70 and SEQ ID NO:87, (vii) SEQ ID NO:71 and SEQ ID NO:107, or (viii) SEQ ID NO:71 and SEQ ID NO:87;
SEQ ID NO:96 or SEQ ID NO:103 if the STEC-specific amplification oligomer set comprises first and second STEC-specific amplification oligomers respectively comprising target-hybridizing sequences substantially corresponding to the nucleotide sequences of SEQ ID NO:90 and SEQ ID NO:100; or
SEQ ID NO:99 or SEQ ID NO:104 if the STEC-specific amplification oligomer set comprises first and second STEC-specific amplification oligomers respectively comprising target-hybridizing sequences substantially corresponding to the nucleotide sequences of SEQ ID NO:93 and SEQ ID NO:101.

Embodiment 13. The composition or kit of any one of Embodiments 1 to 12, wherein the set of oligonucleotides comprises the Plesiomonas-specific amplification oligomer set.

Embodiment 14. The composition or kit of Embodiment 13, wherein the set of oligonucleotides further comprises a Plesiomonas-specific detection probe comprising a target-hybridizing sequence substantially corresponding to the nucleotide sequence of
SEQ ID NO:48 if the Plesiomonas-specific amplification oligomer set comprises first and second Plesiomonas-specific amplification oligomers respectively comprising target-hybridizing sequences substantially corresponding to the nucleotide sequences of (i) SEQ ID NO:2 and SEQ ID NO:49 or (ii) SEQ ID NO:17 and SEQ ID NO:98;
SEQ ID NO:80, SEQ ID NO:72, or SEQ ID NO:61 if the Plesiomonas-specific amplification oligomer set comprises first and second Plesiomonas-specific amplification oligomers respectively comprising target-hybridizing sequences substantially corresponding to the nucleotide sequences of (i) SEQ ID NO:95 and SEQ ID NO:1, (ii) SEQ ID NO:17 and SEQ ID NO:1, (iii) SEQ ID NO:95 and SEQ ID NO:106. (iv) SEQ ID NO:17 and SEQ ID NO:106, (v) SEQ ID NO: 17 and SEQ ID NO:98, or (vi) SEQ ID NO:95 and SEQ ID NO:98; or
SEQ ID NO:8 if the Plesiomonas-specific amplification oligomer set comprises first and second Plesiomonas-specific amplification oligomers respectively comprising target-hybridizing sequences substantially corresponding to the nucleotide sequences of (i) SEQ ID NO:79 and SEQ ID NO:81 or (ii) SEQ ID NO:79 and SEQ ID NO:68.

Embodiment 15. The composition or kit of any one of Embodiments 1 to 14, wherein the set of oligonucleotides comprises at least two of the Yersinia-specific amplification oligomer set, the Vibrio-specific amplification oligomer set, the STEC-specific amplification oligomer set, and the Plesiomonas-specific amplification oligomer set.

Embodiment 16. The composition or kit of any one of Embodiments 1 to 14, wherein the set of oligonucleotides comprises at least three of the Yersinia-specific amplification oligomer set, the Vibrio-specific amplification oligomer set, the STEC-specific amplification oligomer set, and the Plesiomonas-specific amplification oligomer set.

Embodiment 17. The composition or kit of Embodiment 1, wherein the set of oligonucleotides comprises the Yersinia-specific amplification oligomer set, the Vibrio-specific amplification oligomer set, the STEC-specific amplification oligomer set, and the Plesiomonas-specific amplification oligomer set.

Embodiment 18. The composition or kit of Embodiment 17, wherein the Yersinia-specific amplification oligomer set comprises first and second Yersinia-specific amplification oligomers respectively comprising target-hybridizing sequences substantially corresponding to the nucleotide sequences of SEQ ID NO:26 and SEQ ID NO:46.

Embodiment 19. The composition or kit of Embodiment 18, wherein the first Yersinia-specific amplification oligomer comprises a target-hybridizing sequence as shown in SEQ ID NO:26, including from 0 to 16 nucleotide analogs; and/or the second Yersinia-specific amplification oligomer comprises a target-hybridizing sequence as shown in SEQ ID NO:46, including from 0 to 16 nucleotide analogs.

Embodiment 20. The composition or kit of Embodiment 18 or 19, wherein the set of oligonucleotides further comprises a Yersinia-specific detection probe comprising a target-hybridizing sequence as shown in SEQ ID NO:40, including from 0 to 16 nucleotide analogs.

Embodiment 21. The composition or kit of any one of Embodiments 17 to 20, wherein the Vibrio-specific amplification oligomer set comprises first and second V. parahaemolyticus-specific amplification oligomers respectively comprising target-hybridizing sequences substantially corresponding to the nucleotide sequences of SEQ ID NO:51 and SEQ ID NO:76.

Embodiment 22. The composition or kit of Embodiment 21, wherein the first V. parahaemolyticus-specific amplification oligomer comprises a target-hybridizing sequence as shown in SEQ ID NO:51, including from 0 to 16 nucleotide analogs; and/or the second V. parahaemolyticus-specific amplification oligomer comprises a target-hybridizing sequence as shown in SEQ ID NO:76, including from 0 to 16 nucleotide analogs.

Embodiment 23. The composition or kit of Embodiment 21 or 22, wherein the set of oligonucleotides further comprises a V. parahaemolyticus-specific detection probe comprising a target-hybridizing sequence as shown in SEQ ID NO:33, SEQ ID NO:89, SEQ ID NO:91, or SEQ ID NO:121 including from 0 to 16 nucleotide analogs.

Embodiment 24. The composition or kit of any one of Embodiments 17 to 23, wherein the Vibrio-specific amplification oligomer set comprises first and second V. vulnificus-specific amplification oligomers respectively comprising target-hybridizing sequences substantially corresponding to the nucleotide sequences of SEQ ID NO:66 and SEQ ID NO:62.

Embodiment 25. The composition or kit of Embodiment 24, wherein the first V. vulnificus-specific amplification oligomer comprises a target-hybridizing sequence as shown in SEQ ID NO:66, including from 0 to 16 nucleotide analogs; and/or the second V. vulnificus-specific amplification oligomer comprises a target-hybridizing sequence as shown in SEQ ID NO:62, including from 0 to 16 nucleotide analogs.

Embodiment 26. The composition or kit of Embodiment 24 or 25, wherein the set of oligonucleotides further comprises a V. vulnificus-specific detection probe comprising a target-hybridizing sequence as shown in SEQ ID NO:9, including from 0 to 16 nucleotide analogs.

Embodiment 27. The composition or kit of any one of Embodiments 17 to 26, wherein the Vibrio-specific amplification oligomer set comprises first and second V. cholerae-specific amplification oligomers respectively comprising target-hybridizing sequences substantially corresponding to the nucleotide sequences of SEQ ID NO:56 and SEQ ID NO:11.

Embodiment 28. The composition or kit of Embodiment 27, wherein the first V cholerae-specific amplification oligomer comprises a target-hybridizing sequence as shown in SEQ ID NO:56, including from 0 to 16 nucleotide analogs; and/or the second V. cholerae-specific amplification oligomer comprises a target-hybridizing sequence as shown in SEQ ID NO:11, including from 0 to 16 nucleotide analogs.

Embodiment 29. The composition or kit of Embodiment 27 or 28, wherein the set of oligonucleotides further comprises a V. cholerae-specific detection probe comprising a target-hybridizing sequence as shown in SEQ ID NO:12, including from 0 to 16 nucleotide analogs.

Embodiment 30. The composition or kit of any one of Embodiments 17 to 29, wherein the STEC-specific amplification oligomer set comprises first and second STEC-specific amplification oligomers respectively comprising target-hybridizing sequences substantially corresponding to the nucleotide sequences of SEQ ID NO:70 and SEQ ID NO:16.

Embodiment 31. The composition or kit of Embodiment 30, wherein the first STEC-specific amplification oligomer comprises a target-hybridizing sequence as shown in SEQ ID NO:70, including from 0 to 16 nucleotide analogs; and/or the second STEC-specific amplification oligomer comprises a target-hybridizing sequence as shown in SEQ ID NO:16, including from 0 to 16 nucleotide analogs.

Embodiment 32. The composition or kit of Embodiment 30 or 31, wherein the set of oligonucleotides further comprises a STEC-specific detection probe comprising a target-hybridizing sequence as shown in SEQ ID NO:73, SEQ ID NO:74, SEQ ID NO:7, or SEQ ID NO:59, including from 0 to 16 nucleotide analogs.

Embodiment 33. The composition or kit of any one of Embodiments 17 to 32, wherein the Plesiomonas-specific amplification oligomer set comprises first and second Plesiomonas-specific amplification oligomers respectively comprising target-hybridizing sequences substantially corresponding to the nucleotide sequences of SEQ ID NO:2 and SEQ ID NO:49.

Embodiment 34. The composition or kit of Embodiment 33, wherein the first Plesiomonas-specific amplification oligomer comprises a target-hybridizing sequence as shown in SEQ ID NO:2, including from 0 to 16 nucleotide analogs; and/or the second Plesiomonas-specific amplification oligomer comprises a target-hybridizing sequence as shown in SEQ ID NO:49, including from 0 to 16 nucleotide analogs.

Embodiment 35. The composition or kit of Embodiment 33 or 34, wherein the set of oligonucleotides further comprises a Plesiomonas-specific detection probe comprising a target-hybridizing sequence as shown in SEQ ID NO:48, including from 0 to 16 nucleotide analogs.

Embodiment 36. The composition or kit of any one of Embodiments 20, 23, 26, 29, 32, and 35, wherein one or more of the detection probes comprises a detectable label.

Embodiment 37. The composition or kit of Embodiment 36, wherein the detectable label is a fluorescent or chemiluminescent label.

Embodiment 38. The composition or kit of Embodiment 36, wherein the detectable label is a fluorescent label and each of the one or more detection probes further comprises a non-fluorescent quencher.

Embodiment 39. The kit of any one of Embodiments 1 to 38, wherein the set of oligonucleotides are contained in formulation comprising at least one of (a) a non-linear surfactant, (b) a lyoprotectant, and (c) a chelating agent.

Embodiment 40. The kit of Embodiment 39, wherein the formulation is a lyophilized formulation.

Embodiment 41. An oligonucleotide for determining the presence or absence of an enteric pathogen, wherein said oligonucleotide comprises a nucleotide sequence selected from the group consisting of SEQ ID NOs:1-13, 15-22, 24-35, 37, 40-68, and 70-107, including from 0 to 16 nucleotide analogs.

Embodiment 42. The oligonucleotide of Embodiment 41, wherein the nucleotide sequence is selected from the group consisting of SEQ ID NOs:108-136.

Embodiment 43. The oligonucleotide of Embodiment 41 or 42, wherein the 3′ end of said oligonucleotide is attached to a solid support.

Embodiment 44. The oligonucleotide of Embodiment 43, wherein the solid support is a controlled pore glass.

Embodiment 45. A reaction mixture for determining the presence or absence of at least one enteric pathogen in a sample, said reaction mixture comprising a set of oligonucleotides as specified in any one of Embodiments 1 to 38.

Embodiment 46. A reaction mixture for determining the presence or absence of an enteric pathogen in a sample, said reaction mixture comprising the oligonucleotide of Embodiment 41 or 42.

Embodiment 47. The reaction mixture of Embodiment 45 or 46, further comprising at least one of (a) a non-linear surfactant, (b) a lyoprotectant, (c) α-cyclodextrin, and (d) a chelating agent.

Embodiment 48. A method for determining the presence or absence of at least one enteric pathogen in a sample, wherein the at least one enteric pathogen is selected from the group consisting of Yersinia enterocolitica, Vibrio spp., Shiga Toxin Expressing E. coli (STEC) O157, and Plesiomonas shigelloides, the method comprising:
(1) contacting a sample, said sample suspected of containing the at least one enteric pathogen, with an oligomer combination capable of amplifying a target region of Yersinia enterocolitica, Vibrio spp., STEC O157, and/or Plesiomonas shigelloides target nucleic acid, said oligomer combination comprising
   (a) a Yersinia-specific amplification oligomer set capable of amplifying a target region of a Yersinia enterocolitica target nucleic acid, wherein the Yersinia-specific amplification oligomer set comprises first and second Yersinia-specific amplification oligomers respectively comprising target-hybridizing sequences substantially corresponding to the nucleotide sequences of (i) SEQ ID NO:26 and SEQ ID NO:46, (ii) SEQ ID NO:97 and SEQ ID NO:4, (iii) SEQ ID NO:102 and SEQ ID NO:24, (iv) SEQ ID NO:6 and SEQ ID NO:24, (v) SEQ ID NO:13 and SEQ ID NO:22, (vi) SEQ ID NO:77 and SEQ ID NO:22, (vii) SEQ ID NO:77 and SEQ ID NO:41, (viii) SEQ ID NO:28 and SEQ ID NO:30, (ix) SEQ ID NO:60 and SEQ ID NO:45, (x) SEQ ID NO:65 and SEQ ID NO:54, or (xi) SEQ ID NO:10 and SEQ ID NO:4; and/or
   (b) a Vibrio-specific amplification oligomer set capable of amplifying a target region of a Vibrio spp. target nucleic acid, wherein the Vibrio spp. is one or more of V. parahaemolyticus. V. vulnificus, and V. cholerae, and wherein the Vibrio-specific amplification oligomer set comprises
      (b-1) first and second V. parahaemolyticus-specific amplification oligomers respectively comprising target-hybridizing sequences substantially corresponding to the nucleotide sequences of (i) SEQ ID NO:51 and SEQ ID NO:76, (ii) SEQ ID NO:58 and SEQ ID NO:21, (iii) SEQ ID NO:58 and SEQ ID NO:86, (iv) SEQ ID NO:53 and SEQ ID NO:75, (v) SEQ ID NO:58 and SEQ ID NO:20, (vi) SEQ ID NO:52 and SEQ ID NO:75, (vii) SEQ ID NO:51 and SEQ ID NO:75, (viii) SEQ ID NO:52 and SEQ ID NO:76, or (ix) SEQ ID NO:53 and SEQ ID NO:76; and/or
      (b-2) first and second V. vulnmficus-specific amplification oligomers respectively comprising target-hybridizing sequences substantially corresponding to the nucleotide sequences of (i) SEQ ID NO:66 and SEQ ID NO:62, (ii) SEQ ID NO:58 and SEQ ID NO:21, (iii) SEQ ID NO:42 and SEQ ID NO:37, (iv) SEQ ID NO:55 and SEQ ID NO:37, (v) SEQ ID NO:43 and SEQ ID NO:78, (vi) SEQ ID NO:58 and SEQ ID NO:20, (vii) SEQ ID NO:42 and SEQ ID NO:84, (viii) SEQ ID NO:42 and SEQ ID NO:47, (ix) SEQ ID NO:55 and SEQ ID NO:84, (x) SEQ ID NO:55 and SEQ ID NO:47, or (xi) SEQ ID NO:55 and SEQ ID NO:5; and/or
      (b-3) first and second V. cholerae-specific amplification oligomers respectively comprising target-hybridizing sequences substantially corresponding to the nucleotide sequences of (i) SEQ ID NO:56 and SEQ ID NO: 1, (ii) SEQ ID NO:32 and SEQ ID NO:3, (iii) SEQ ID NO:57 and SEQ ID NO:64, (iv) SEQ ID NO:94 and SEQ ID NO:19, (v) SEQ ID NO:32 and SEQ ID NO:105, (vi) SEQ ID NO:92 and SEQ ID NO:3: (vii) SEQ ID NO:92 and SEQ ID NO:105, (viii) SEQ ID NO:85 and SEQ ID NO:3, or (ix) SEQ ID NO:85 and SEQ ID NO:105; and/or
   (c) a STEC-specific amplification oligomer set capable of amplifying a target region of a STEC O157 target nucleic acid, wherein the STEC-specific amplification oligomer set comprises first and second STEC-specific amplification oligomers respectively comprising target-hybridizing sequences substantially corresponding to the nucleotide sequences of (i) SEQ ID NO:70 and SEQ ID NO:16, (ii) SEQ ID NO:93 and SEQ ID NO:101, (iii) SEQ ID NO:71 and SEQ ID NO:44, (iv) SEQ ID NO:90 and SEQ ID NO:100, (v) SEQ ID NO:71 and SEQ ID NO:16, (vi) SEQ ID NO:70 and SEQ ID NO:44, (vii) SEQ ID NO:70 and SEQ ID NO:107, (viii) SEQ ID NO:70 and SEQ ID NO:87, (ix) SEQ ID NO:71 and SEQ ID NO:107, or (x) SEQ ID NO:71 and SEQ ID NO:87; and/or
   (d) a Plesiomonas-specific amplification oligomer set capable of amplifying a target region of a Plesiomonas shigelloides target nucleic acid, wherein the Plesiomonas-specific amplification oligomer set comprises first and second Plesiomonas-specific amplification oligomers respectively comprising target-hybridizing sequences substantially corresponding to the nucleotide sequences of (i) SEQ ID NO:2 and SEQ ID NO:49, (ii) SEQ ID NO:95 and SEQ ID NO:1, (iii) SEQ ID NO:79 and SEQ ID NO:81, (iv) SEQ ID NO:17 and SEQ ID NO:1, (v) SEQ ID NO:95 and SEQ ID NO:106, (vi) SEQ ID NO:17 and SEQ ID NO:106, (vii) SEQ ID NO:79 and SEQ ID NO:68, (viii) SEQ ID NO:17 and SEQ ID NO:98, or (ix) SEQ ID NO:95 and SEQ ID NO:98;
(2) performing an in vitro nucleic acid amplification reaction, wherein any Yersinia enterocolitica, Vibrio spp., STEC O157, and/or Plesiomonas shigelloides target nucleic acid present in the sample is used as a template for generating one or more amplification products corresponding to the Yersnia enterocolitica, Vibrio spp., STEC O157, and/or Plesiomonas shigelloides target regions; and
(3) detecting the presence or absence of the one or more amplification products, thereby determining the presence or absence of the at least one enteric pathogen in the sample.

Embodiment 49. The method of Embodiment 48, wherein the sample is contacted with the Yersinia-specific amplification oligomer set and any Yersinia enterocolitica target nucleic acid present in the sample is used as a template for generating a Yersinia enterocolitica amplification product corresponding to the Yersinia enterocolitica target region.

Embodiment 50. The method of Embodiment 49, wherein the detecting step (3) comprises contacting the in vitro nucleic acid amplification reaction with a Yersinia-specific detection probe configured to specifically hybridize to the Yersinia enterocolitica amplification product.

Embodiment 51. The method of Embodiment 50, wherein the Yersinia-specific detection probe comprises a target-hybridizing sequence substantially corresponding to the nucleotide sequence of
SEQ ID NO:40 if the Yersinia-specific amplification oligomer set comprises first and second Yersinia-specific amplification oligomers respectively comprising target-hybridizing sequences substantially corresponding to the nucleotide sequences of SEQ ID NO:26 and SEQ ID NO:46;
SEQ ID NO:50 if the Yersinia-specific amplification oligomer set comprises first and second Yersinia-specific amplification oligomers respectively comprising target-hybridizing sequences substantially corresponding to the nucleotide sequences of (i) SEQ ID NO:97 and SEQ ID NO:4 or (ii) SEQ ID NO:10 and SEQ ID NO:4;
SEQ ID NO:63 or SEQ ID NO:83 if the Yersinia-specific amplification oligomer set comprises first and second Yersinia-specific amplification oligomers respectively comprising target-hybridizing sequences substantially corresponding to the nucleotide sequences of (i) SEQ ID NO:102 and SEQ ID NO:24 or (ii) SEQ ID NO:6 and SEQ ID NO:24;
SEQ ID NO:29 if the Yersinia-specific amplification oligomer set comprises first and second Yersinia-specific amplification oligomers respectively comprising target-hybridizing sequences substantially corresponding to the nucleotide sequences of (i) SEQ ID NO:13 and SEQ ID NO:22 or (ii) SEQ ID NO:77 and SEQ ID NO:22;
SEQ ID NO:34 if the Yersinia-specific amplification oligoner set comprises first and second Yersinia-specific amplification oligomers respectively comprising target-hybridizing sequences substantially corresponding to the nucleotide sequences of SEQ ID NO:77 and SEQ ID NO:41;
SEQ ID NO:27 if the Yersinia-specific amplification oligoner set comprises first and second Yersinia-specific amplification oligomers respectively comprising target-hybridizing sequences substantially corresponding to the nucleotide sequences of SEQ ID NO:28 and SEQ ID NO:30;
SEQ ID NO:25 if the Yersinia-specific amplification oligomer set comprises first and second Yersinia-specific amplification oligomers respectively comprising target-hybridizing sequences substantially corresponding to the nucleotide sequences of SEQ ID NO:60 and SEQ ID NO:45: or
SEQ ID NO:18 if the Yersinia-specific amplification oligomer set comprises first and second Yersinia-specific amplification oligomers respectively comprising target-hybridizing sequences substantially corresponding to the nucleotide sequences of SEQ ID NO:65 and SEQ ID NO:54.

Embodiment 52. The method of any one of Embodiments 48 to 51, wherein the sample is contacted with the Vibrio-specific amplification oligomer set.

Embodiment 53. The method of Embodiment 52, wherein the Vibrio-specific amplification oligomer set comprises the first and second V. parahaemolyticus-specific amplification oligomers and any V. parahaemolyticus target nucleic acid present in the sample is used as a template for generating a V. parahaemolyticus amplification product corresponding to the V. parahaemolyticus target region.

Embodiment 54. The method of Embodiment 53, wherein the detecting step (3) comprises contacting the in vitro nucleic acid amplification reaction with a V. parahaemolyticus-specific detection probe configured to specifically hybridize to the V. parahaemolyticus amplification product.

Embodiment 55. The method of Embodiment 54, wherein the V. parahaemolyticus-specific detection probe comprises a target-hybridizing sequence substantially corresponding to the nucleotide sequence of
SEQ ID NO:33, SEQ ID NO:89, SEQ ID NO:91, or SEQ ID NO:121 if the V. parahaemolyticus-specific amplification oligomer set comprises first and second V. parahaemolyticus-specific amplification oligomers respectively comprising target-hybridizing sequences substantially corresponding to the nucleotide sequences of (i) SEQ ID NO:51 and SEQ ID NO:76, (ii) SEQ ID NO:53 and SEQ ID NO:75, (iii) SEQ ID NO:52 and SEQ ID NO:75, (iv) SEQ ID NO:51 and SEQ ID NO:75, (v) SEQ ID NO:52 and SEQ ID NO:76, or (vi) SEQ ID NO:53 and SEQ ID NO:76; or
SEQ ID NO:15 if the V. parahaemolyticus-specific amplification oligomer set comprises first and second V. parahaemolyticus-specific amplification oligomers respectively comprising target-hybridizing sequences substantially corresponding to the nucleotide sequences of (i) SEQ ID NO:58 and SEQ ID NO:21, (ii) SEQ ID NO:58 and SEQ ID NO:86, or (iii) SEQ ID NO:58 and SEQ ID NO:20.

Embodiment 56. The method of any one of Embodiments 52 to 55, wherein the Vibrio-specific amplification oligomer set comprises the first and second V. vulnificus-specific amplification oligomers and any V. vulnificus target nucleic acid present in the sample is used as a template for generating a V. vulnificus amplification product corresponding to the V. vulnificus target region.

Embodiment 57. The method of Embodiment 56, wherein the detecting step (3) comprises contacting the in vitro nucleic acid amplification reaction with a V. vulnificus-specific detection probe configured to specifically hybridize to the V. vulnificus amplification product.

Embodiment 58. The method of Embodiment 57, wherein the V. vulnificus-specific detection probe comprises a target-hybridizing sequence substantially corresponding to the nucleotide sequence of
SEQ ID NO:9 if the V. vulnificus-specific amplification oligomer set comprises first and second V. vulnificus-specific amplification oligomers respectively comprising target-hybridizing sequences substantially corresponding to the nucleotide sequences of (i) SEQ ID NO:66 and SEQ ID NO:62, (ii) SEQ ID NO:42 and SEQ ID NO:37, (iii) SEQ ID NO:55 and SEQ ID NO:37, (iv) SEQ ID NO:42 and SEQ ID NO:84, (v) SEQ ID NO:42 and SEQ ID NO:47, (vi) SEQ ID NO:55 and SEQ ID NO:84, (vii) SEQ ID NO:55 and SEQ ID NO:47, or (viii) SEQ ID NO:55 and SEQ ID NO:5; or
SEQ ID NO:31 if the V. vulnificus-specific amplification oligomer set comprises first and second V. vulnificus-specific amplification oligomers respectively comprising target-hybridizing sequences substantially corresponding to the nucleotide sequences of (i) SEQ ID NO:58 and SEQ ID NO:21, (ii) SEQ ID NO:58 and SEQ ID NO:20, (iii) or (ii) SEQ ID NO:43 and SEQ ID NO:78.

Embodiment 59. The method of any one of Embodiments 52 to 58, wherein the Vibrio-specific amplification oligomer set comprises the first and second V. cholerae-specific amplification oligomers and any V. cholerae target nucleic acid present in the sample is used as a template for generating a V. cholerae amplification product corresponding to the V. cholerae target region.

Embodiment 60. The method of Embodiment 59, wherein the detecting step (3) comprises contacting the in vitro nucleic acid amplification reaction with a V. cholerae-specific detection probe configured to specifically hybridize to the V. cholerae amplification product.

Embodiment 61. The method of Embodiment 60, wherein the V. cholerae-specific detection probe comprises a target-hybridizing sequence substantially corresponding to the nucleotide sequence of
SEQ ID NO:12 if the V. cholerae-specific amplification oligomer set comprises first and second V. cholerae-specific amplification oligomers respectively comprising target-hybridizing sequences substantially corresponding to the nucleotide sequences of SEQ ID NO:56 and SEQ ID NO:1;
SEQ ID NO:35 or SEQ ID NO:88 if the V. cholerae-specific amplification oligomer set comprises first and second V. cholerae-specific amplification oligomers respectively comprising target-hybridizing sequences substantially corresponding to the nucleotide sequences of (i) SEQ ID NO:32 and SEQ ID NO:3, (ii) SEQ ID NO:57 and SEQ ID NO:64, (iii) SEQ ID NO:32 and SEQ ID NO:105, (iv) SEQ ID NO:92 and SEQ ID NO:3; (v) SEQ ID NO:92 and SEQ ID NO:105, (vi) SEQ ID NO:85 and SEQ ID NO:3, or (vii) SEQ ID NO:85 and SEQ ID NO:105; or
SEQ ID NO:67 or SEQ ID NO:82 if the V. cholerae-specific amplification oligomer set comprises first and second V. cholerae-specific amplification oligomers respectively comprising target-hybridizing sequences substantially corresponding to the nucleotide sequences of SEQ ID NO:94 and SEQ ID NO:19.

Embodiment 62. The method of any one of Embodiments 48 to 61, wherein the sample is contacted with the STEC-specific amplification oligomer set and any STEC O157 target nucleic acid present in the sample is used as a template for generating a STEC O157 amplification product corresponding to the STEC O157 target region.

Embodiment 63. The method of Embodiment 62, wherein the detecting step (3) comprises contacting the in vitro nucleic acid amplification reaction with a STEC-specific detection probe configured to specifically hybridize to the STEC O157 amplification product.

Embodiment 64. The method of Embodiment 63, wherein the STEC-specific detection probe comprises a target-hybridizing sequence substantially corresponding to the nucleotide sequence of
SEQ ID NO:73, SEQ ID NO:74, SEQ ID NO:7, or SEQ ID NO:59 if the STEC-specific amplification oligomer set comprises first and second STEC-specific amplification oligomers respectively comprising target-hybridizing sequences substantially corresponding to the nucleotide sequences of (i) SEQ ID NO:70 and SEQ ID NO:16, (ii) SEQ ID NO:71 and SEQ ID NO:44, (iii) SEQ ID NO:71 and SEQ ID NO:16, (iv) SEQ ID NO:70 and SEQ ID NO:44, (v) SEQ ID NO:70 and SEQ ID NO:107. (vi) SEQ ID NO:70 and SEQ ID NO:87, (vii) SEQ ID NO:71 and SEQ ID NO:107, or (viii) SEQ ID NO:71 and SEQ ID NO:87;
SEQ ID NO:96 or SEQ ID NO:103 if the STEC-specific amplification oligomer set comprises first and second STEC-specific amplification oligomers respectively comprising target-hybridizing sequences substantially corresponding to the nucleotide sequences of SEQ ID NO:90 and SEQ ID NO:100; or
SEQ ID NO:99 or SEQ ID NO:104 if the STEC-specific amplification oligomer set comprises first and second STEC-specific amplification oligomers respectively comprising target-hybridizing sequences substantially corresponding to the nucleotide sequences of SEQ ID NO:93 and SEQ ID NO:101.

Embodiment 65. The method of any one of Embodiments 48 to 64, wherein the sample is contacted with the Plesiomonas-specific amplification oligomer set and any Plesiomonas shigelloides target nucleic acid present in the sample is used as a template for generating a Plesiomonas shigelloides amplification product corresponding to the Plesiomonas shigelloides target region.

Embodiment 66. The method of Embodiment 65, wherein the detecting step (3) comprises contacting the in vitro nucleic acid amplification reaction with a Plesiomonas-specific detection probe configured to specifically hybridize to the Plesiomonas shigelloides amplification product.

Embodiment 67. The method of Embodiment 66, wherein the Plesiomonas-specific detection probe comprises a target-hybridizing sequence substantially corresponding to the nucleotide sequence of
SEQ ID NO:48 if the Plesiomonas-specific amplification oligomer set comprises first and second Plesiomonas-specific amplification oligomers respectively comprising target-hybridizing sequences substantially corresponding to the nucleotide sequences of (i) SEQ ID NO:2 and SEQ ID NO:49 or (ii) SEQ ID NO:17 and SEQ ID NO:98;
SEQ ID NO:80, SEQ ID NO:72, or SEQ ID NO:61 if the Plesiomonas-specific amplification oligomer set comprises first and second Plesiomonas-specific amplification oligomers respectively comprising target-hybridizing sequences substantially corresponding to the nucleotide sequences of (i) SEQ ID NO:95 and SEQ ID NO:1, (ii) SEQ ID NO:17 and SEQ ID NO:1, (iii) SEQ ID NO:95 and SEQ ID NO:106. (iv) SEQ ID NO:17 and SEQ ID NO:106, (v) SEQ ID NO: 17 and SEQ ID NO:98, or (vi) SEQ ID NO:95 and SEQ ID NO:98; or
SEQ ID NO:8 if the Plesiomonas-specific amplification oligomer set comprises first and second Plesiomonas-specific amplification oligomers respectively comprising target-hybridizing sequences substantially corresponding to the nucleotide sequences of (i) SEQ ID NO:79 and SEQ ID NO:81 or (ii) SEQ ID NO:79 and SEQ ID NO:68.

Embodiment 68. The method of any one of Embodiments 48 to 67, wherein the method is a multiplex method for detecting the presence or absence of at least two of Yersinia enterocolitica. Vibrio spp., Shiga Toxin Expressing E. coli (STEC) O157, and Plesiomonas shigelloides.

Embodiment 69. The method of any one of Embodiments 48 to 67, wherein the method is a multiplex method for detecting the presence or absence of at least three of Yersinia enterocolitica, Vibrio spp., Shiga Toxin Expressing E. coli (STEC) O157, and Plesiomonas shigelloides.

Embodiment 70. The method of any one of Embodiments 48 to 67, wherein the method is a multiplex method for detecting the presence or absence of each of Yersinia enterocolitica, Vibrio spp., Shiga Toxin Expressing E. coli (STEC) O157, and Plesiomonas shigelloides.

Embodiment 71. The method of Embodiment 48, wherein the method is a multiplex method for detecting the presence or absence of each of Yersinia enterocolitica, Vibrio spp., Shiga Toxin Expressing E. coli (STEC) O157, and Plesiomonas shigelloides.

Embodiment 72. The method of Embodiment 71, wherein the Yersinia-specific amplification oligomer set comprises first and second Yersinia-specific amplification oligomers respectively comprising target-hybridizing sequences substantially corresponding to the nucleotide sequences of SEQ ID NO:26 and SEQ ID NO:46.

Embodiment 73. The method of Embodiment 72, wherein the first Yersinia-specific amplification oligomer comprises a target-hybridizing sequence as shown in SEQ ID NO:26, including from 0 to 16 nucleotide analogs; and/or the second Yersinia-specific amplification oligomer comprises a target-hybridizing sequence as shown in SEQ ID NO:46, including from 0 to 16 nucleotide analogs.

Embodiment 74. The composition or kit of Embodiment 72 to 73, wherein the detecting step (3) comprises contacting the in vitro nucleic acid amplification reaction with a Yersinia-specific detection probe comprising a target-hybridizing sequence as shown in SEQ ID NO:40, including from 0 to 16 nucleotide analogs.

Embodiment 75. The method of any one of Embodiments 71 to 74, wherein the Vibrio-specific amplification oligomer set comprises first and second V. parahaemolyticus-specific amplification oligomers respectively comprising target-hybridizing sequences substantially corresponding to the nucleotide sequences of SEQ ID NO:51 and SEQ ID NO:76.

Embodiment 76. The method of Embodiment 75, wherein the first V parahaemolyticus-specific amplification oligomer comprises a target-hybridizing sequence as shown in SEQ ID NO:51, including from 0 to 16 nucleotide analogs; and/or the second V. parahaemolyticus-specific amplification oligomer comprises a target-hybridizing sequence as shown in SEQ ID NO:76, including from 0 to 16 nucleotide analogs.

Embodiment 77. The method of Embodiment 75 or 76, wherein the detecting step (3) comprises contacting the in vitro nucleic acid amplification reaction with a V. parahaemolyticus-specific detection probe comprising a target-hybridizing sequence as shown in SEQ ID NO:33, SEQ ID NO:89, SEQ ID NO:91, or SEQ ID NO:121, including from 0 to 16 nucleotide analogs.

Embodiment 78. The method of any one of Embodiments 71 to 77, wherein the Vibrio-specific amplification oligomer set comprises first and second V. vulnificus-specific amplification oligomers respectively comprising target-hybridizing sequences substantially corresponding to the nucleotide sequences of SEQ ID NO:66 and SEQ ID NO:62.

Embodiment 79. The method of Embodiment 78, wherein the first V. vulnificus-specific amplification oligomer comprises a target-hybridizing sequence as shown in SEQ ID NO:66, including from 0 to 16 nucleotide analogs; and/or the second V. vulnificus-specific amplification oligomer comprises a target-hybridizing sequence as shown in SEQ ID NO:62, including from 0 to 16 nucleotide analogs.

Embodiment 80. The method of Embodiment 78 or 79, wherein the detecting step (3) comprises contacting the in vitro nucleic acid amplification reaction with a V. vulnificus-specific detection probe comprising a target-hybridizing sequence as shown in SEQ ID NO:9, including from 0 to 16 nucleotide analogs.

Embodiment 81. The method of any one of Embodiments 71 to 80, wherein the Vibrio-specific amplification oligomer set comprises first and second V. cholerae-specific amplification oligomers respectively comprising target-hybridizing sequences substantially corresponding to the nucleotide sequences of SEQ ID NO:56 and SEQ ID NO:11.

Embodiment 82. The method of Embodiment 81, wherein the first V. cholerae-specific amplification oligomer comprises a target-hybridizing sequence as shown in SEQ ID NO:56, including from 0 to 16 nucleotide analogs; and/or the second V. cholerae-specific amplification oligomer comprises a target-hybridizing sequence as shown in SEQ ID NO: 11, including from 0 to 16 nucleotide analogs.

Embodiment 83. The method of Embodiment 81 or 82, wherein the detecting step (3) comprises contacting the in vitro nucleic acid amplification reaction with a V. cholerae-specific detection probe comprising a target-hybridizing sequence as shown in SEQ ID NO:12, including from 0 to 16 nucleotide analogs.

Embodiment 84. The method of any one of Embodiments 71 to 83, wherein the STEC-specific amplification oligomer set comprises first and second STEC-specific amplification oligomers respectively comprising target-hybridizing sequences substantially corresponding to the nucleotide sequences of SEQ ID NO:70 and SEQ ID NO:16.

Embodiment 85. The method of Embodiment 84, wherein the first STEC-specific amplification oligomer comprises a target-hybridizing sequence as shown in SEQ ID NO:70, including from 0 to 16 nucleotide analogs; and/or the second STEC-specific amplification oligomer comprises a target-hybridizing sequence as shown in SEQ ID NO:16, including from 0 to 16 nucleotide analogs.

Embodiment 86. The method of Embodiment 84 or 85, wherein the detecting step (3) comprises contacting the in vitro nucleic acid amplification reaction with a STEC-specific detection probe comprising a target-hybridizing sequence as shown in SEQ ID NO:73, SEQ ID NO:74, SEQ ID NO:7, or SEQ ID NO:59, including from 0 to 16 nucleotide analogs.

Embodiment 87. The method of any one of Embodiments 71 to 86, wherein the Plesiomonas-specific amplification oligomer set comprises first and second Plesiomonas-specific amplification oligomers respectively comprising target-hybridizing sequences substantially corresponding to the nucleotide sequences of SEQ ID NO:2 and SEQ ID NO:49.

Embodiment 88. The method of Embodiment 87, wherein the first Plesiomonas-specific amplification oligomer comprises a target-hybridizing sequence as shown in SEQ ID NO:2, including from 0 to 16 nucleotide analogs; and/or the second Plesiomonas-specific amplification oligomer comprises a target-hybridizing sequence as shown in SEQ ID NO:49, including from 0 to 16 nucleotide analogs.

Embodiment 89. The method of Embodiment 87 or 88, wherein the detecting step (3) comprises contacting the in vitro nucleic acid amplification reaction with a Plesiomonas-specific detection probe comprising a target-hybridizing sequence as shown in SEQ ID NO:48, including from 0 to 16 nucleotide analogs.

Embodiment 90. The method of any one of Embodiments 50, 51, 54, 55, 57, 58, 60, 61, 63, 64, 66, 67, 74, 77, 80, 83, 86, and 89, wherein one or more of the detection probes comprises a detectable label.

Embodiment 91. The method of Embodiment 90, wherein the detectable label is a fluorescent or chemiluminescent label.

Embodiment 92. The method of Embodiment 90, wherein the detectable label is a fluorescent label and each of the one or more detection probes further comprises a non-fluorescent quencher.

Embodiment 93. The method of any one of Embodiments 48 to 92, wherein the sample is a human sample.

Embodiment 894. The method of any one of Embodiments 48 to 93, wherein the sample is a stool sample or a blood sample.

Embodiment 95. A method for synthesizing an oligonucleotide, comprising the steps of:
(a) obtaining a solid support comprising at least one nucleobase residue, wherein the at least one nucleobase residue is covalently bound at a 3′ position to the solid support;
(b) coupling a 5′ position of the nucleobase residue furthest from the solid support to a 3′ position of another nucleobase residue;
(c) repeating step (b) at least 16 additional times, thereby generating at least 18 contiguous nucleobase residues coupled to the solid support; and
(d) cleaving the at least 18 contiguous nucleobase residues generated in step (c), thereby obtaining the oligonucleotide,
wherein the oligonucleotide comprises a nucleotide sequence selected from the group consisting of SEQ ID NOs:1-13, 15-22, 24-35, 37, 40-68, and 70-107, including from 0 to 16 nucleotide analogs.

Embodiment 96. The method of Embodiment 95, wherein the nucleotide sequence is selected from the group consisting of SEQ ID NOs:108-135.

Embodiment 97. A method for synthesizing a pair of oligonucleotides, comprising synthesizing a first oligonucleotide and synthesizing a second oligonucleotide,
wherein each of the synthesizing the first oligonucleotide and the synthesizing the second oligonucleotide comprises the steps of:
(a) obtaining a solid support comprising at least one nucleobase residue, wherein the at least one nucleobase residue is covalently bound at a 3′ position to the solid support;
(b) coupling a 5′ position of the nucleobase residue furthest from the solid support to a 3′ position of another nucleobase residue;
(c) repeating step (b) at least 16 additional times, thereby generating at least 18 contiguous nucleobase residues coupled to the solid support; and
(d) cleaving the at least 18 contiguous nucleobase residues generated in step (c), thereby obtaining the oligonucleotide, and
wherein the first oligonucleotide and the second oligonucleotide respectively comprise the nucleotide sequences of any one of
   SEQ ID NO:26 and SEQ ID NO:46, including from 0 to 16 nucleotide analogs;
   SEQ ID NO:97 and SEQ ID NO:4, including from 0 to 16 nucleotide analogs;
   SEQ ID NO:102 and SEQ ID NO:24, including from 0 to 16 nucleotide analogs;
   SEQ ID NO:6 and SEQ ID NO:24, including from 0 to 16 nucleotide analogs;
   SEQ ID NO:13 and SEQ ID NO:22, including from 0 to 16 nucleotide analogs;
   SEQ ID NO:77 and SEQ ID NO:22, including from 0 to 16 nucleotide analogs;
   SEQ ID NO:77 and SEQ ID NO:41, including from 0 to 16 nucleotide analogs;
   SEQ ID NO:28 and SEQ ID NO:30, including from 0 to 16 nucleotide analogs;
   SEQ ID NO:60 and SEQ ID NO:45, including from 0 to 16 nucleotide analogs;
   SEQ ID NO:65 and SEQ ID NO:54, including from 0 to 16 nucleotide analogs;
   SEQ ID NO:10 and SEQ ID NO:4, including from 0 to 16 nucleotide analogs;
   SEQ ID NO:51 and SEQ ID NO:76, including from 0 to 16 nucleotide analogs;
   SEQ ID NO:58 and SEQ ID NO:21, including from 0 to 16 nucleotide analogs;
   SEQ ID NO:58 and SEQ ID NO:86, including from 0 to 16 nucleotide analogs;
   SEQ ID NO:53 and SEQ ID NO:75, including from 0 to 16 nucleotide analogs;
   SEQ ID NO:58 and SEQ ID NO:20, including from 0 to 16 nucleotide analogs;
   SEQ ID NO:52 and SEQ ID NO:75, including from 0 to 16 nucleotide analogs;
   SEQ ID NO:51 and SEQ ID NO:75, including from 0 to 16 nucleotide analogs;
   SEQ ID NO:52 and SEQ ID NO:76, including from 0 to 16 nucleotide analogs;
   SEQ ID NO:53 and SEQ ID NO:76, including from 0 to 16 nucleotide analogs;
   SEQ ID NO:66 and SEQ ID NO:62, including from 0 to 16 nucleotide analogs;
   SEQ ID NO:42 and SEQ ID NO:37, including from 0 to 16 nucleotide analogs;
   SEQ ID NO:55 and SEQ ID NO:37, including from 0 to 16 nucleotide analogs;
   SEQ ID NO:43 and SEQ ID NO:78, including from 0 to 16 nucleotide analogs;
   SEQ ID NO:58 and SEQ ID NO:20, including from 0 to 16 nucleotide analogs;
   SEQ ID NO:42 and SEQ ID NO:84, including from 0 to 16 nucleotide analogs;
   SEQ ID NO:42 and SEQ ID NO:47, including from 0 to 16 nucleotide analogs;
   SEQ ID NO:55 and SEQ ID NO:84, including from 0 to 16 nucleotide analogs;
   SEQ ID NO:55 and SEQ ID NO:47, including from 0 to 16 nucleotide analogs;
   SEQ ID NO:55 and SEQ ID NO:5, including from 0 to 16 nucleotide analogs;
   SEQ ID NO:56 and SEQ ID NO: 11, including from 0 to 16 nucleotide analogs;
   SEQ ID NO:32 and SEQ ID NO:3, including from 0 to 16 nucleotide analogs;
   SEQ ID NO:57 and SEQ ID NO:64, including from 0 to 16 nucleotide analogs;
   SEQ ID NO:94 and SEQ ID NO:19, including from 0 to 16 nucleotide analogs;
   SEQ ID NO:32 and SEQ ID NO:105, including from 0 to 16 nucleotide analogs;
   SEQ ID NO:92 and SEQ ID NO:3, including from 0 to 16 nucleotide analogs;
   SEQ ID NO:92 and SEQ ID NO:105, including from 0 to 16 nucleotide analogs;
   SEQ ID NO:85 and SEQ ID NO:3, including from 0 to 16 nucleotide analogs;
   SEQ ID NO:85 and SEQ ID NO:105, including from 0 to 16 nucleotide analogs;
   SEQ ID NO:70 and SEQ ID NO:16, including from 0 to 16 nucleotide analogs;
   SEQ ID NO:93 and SEQ ID NO:101, including from 0 to 16 nucleotide analogs;
   SEQ ID NO:71 and SEQ ID NO:44, including from 0 to 16 nucleotide analogs;
   SEQ ID NO:90 and SEQ ID NO:100, including from 0 to 16 nucleotide analogs;
   SEQ ID NO:71 and SEQ ID NO:16, including from 0 to 16 nucleotide analogs;
   SEQ ID NO:70 and SEQ ID NO:44, including from 0 to 16 nucleotide analogs;
   SEQ ID NO:70 and SEQ ID NO:107, including from 0 to 16 nucleotide analogs;
   SEQ ID NO:70 and SEQ ID NO:87, including from 0 to 16 nucleotide analogs;
   SEQ ID NO:71 and SEQ ID NO:107, including from 0 to 16 nucleotide analogs;
   SEQ ID NO:71 and SEQ ID NO:87, including from 0 to 16 nucleotide analogs;
   SEQ ID NO:2 and SEQ ID NO:49, including from 0 to 16 nucleotide analogs;
   SEQ ID NO:95 and SEQ ID NO:1, including from 0 to 16 nucleotide analogs;
   SEQ ID NO:79 and SEQ ID NO:81, including from 0 to 16 nucleotide analogs;
   SEQ ID NO:17 and SEQ ID NO:1, including from 0 to 16 nucleotide analogs;
   SEQ ID NO:95 and SEQ ID NO:106, including from 0 to 16 nucleotide analogs;
   SEQ ID NO:17 and SEQ ID NO:106, including from 0 to 16 nucleotide analogs;
   SEQ ID NO:79 and SEQ ID NO:68, including from 0 to 16 nucleotide analogs;
   SEQ ID NO:17 and SEQ ID NO:98, including from 0 to 16 nucleotide analogs; or
   SEQ ID NO:95 and SEQ ID NO:98, including from 0 to 16 nucleotide analogs.

Embodiment 98. The composition or kit of any one of Embodiments 20, 23, 26, 29, 32, and 35, wherein one or more of the detection probes comprises a blocking moiety at or near the 3′ terminus.

Embodiment 99. The composition or kit of claim 98, wherein the blocking moiety is an inverted nucleotide.

Embodiment 100. The composition or kit of claim 99, wherein the inverted nucleotide is an inverted C nucleotide.

Embodiment 101. The method of any one of Embodiments 50, 51, 54, 55, 57, 58, 60, 61, 63, 64, 66, 67, 74, 77, 80, 83, 86, and 89, wherein one or more of the detection probes comprises a blocking moiety at or near the 3′ terminus.

Embodiment 102. The method of claim 101, wherein the blocking moiety is an inverted nucleotide.

Embodiment 103. The method of claim 102, wherein the inverted nucleotide is an inverted C nucleotide.

These and other aspects and embodiments will become evident upon reference to the following detailed description.

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise. For example, “a nucleic acid” as used herein is understood to represent one or more nucleic acids. As such, the terms “a” (or “an”), “one or more,” and “at least one” can be used interchangeably herein.

When a value is expressed as “about” X or “approximately” X, the stated value of X will be understood to be accurate to ±10%.

All ranges are to be interpreted as encompassing the endpoints in the absence of express exclusions such as “not including the endpoints”; thus, for example, “from 0 to 16” includes the values 0 and 16.

“Sample” includes any specimen that may contain Yersinia enterocolitica, Vibrio spp., STEC O157, and/or Plesiomonas shigelloides, including components thereof, such as nucleic acids or fragments of nucleic acids. Samples include “biological samples” which include any tissue or material derived from a living or dead human, including, for example, stool, blood, plasma, serum, blood cells, saliva, mucous, and cerebrospinal fluid. The biological sample may be treated to physically or mechanically disrupt tissue or cell structure, thus releasing intracellular components into a solution which may further contain enzymes, buffers, salts, detergents, and the like, which are used to prepare a biological sample for analysis. Also, samples may include processed samples such as samples in which one or more components have been concentrated or purified. Processed samples include, e.g., those obtained from passing samples over or through a filtering device, or following centrifugation, or by adherence to a medium, matrix, or support.

A “nucleotide” as used herein is a subunit of a nucleic acid consisting of a phosphate group, a 5-carbon sugar, and a nitrogenous base (also referred to herein as “nucleobase”). The 5-carbon sugar found in RNA is ribose. In DNA, the 5-carbon sugar is 2′-deoxyribose.

“Nucleic acid” and “polynucleotide” refer to a multimeric compound comprising nucleotides and/or nucleotide analogs linked together to form a biopolymer. The biopolymers include conventional RNA, conventional DNA, mixed RNA-DNA, and nucleotide-analog-containing versions thereof. A nucleic acid “backbone” may be made up of a variety of linkages, including one or more of sugar-phosphodiester linkages, peptide-nucleic acid bonds (“peptide nucleic acids” or PNA), phosphorothioate linkages, methylphosphonate linkages, or combinations thereof. Sugar moieties of a nucleic acid may be ribose, deoxyribose, or similar compounds with substitutions. e.g., analogs with a methoxy, fluoro or halide group at the 2′ position of the ribose (also referred to herein as “2′-O-Me” or “2′-methoxy” or 2′-fluoro, or “2′-halide”). Nitrogenous bases may be conventional bases, adenine (A), uracil (U), guanine (G), thymine (T), and cytosine (C), and analogs thereof (e.g., inosine, 5 methyl 2′ deoyxcytosine (“5-methyl cytosine”) (5mC), isoguanine, propyne dC (pdC), or propyne dU (pdU)). As used in the present disclosure, pdC is considered a cytosine analogue; and pdU is considered a thymine analogue. Nucleic acids may include one or more “abasic” residues where the backbone includes no nitrogenous base for position(s) of the polymer.

By “RNA and DNA equivalents” is meant RNA and DNA molecules having essentially the same complementary base pair hybridization properties. RNA and DNA equivalents have different sugar moieties (i.e., ribose versus deoxyribose) and may differ by the presence of uracil in RNA and thymine in DNA. The differences between RNA and DNA equivalents do not contribute to differences in homology because the equivalents have the same degree of complementarity to a particular sequence. By “DNA/RNA chimeric” is meant a nucleic acid comprising both DNA and RNA nucleotides. One example of a DNA/RNA chimeric is a DNA oligomer wherein all thymine (T) nucleobase residues are replaced with uracil (U). Unless the context clearly dictates otherwise, reference to a Yersinia enterocolitica, Vibrio spp., STEC O157, or Plesiomonas shigelloides nucleic acid includes the RNA and DNA equivalents and DNA/RNA chimerics thereof.

The phrase “including from 0 to 16 nucleotide analogs.” as used herein following reference to one or more nucleotide sequences by SEQ ID NO. means that the referenced sequence(s) include equivalents of each sequence having from 0 to 16 nucleotide analogs. By “equivalents having from 0 to 16 nucleotide analogs” is meant oligonucleotides that (i) have from 0 to 16 nucleotide analogs substituting conventional nucleotides within the reference sequence and (ii) have essentially the same complementary base pair hybridization properties as the reference sequence.

“Oligomer,” “oligonucleotide,” or “oligo” refers to a nucleic acid of generally less than 1,000 nucleotides (nt), including those in a size range having a lower limit of about 5 nt and an upper limit of about 500 to 900 nt. Some particular embodiments are oligonucleotides in a size range with a lower limit of about 5 to 15, 16, 17, 18, 19, or 20 nt and an upper limit of about 50 to 600 nt, and other particular embodiments are in a size range with a lower limit of about 10 to 20 nt and an upper limit of about 22 to 100 nt. Oligonucleotides may be purified from naturally occurring sources but may be synthesized by using any well-known enzymatic or chemical method. Oligomers may be referred to by a functional name (e.g., detection probe, primer, or promoter primer) but those skilled in the art will understand that such terms refer to oligomers.

A “target nucleic acid” as used herein is a nucleic acid comprising a target sequence to be amplified. Target nucleic acids may be DNA or RNA and may be either single-stranded or double-stranded. The target nucleic acid may include other sequences besides the target sequence, which may not be amplified.

The term “target region” or “target nucleic acid region” as used herein refers to the particular nucleotide sequence of the target nucleic acid that is to be amplified and/or detected. The “target region” includes the complexing sequences to which oligonucleotides (e.g., priming oligonucleotides and/or promoter oligonucleotides) complex during an amplification processes (e.g., PCR, TMA). Unless the context clearly dictates otherwise, where the target nucleic acid is originally single-stranded, the term “target region” will also refer to the sequence complementary to the “target region” as present in the target nucleic acid, and where the target nucleic acid is originally double-stranded, the term “target region” refers to both the sense (+) and antisense (−) strands.

The term “target sequence” or “target nucleic acid sequence” as used herein refers to the particular nucleotide sequence of the target nucleic acid to which oligonucleotides (e.g., priming oligonucleotides, detection probes, or capture probes) complex during amplification and/or detection of the target nucleic acid.

“Target-hybridizing sequence” or “target-specific sequence” is used herein to refer to the portion of an oligomer that is configured to hybridize with a target nucleic acid sequence. Preferably, the target-hybridizing sequences are configured to specifically hybridize with a target nucleic acid sequence. Target-hybridizing sequences may be 100% complementary to the portion of the target sequence to which they are configured to hybridize, but not necessarily. Target-hybridizing sequences may also include inserted, deleted and/or substituted nucleotide residues relative to a target sequence.

“Non-target-specific sequence” or “non-target-hybridizing sequence” as used herein refers to a region of an oligomer sequence, wherein said region does not stably hybridize with a target sequence under standard hybridization conditions. Oligomers with non-target-specific sequences include, but are not limited to, promoter primers, promoter providers, target capture oligomers, torches, and molecular beacons.

The term “target a sequence,” as used herein in reference to a region of a Yersinia enterocolitica, Vibrio spp., STEC O157, or Plesiomonas shigelloides nucleic acid, refers to a process whereby an oligonucleotide hybridizes to a target region in a manner that allows for amplification and detection as described herein. In one embodiment, the oligonucleotide is complementary with the targeted Yersinia enterocolitica, Vibrio spp., STEC O157, or Plesiomonas shigelloides nucleic acid sequence and contains no mismatches. In another embodiment, the oligonucleotide is complementary but contains 1, 2, 3, 4, or 5 mismatches with the targeted Yersinia enterocolitica, Vibrio spp., STEC O157, or Plesiomonas shigelloides nucleic acid sequence.

The term “configured to” denotes an actual arrangement of the polynucleotide sequence configuration of a referenced oligonucleotide target-hybridizing sequence. For example, amplification oligomers that are configured to generate a specified amplicon from a target nucleic acid region have polynucleotide sequences that hybridize to the target region and can be used in an amplification reaction to generate the amplicon. Also, as an example, oligonucleotides that are configured to specifically hybridize to a target region have a polynucleotide sequence that specifically hybridizes to the referenced sequence under stringent hybridization conditions.

The term “configured to specifically hybridize to” as used herein means that the target-hybridizing region of an amplification oligonucleotide, detection probe, or other oligonucleotide is designed to have a polynucleotide sequence that could target a sequence of the referenced Yersinia enterocolitica, Vibrio spp., STEC O157, or Plesiomonas shigelloides target region. The oligonucleotide is designed to function as a component of an assay for amplification and detection of Yersinia enterocolitica. Vibrio spp., STEC O157, or Plesiomonas shigelloides target nucleic acid from a sample, and therefore is designed to target Yersinia enterocolitica, Vibrio spp., STEC O157, or Plesiomonas shigelloides nucleic acid in the presence of other nucleic acids commonly found in testing samples. “Specifically hybridize to” does not mean exclusively hybridize to, as some small level of hybridization to non-target nucleic acids may occur, as is understood in the art. Rather, “specifically hybridize to” means that the oligonucleotide is configured to function in an assay to primarily hybridize the target so that an accurate detection of target nucleic acid in a sample can be determined.

An “amplification oligonucleotide” or “amplification oligomer” is an oligonucleotide that hybridizes to a target nucleic acid and participates in a nucleic acid amplification reaction. e.g., serving as a primer. Amplification oligomers can have 3′ ends that are extended by polymerization as part of the nucleic acid amplification reaction. Amplification oligomers can alternatively have 3′ ends that are not extended by polymerization, but provide a component that facilitates nucleic acid amplification, e.g., a promoter sequence joined 5′ to the target hybridizing sequence of the amplification oligomer. Such an amplification oligomer is referred to as a promoter provider. Amplification oligomers that provide both a 3′ target hybridizing region that is extendable by polymerization and a 5′ promoter sequence are referred to as promoter primers. Amplification oligomers may be optionally modified to include 5′ non-target hybridizing regions such as tags, promoters (as mentioned), or other sequences used or useful for manipulating or amplifying the primer or target oligonucleotide.

“Nucleic acid amplification” refers to any in vitro procedure that produces multiple copies of a target nucleic acid sequence, or its complementary sequence, or fragments thereof (i.e., an amplified sequence containing less than the complete target nucleic acid). Examples of nucleic acid amplification procedures include transcription associated methods, such as transcription-mediated amplification (TMA), nucleic acid sequence-based amplification (NASBA) and others (e.g., U.S. Pat. Nos. 5,399,491, 5,554,516, 5,437,990, 5,130,238, 4,868,105, and 5,124,246), and polymerase chain reaction (PCR) (e.g., U.S. Pat. Nos. 4,683,195, 4,683,202, and 4,800,159).

By “amplicon” or “amplification product” is meant a nucleic acid molecule generated in a nucleic acid amplification reaction and which is derived from a target nucleic acid. An amplicon or amplification product contains a target nucleic acid region that may be of the same or opposite sense as the target nucleic acid.

As used herein, the term “relative fluorescence unit” (“RFU”) is a unit of measurement of fluorescence intensity. RFU varies with the characteristics of the detection means used for the measurement and can be used as a measurement to compare relative intensities between samples and controls.

“Detection probe oligomer,” “detection probe,” or “probe” refers to an oligomer that hybridizes specifically to a target nucleic acid region, including an amplified product, under conditions that promote nucleic acid hybridization, for detection of the target nucleic acid. Detection may either be direct (i.e., probe hybridized directly to the target) or indirect (i.e., a probe hybridized to an intermediate structure that links the probe to the target). A probe's target sequence generally refers to the specific sequence within a larger sequence which the probe hybridizes specifically. A detection probe may include target-specific sequence(s) and non-target-specific sequence(s). Such non-target-specific sequences can include sequences which will confer a desired secondary or tertiary structure, such as a hairpin structure, which can be used to facilitate detection and/or amplification.

As used herein, a nucleic acid “substantially corresponding to” a specified nucleic acid sequence, or its complement, means that the oligonucleotide is sufficiently similar to the reference nucleic acid sequence such that the oligonucleotide has similar hybridization properties to the reference nucleic acid sequence in that it would hybridize with the same target nucleic acid sequence under stringent hybridization conditions. Substantially corresponding nucleic acids vary by at least one nucleotide from the specified nucleic acid. This variation may be stated in terms of a percentage of sequence identity or complementarity between the nucleic acid and the specified nucleic acid. In some embodiments, a nucleic acid “substantially corresponding to” a reference sequence has from about 80% to 100% nucleobase sequence identity or complementarity to the reference sequence; in preferred embodiments, the percentage is from about 85% to 100%, more preferably from about 90% to 100% or from about 95% to 100%. One skilled in the art will understand that the recited ranges include all whole and rational numbers of the range (e.g., 92%. 92.377%, etc.).

By “stringent hybridization conditions,” or “stringent conditions” is meant conditions permitting an oligomer to preferentially hybridize to a target nucleic acid region and not to nucleic acid derived from a closely related non-target nucleic acid (i.e., conditions permitting an oligomer to hybridize to its target sequence to form a stable oligomer:target hybrid, but not form a sufficient number of stable oligomer:non-target hybrids, so as to allow for amplification and/or detection of target nucleic acids but not non-targeted organisms). While the definition of stringent hybridization conditions does not vary, the actual reaction environment that can be used for stringent hybridization may vary depending upon factors including the GC content and length of the oligomer, the degree of similarity between the oligomer sequence and sequences of non-target nucleic acids that may be present in the test sample, and the target sequence. Hybridization conditions include the temperature and the composition of the hybridization reagents or solutions. Stringent hybridization conditions are readily ascertained by those having ordinary skill in the art.

“Label” or “detectable label” refers to a moiety or compound joined directly or indirectly to a probe that is detected or leads to a detectable signal. Direct joining may use covalent bonds or non-covalent interactions (e.g., hydrogen bonding, hydrophobic or ionic interactions, and chelate or coordination complex formation) whereas indirect joining may use a bridging moiety or linker (e.g., via an antibody or additional oligonucleotide(s), which may amplify a detectable signal). Any detectable moiety may be used, e.g., radionuclide, ligand such as biotin or avidin, enzyme, enzyme substrate, reactive group, chromophore such as a dye or particle (e.g., latex or metal bead) that imparts a detectable color, luminescent compound (e.g., bioluminescent, phosphorescent, or chemiluminescent compound such as an acridinium ester (“AE”) compound), and fluorescent compound (i.e., fluorophore). Embodiments of fluorophores include those that absorb light in the range of about 495 to 690 nm and emit light in the range of about 520 to 705 nm, which include those known as FAM™. TET™. CAL FLUOR™ (Orange or Red), and QUASAR™ compounds. Fluorophores may be used in combination with a quencher molecule that absorbs light when in close proximity to the fluorophore to diminish background fluorescence. Such quenchers are well known in the art and include, e.g., BLACK HOLE QUENCHER™ (or BHQ™) or TAMRA™ compounds. Particular embodiments include a “homogeneous detectable label” that is detectable in a homogeneous system in which bound labeled probe in a mixture exhibits a detectable change compared to unbound labeled probe, which allows the label to be detected without physically removing hybridized from unhybridized labeled probe (e.g., U.S. Pat. Nos. 5,283,174, 5,656,207, and 5,658,737). Particular homogeneous detectable labels include chemiluminescent compounds, including acridinium ester (“AE”) compounds, such as standard AE or AE derivatives, which are well known (U.S. Pat. Nos. 5,656,207, 5,658,737, and 5,639,604). Methods of synthesizing labels, attaching labels to nucleic acid, and detecting signals from labels are well known (e.g., Sambrook et al., Molecular Cloning, A Laboratory Manual, 2nd ed. (Cold Spring Harbor Laboratory Press, Cold Spring Harbor, NY, 1989) at Chapt. 10, and U.S. Pat. Nos. 5,658,737, 5,656,207, 5,547,842, 5,283,174, and 4,581,333, and EP Pat. App. 0 747 706). Particular methods of linking an AE compound to a nucleic acid are known (e.g., U.S. Pat. Nos. 5,585,481 and 5,639,604, see column 10, line 6 to column 11, line 3, and Example 8). Particular AE labeling positions are a probe's central region and near a region of A/T base pairs, at a probe's 3′ or 5′ terminus, or at or near a mismatch site with a known sequence that is the probe should not detect compared to the desired target sequence. Other detectably labeled probes include, e.g., TaqMan™ probes, molecular torches, and molecular beacons. TaqMan™ probes include a donor and acceptor label wherein fluorescence is detected upon enzymatically degrading the probe during amplification in order to release the fluorophore from the presence of the quencher. Molecular torches and beacons exist in open and closed configurations wherein the closed configuration quenches the fluorophore and the open position separates the fluorophore from the quencher to allow fluorescence. Hybridization to target opens the otherwise closed probes.

A “non-extendable” oligomer includes a blocking moiety at or near its 3′-terminus to prevent extension. A blocking group near the 3′ end is in some embodiments within five residues of the 3′ end and is sufficiently large to limit binding of a polymerase to the oligomer. In other embodiments, a blocking group is covalently attached to the 3′ terminus. Suitable blocking groups include. e.g., alkyl groups, non-nucleotide linkers, alkane-diol dideoxynucleotide residues, cordycepin, 3′-deoxy nucleotides, 3′-phosphorylated nucleotides, inverted nucleotides (e.g., an inverted C nucleotide, also referred to herein as a “reverse polarity C”), proteins, peptides, and labels such as fluorophores or quenchers.

References, particularly in the embodiments, to “the sequence of SEQ ID NO:X” refer to the sequence of nucleotides and/or nucleotide analogs linked together to form a biopolymer. Reference to a sequence by SEQ ID NO does not connote the identity of the backbone (e.g., RNA, 2′-O-Me RNA, or DNA) or any nucleobase modifications (e.g., methylation of cytosine residues (“5MeC”)) unless the context clearly dictates otherwise. In some instances, the sequence of a SEQ ID NO is followed by the statement “including from [x-y]nucleotide analogs”: it is understood that the nucleotide analogs may be substitutions within the sequence of the SEQ ID NO. Unless the context clearly dictates otherwise, reference to a sequence by SEQ ID NO includes reference to its complementary sequence (e.g., reference to the sequence 5.-ttagc-3′ includes reference to the sequence 5′-gctaa-3′).

“Separating” or “purifying” means that one or more components of a sample are removed or separated from other sample components. Sample components include target nucleic acids usually in a generally aqueous solution phase, which may also include cellular fragments, proteins, carbohydrates, lipids, and other nucleic acids. “Separating” or “purifying” does not connote any degree of purification. Typically, separating or purifying removes at least 70%, or at least 80%, or at least 95% of the target nucleic acid from other sample components.

The term “non-linear surfactant,” as used herein, means a surfactant having a branched chain structure. A non-linear surfactant may include one or more ring structures, which may be, for example, in a principal chain and/or in one or more branched chains. Exemplary non-linear surfactants include polysorbate 20, polysorbate 40, polysorbate 60, and digitonin. In certain variations, the non-linear surfactant is non-ionic.

The term “specificity,” in the context of an amplification and/or detection system, is used herein to refer to the characteristic of the system which describes its ability to distinguish between target and non-target sequences dependent on sequence and assay conditions. In terms of nucleic acid amplification, specificity generally refers to the ratio of the number of specific amplicons produced to the number of side-products (e.g., the signal-to-noise ratio). In terms of detection, specificity generally refers to the ratio of signal produced from target nucleic acids to signal produced from non-target nucleic acids.

The term “sensitivity” is used herein to refer to the precision with which a nucleic acid amplification reaction can be detected or quantitated. The sensitivity of an amplification reaction is generally a measure of the smallest copy number of the target nucleic acid that can be reliably detected in the amplification system, and will depend, for example, on the detection assay being employed, and the specificity of the amplification reaction, e.g., the ratio of specific amplicons to side-products.

### DETAILED DESCRIPTION

Provided herein are compositions, kits, and methods for amplifying and/or detecting target nucleic acid from at least one enteric pathogen in a sample, wherein the at least one enteric pathogen is selected from Yersinia enterocolitica, Vibrio spp. (e.g., V. parahaemolyticus, V. vulnificus, and/or V. cholerae), Shiga Toxin Expressing E. coli (STEC) O157, and Plesiomonas shigelloides. Preferably, the samples are biological samples. The compositions, kits, and methods provide oligonucleotide sequences that target enteric pathogen gene sequences or their complementary sequences. Such oligonucleotides may be used as amplification oligonucleotides, which may include primers, promoter primers, blocked oligonucleotides, and promoter provider oligonucleotides, whose functions have been described previously (see, e.g., U.S. Pat. No. 4,683,195; 4,683,202; 4,800,159; 5,399,491; 5,554,516; 5,824,518; and 7,374,885; each incorporated by reference herein). Other oligonucleotides may be used as probes for detecting amplified sequences or for capture of an enteric pathogen target nucleic acid.

The methods provide for the sensitive and specific detection of Yersinia enterocolitica, Vibrio spp., STEC O157, and/or Plesiomonas shigelloides nucleic acids. The methods include performing nucleic acid amplification of a target region of one or more of Yersinia enterocolitica, Vibrio spp., STEC O157, and Plesiomonas shigelloides, and detecting one or more amplified products by, for example, specifically hybridizing the amplified product(s) with one or more nucleic acid detection probes that provide a signal to indicate the presence of the at least one enteric pathogen in the sample. The amplification step includes contacting the sample with (a) one or more Yersinia-specific amplification oligomers specific for a target sequence in a Yersinia enterocolitica target nucleic acid, (b) one or more Vibrio-specific amplification oligomers specific for a target sequence in a Vibrio spp. target nucleic acid, (c) one or more STEC-specific amplification oligomers specific for a target sequence in a STEC O157 target nucleic acid, and/or (d) one or more Plesiomonas-specific amplification oligomers specific for a target sequence in a Plesiomonas shigelloides target nucleic acid. Particularly suitable target nucleic acids include the ail, invA, and ystA genes of Yersinia enterocolitica, the gyrB gene of Vibrio parahaemolyticus and Vibrio vulnificus, the ompW and toxR genes of Vibrio cholerae, the rfbE gene of STEC O157, and/or the hugA gene of Plesiomonas shigelloides. Nucleic acid amplification is performed to produce one or more amplification products corresponding to one or more of the Yersinia enterocolitica, Vibrio spp., STEC O157, and/or Plesiomonas shigelloides target nucleic acids, if present in the sample, wherein the amplification reaction synthesizes additional copies of the target sequence or its complement by using at least one nucleic acid polymerase and the one or more amplification oligomers to produce the copies from a template strand (e.g., by extending the sequence from a primer using the template strand). One embodiment for detecting the amplification product uses a hybridizing step that includes contacting the amplified product with at least one detection probe oligomer specific for a sequence amplified by the selected amplification oligomers, e.g., a sequence contained in the target sequence flanked by a pair of selected amplification oligomers.

In some aspects, oligonucleotides are provided, e.g., in a kit or composition. Oligonucleotides generally comprise a target-hybridizing region, e.g., configured to hybridize specifically to a target nucleic acid of an enteric pathogen selected from Yersinia enterocolitica, Vibrio spp. (e.g., V. parahaemolyticus, V. vulnificus, and/or V. cholerae), STEC O157, and Plesiomonas shigelloides. While oligonucleotides of different lengths and base composition may be used for amplifying target nucleic acids, in some embodiments, oligonucleotides in this disclosure have target-hybridizing regions from about 10 to about 60 bases in length, from about 14 to about 50 bases in length, from about 14 to about 40 bases in length, from about 14 to about 35 bases in length, from about 15 to about 30 bases in length, or from about 16 to about 30 bases in length. In some embodiments, an oligonucleotide comprises a second region of sequence in addition to the target-hybridizing region, such as a promoter, which can be located 5′ of the target-hybridizing region. In some embodiments, an oligonucleotide does not comprise a second region of sequence.

In some embodiments, a set of oligonucleotides comprising a combination of two or more oligonucleotides are provided, e.g., in a kit or composition, such as an amplification oligomer (e.g., primer) pair or an amplification oligomer pair and a third oligonucleotide that is optionally labeled (e.g., for use as a probe), wherein the oligonucleotides are configured to hybridize to a target nucleic acid of at least one enteric pathogen selected from Yersinia enterocolitica, Vibrio spp. (e.g., V. parahaemolyticus, V. vulnificus, and/or V. cholerae), STEC O157, and Plesiomonas shigelloides. In some embodiments, the set of oligonucleotides comprises a plurality of amplification oligomer (e.g., primer) pairs or a plurality of amplification oligomer pairs and third oligonucleotides that are optionally labeled (e.g., for use as probes), wherein the oligonucleotides are configured to collectively hybridize to target nucleic acids of at least two enteric pathogens selected from Yersinia enterocolitica, Vibrio spp. (e.g., V. parahaemolyticus, V. vulnificus, and/or V. cholerae), STEC) O157, and Plesiomonas shigelloides.

In some embodiments, one or more oligonucleotides comprise a non-Watson Crick (NWC) position. In some embodiments, a Yersinia enterocolitica amplification oligomer, a Yersinia enterocolitica amplification oligomer pair, and/or a Yersinia enterocolitica probe comprises a NWC position, such as a position that includes inosine. In some embodiments, a Vibro spp. amplification oligomer, a Vibro spp. amplification oligomer pair, and/or a Vibro spp. probe comprises a NWC position, such as a position that includes inosine. In some embodiments, a STEC O157 amplification oligomer, a STEC O157 amplification oligomer pair, and/or a STEC O157 probe comprises a NWC position, such as a position that includes inosine. In some embodiments, a Plesiomonas shigelloides amplification oligomer, a Plesiomonas shigelloides amplification oligomer pair, and/or a Plesiomonas shigelloides probe comprises a NWC position, such as a position that includes inosine.

In some embodiments, one or more oligonucleotides comprise a position comprising 5-methylcytosine. In some embodiments, a Yersinia enterocolitica amplification oligomer, a Yersinia enterocolitica amplification oligomer pair, and/or a Yersinia enterocolitica probe comprises a position comprising 5-methylcytosine. In some embodiments, a Vibro spp. amplification oligomer, a Vibro spp. amplification oligomer pair, and/or a Vibro spp. probe comprises a position comprising 5-methylcytosine. In some embodiments, a STEC O157 amplification oligomer, a STEC O157 amplification oligomer pair, and/or a STEC O157 probe comprises a position comprising 5-methylcytosine. In some embodiments, a Plesiomonas shigelloides amplification oligomer, a Plesiomonas shigelloides amplification oligomer pair, and/or a Plesiomonas shigelloides probe comprises a position comprising 5-methylcytosine.

In some embodiments, one or more oligonucleotides comprise a position comprising propyne dU. In some embodiments, a Yersinia enterocolitica amplification oligomer, a Yersinia enterocolitica amplification oligomer pair, and/or a Yersinia enterocolitica probe comprises a position comprising propyne dU. In some embodiments, a Vibro spp. amplification oligomer, a Vibro spp. amplification oligomer pair, and/or a Vibro spp. probe comprises a position comprising propyne dU. In some embodiments, a STEC O157 amplification oligomer, a STEC O157 amplification oligomer pair, and/or a STEC O157 probe comprises a position comprising propyne dU. In some embodiments, a Plesiomonas shigelloides amplification oligomer, a Plesiomonas shigelloides amplification oligomer pair, and/or a Plesiomonas shigelloides probe comprises a position comprising propyne dU.

Exemplary oligomers targeting Yersinia enterocolitica, Vibrio spp., STEC O157, or Plesiomonas shigelloides target nucleic acid in accordance with the present disclosure are shown in Table 44. Exemplary amplification oligomer pairs and optional third oligomers (e.g., detection probe) are set forth (by SEQ ID NO) in the following Table 1.

TABLE 1
Exemplary Oligonucleotide Sets

| | | | Oligonu- |
|---|---|---|---|
| | Oligonu- | Oligonu- | cleotide 3 |
| | cleotide 1 | cleotide 2 | (e.g., probe, |
| Target pathogen | (e.g., forward | (e.g., reverse | optionally |
| (Target gene) | primer) | primer) | labeled) |
| Y. enterocolitica | 97 | 4 | 50 |
| (ail) | 10 | 4 | 50 |
| | 102 | 24 | 63, 83 |
| | 6 | 24 | 63, 83 |
| Y. enterocolitica | 26 | 46 | 40 |
| (invA) | 13 | 22 | 29 |
| | 77 | 22 | 29 |
| | 77 | 41 | 34 |
| | 28 | 30 | 27 |
| | 60 | 45 | 25 |
| Y. enterocolitica | 65 | 54 | 18 |
| (ystA) | | | |
| V. parahaemolyticus | 51 | 76 | 33, 89, 91 |
| (gyrB) | 58 | 21 | 15 |
| | 58 | 86 | 15 |
| | 53 | 75 | 33, 89, 91 |
| | 58 | 20 | 15 |
| | 52 | 75 | 33, 89, 91 |
| | 51 | 75 | 33, 89, 91 |
| | 52 | 76 | 33, 89, 91 |
| | 53 | 76 | 33, 89, 91 |
| V. vulnificus | 66 | 62 | 9 |
| (gvrB) | 58 | 21 | 31 |
| | 42 | 37 | 9 |
| | 55 | 37 | 9 |
| | 43 | 78 | 31 |
| | 58 | 20 | 31 |
| | 42 | 84 | 9 |
| | 42 | 47 | 9 |
| | 55 | 84 | 9 |
| | 55 | 47 | 9 |
| | 55 | 5 | 9 |
| V. cholerae | 32 | 3 | 35, 88 |
| (ompW) | 85 | 3 | 35, 88 |
| | 92 | 3 | 35, 88 |
| | 92 | 105 | 35, 88 |
| | 32 | 105 | 35, 88 |
| | 85 | 105 | 35, 88 |
| | 56 | 11 | 12 |
| | 57 | 64 | 35, 88 |
| V. cholerae | 94 | 19 | 67, 82 |
| (toxR) | | | |
| STEC O157 | 70 | 16 | 73, 74, 7, 59 |
| (rfbE) | 70 | 44 | 73, 74, 7, 59 |
| | 70 | 107 | 73, 74, 7, 59 |
| | 70 | 87 | 73, 74, 7, 59 |
| | 71 | 16 | 73, 74, 7, 59 |
| | 71 | 44 | 73, 74, 7, 59 |
| | 71 | 107 | 73, 74, 7, 59 |
| | 71 | 87 | 73, 74, 7, 59 |
| | 93 | 101 | 99, 104 |
| | 90 | 100 | 96, 103 |
| P. shigelloides | 2 | 49 | 48 |
| (hugA) | 95 | 1 | 80, 72, 61 |
| | 95 | 106 | 80, 72, 61 |
| | 95 | 98 | 80, 72, 61 |
| | 17 | 1 | 80, 72, 61 |
| | 17 | 106 | 80, 72, 61 |
| | 17 | 98 | 80, 72, 61 |
| | 79 | 81 | 8 |
| | 79 | 68 | 8 |

Exemplary oligomers containing one or more nucleotide analogues (“modified oligomers”) are set forth by SEQ ID NO in the following Table 2 below.

TABLE 2
Exemplary Modified Oligomers

| Modified Oligomer | Corresponds to |
|---|---|
| SEQ ID NO | SEQ ID NO* |
| 108 | 76 |
| 109 | 11 |
| 110 | 66 |
| 111 | 62 |
| 112 | 70 |
| 113 | 16 |
| 114 | 40 |
| 115 | 12 |
| 116 | 73 |
| 117 | 48 |
| 118 | 44 |
| 119 | 74 |
| 120 | 7 |
| 122 | 46 |
| 124 | 29 |
| 125 | 41 |
| 126 | 34 |
| 127 | 28 |
| 128 | 27 |
| 129 | 45 |
| 130 | 25 |
| 131 | 18 |
| 132 | 59 |
| 133 | 22 |
| 134 | 96 |
| 135 | 99 |
| 136 | 106 |

*“Corresponds to” means that the modified oligomer is an example of an oligomer containing one or more nucleotide analogues relative to this SEQ ID NO.

In some embodiments, an oligonucleotide is provided that comprises a label. Such an oligonucleotide can be used as a detection probe. In some embodiments, the labeled oligonucleotide has a sequence corresponding to a SEQ ID NO listed in the Oligonucleotide 3 column of Table 1. In some embodiments, the label is a non-nucleotide label. Suitable labels include compounds that emit a detectable light signal, e.g., fluorophores or luminescent (e.g., chemiluminescent) compounds that can be detected in a homogeneous mixture. More than one label, and more than one type of label, may be present on a particular probe, or detection may rely on using a mixture of probes, in which each probe is labeled with a compound that produces a detectable signal (see. e.g., U.S. Pat. Nos. 6,180,340 and 6,350,579, each incorporated by reference herein). Labels may be attached to a probe by various means including covalent linkages, chelation, and ionic interactions, but in some embodiments the label is covalently attached. For example, in some embodiments, a detection probe has an attached chemiluminescent label such as, e.g., an acridinium ester (AE) compound (see, e.g., U.S. Pat. Nos. 5,185,439; 5,639,604; 5,585,481; and 5,656,744). A label, such as a fluorescent or chemiluminescent label, can be attached to the probe by a non-nucleotide linker (see, e.g., U.S. Pat. Nos. 5,585,481; 5,656,744; and 5,639,604). In some embodiments, the label may include one or more of Quasar670, CalRed610. CalOrange560, fluorescein, ROX, FAM, and HEX.

In some embodiments, a detection probe (e.g., comprising a fluorescent label) further comprises a second label that interacts with the first label. For example, the second label can be a quencher. In some embodiments, the second label may include one or both of BHQ-1 and BHQ-2. Such probes can be used, e.g., in TaqMan™ assays, where hybridization of the probe to a target or amplicon followed by nucleolysis by a polymerase comprising 5′-3′ exonuclease activity results in liberation of the fluorescent label and thereby increased fluorescence, or fluorescence independent of the interaction with the second label.

In some applications, one or more detection probes exhibiting at least some degree of self-complementarity are used to facilitate detection of probe:target duplexes in a test sample without first requiring the removal of unhybridized probe prior to detection. Specific embodiments of such detection probes include, for example, probes that form conformations held by intramolecular hybridization, such as conformations generally referred to as hairpins. Suitable hairpin probes include a “molecular torch” (see, e.g., U.S. Pat. Nos. 6,849,412; 6,835,542; 6,534,274; and 6,361,945) and a “molecular beacon” (see, e.g., U.S. Pat. Nos. 5,118,801 and 5,312,728). Molecular torches include distinct regions of self-complementarity (coined “the target-binding domain” and “the target-closing domain”) which are connected by a joining region (e.g., a —(CH₂CH₂O)₃— linker) and which hybridize to one another under predetermined hybridization assay conditions. When exposed to an appropriate target or denaturing conditions, the two complementary regions (which may be fully or partially complementary) of the molecular torch melt, leaving the target-binding domain available for hybridization to a target sequence when the predetermined hybridization assay conditions are restored. Molecular torches are designed so that the target-binding domain favors hybridization to the target sequence over the target-closing domain. The target-binding domain and the target-closing domain of a molecular torch include interacting labels (e.g., fluorescent/quencher) positioned so that a different signal is produced when the molecular torch is self-hybridized as opposed to when the molecular torch is hybridized to a target nucleic acid, thereby permitting detection of probe:target duplexes in a test sample in the presence of unhybridized probe having a viable label associated therewith.

Examples of interacting donor/acceptor label pairs that may be used in connection with the disclosure, making no attempt to distinguish FRET from non-FRET pairs, include fluorescein/tetramethylrhodamine, IAEDANS/fluorescein, EDANS/DABCYL, coumarin/DABCYL, fluorescein/fluorescein, BODIPY FL/BODIPY FL, fluorescein/DABCYL, lucifer yellow/DABCYL, BODIPY/DABCYL, eosine/DABCYL, erythrosine/DABCYL, tetramethylrhodamine/DABCYL, Texas Red/DABCYL, CY5/BHQ-1, CY5/BHQ-2, CY3/BHQ-1, CY3/BHQ-2 and fluorescein/QSY7 dye. Those having an ordinary level of skill in the art will understand that when donor and acceptor dyes are different, energy transfer can be detected by the appearance of sensitized fluorescence of the acceptor or by quenching of donor fluorescence. Non-fluorescent acceptors such as DABCYL and the QSY7 dyes advantageously eliminate the potential problem of background fluorescence resulting from direct (i.e., non-sensitized) acceptor excitation. Exemplary fluorophore moieties that can be used as one member of a donor-acceptor pair include fluorescein, ROX, and the CY dyes (such as CY5). Exemplary quencher moieties that can be used as another member of a donor-acceptor pair include DABCYL and the Black Hole Quencher moieties which are available from Biosearch Technologies, Inc., (Novato, Calif.).

In some embodiments, a labeled oligonucleotide (e.g., probe) is non-extendable. For example, the labeled oligomer can be rendered non-extendable by 3′-phosphorylation, having a 3′-terminal 3′-deoxynucleotide (e.g., a terminal 2′,3′-dideoxynucleotide), having a 3′-terminal inverted nucleotide (e.g., in which the last nucleotide is inverted such that it is joined to the penultimate nucleotide by a 3′ to 3′ phosphodiester linkage or analog thereof, such as a phosphorothioate), or having an attached fluorophore, quencher, or other label that interferes with extension (possibly but not necessarily attached via the 3′ position of the terminal nucleotide). In some embodiments, the 3′-terminal nucleotide is not methylated.

Also provided by the disclosure is a reaction mixture for determining the presence or absence of a target nucleic acid of at least one enteric pathogen in accordance with the methods as described herein. A reaction mixture in accordance with the present disclosure comprises at least one or more of the following: an oligonucleotide as described herein for amplification of a target nucleic acid; and an oligonucleotide (e.g., probe) as described herein for determining the presence or absence of an amplification product of the target nucleic acid. For a reaction mixture that includes a detection probe together with an amplification oligonucleotide combination, the amplification oligonucleotides and detection probe oligonucleotides for a reaction mixture are linked by a common target region (i.e., the reaction mixture will include a probe that binds to a sequence amplifiable by an amplification oligonucleotides combination of the reaction mixture).

A reaction mixture may further include a number of optional components such as, for example, capture probes, e.g., poly-(k) capture probes as described in US 2013/0209992, which is incorporated herein by reference, and/or poly-(R) capture probes as described in US 2020/0165599, which is incorporated herein by reference. For an amplification reaction mixture, the reaction mixture will typically include other reagents suitable for performing in vitro amplification such as, e.g., buffers, salt solutions, appropriate nucleotide triphosphates (e.g., dATP, dCTP, dGTP, and dTTP; and/or ATP, CTP, GTP and UTP), and/or enzymes (e.g., a thermostable DNA polymerase, or reverse transcriptase and/or RNA polymerase), and will typically include test sample components, in which a target nucleic acid may or may not be present. Suitable reagents include, for example, formulations containing lithium lauryl sulfate (LLS), sodium lauryl sulfate (SLS), NaH₂PO₄, Na₂HPO₄, EDTA, EGTA, LiOH, NaCl, KCl, MgCl, NaOH, ethanol, methylparaben, propylparaben, trehalose. Tris Buffer, Triton X-100, paramagnetic particles, target capture oligonucleotides. HEPES, succinic acid, polymerases (e.g., DNA polymerases, reverse transcriptases), and/or RNasin.

In some embodiments, a reaction mixture comprises KCl. In some embodiments, the KCl concentration is about 50 mM. In some embodiments, the KCl concentration is greater than about 50 mM, e.g., about 60-150 mM, about 75-125 mM, about 80-120 mM, about 85-115 mM, or about 90-110 mM. In some embodiments, the KCl concentration is about 55-65 mM, about 65-75 mM, about 75-85 mM, about 85-95 mM, about 95-105 mM, about 105-115 mM, about 115-125 mM, about 125-135 mM, or about 135-145 mM. In some embodiments, a composition according to the disclosure comprises KCl, e.g., at any of the foregoing concentrations. In some embodiments, a method according to the disclosure comprises performing an amplification reaction in the presence of KCl, e.g., at any of the foregoing concentrations.

In some embodiments, a reaction mixture comprises a non-linear surfactant such as, for example, polysorbate 20. In certain variations, the non-linear surfactant (e.g., polysorbate 20) is present in the reaction mixture at a concentration of from about 0.001% to about 0.025% (v/v) or from about 0.0015% to about 0.015% (v/v). In certain embodiments, a reaction mixture comprises α-cyclodextrin. In certain variations, the α-cyclodextrin is present at a concentration from about 1.0 mg/mL to about 10 mg/mL, from about 3.0 mg/mL to about 9.0 mg/mL, or from about 2.0 mg/mL to about 7.0 mg/mL.

In some embodiments, a reaction mixture comprises a lyoprotectant. Exemplary lyoprotectants include glycerol: non-reducing sugars such as, e.g., sucrose, raffinose, or trehalose; and amino acids such as, e.g., glycine, arginine, or methionine. In certain variations wherein the lyoprotectant is trehalose, trehalose is present at a concentration of from about 0.1 M to about 0.2 M (e.g., about 0.15 M).

In some embodiments, a reaction mixture comprises a chelating agent. Suitable chelating agents include ethylenediaminetetraacetic acid (EDTA) and ethylene glycol-bis(β-aminoethyl ether)-N,N,N′,N′-tetraacetic acid (EGTA). In some embodiments comprising EDTA as the chelating agent, EDTA is present in the reaction mixture at a concentration of from about 0.025 mM to about 0.25 mM (e.g., at a concentration of about 0.08 mM).

Also provided by the subject disclosure are kits for practicing the methods as described herein. A kit in accordance with the present disclosure comprises at least one or more of the following: an oligonucleotide as described herein for amplification of a target nucleic acid; and an oligonucleotide (e.g., probe) as described herein for determining the presence or absence of an amplification product of the target nucleic acid. In some embodiments, any oligonucleotide combination described herein is present in the kit. The kits may further include a number of optional components such as, for example, capture probes, e.g., poly-(k) capture probes as described in US 2013/0209992 and/or poly-(R) capture probes as described in US 2020/0165599. Other reagents that may be present in the kits include reagents suitable for performing in vitro amplification such as, e.g., buffers, salt solutions, appropriate nucleotide triphosphates (e.g., dATP, dCTP, dGTP, dTTP; and/or ATP. CTP, GTP and UTP), and/or enzymes (e.g., a thermostable DNA polymerase, or a reverse transcriptase and/or RNA polymerase). Oligonucleotides as described herein may be packaged in a variety of different embodiments, and those skilled in the art will appreciate that the disclosure embraces many different kit configurations. For example, a kit may include amplification oligonucleotides for only one, two, three, or all of Yersinia enterocolitica, Vibrio spp., STEC O157, and Plesiomonas shigelloides. In addition, for a kit that includes a detection probe together with an amplification oligomer combination, the amplification oligonucleotides and detection probe oligonucleotides for a reaction mixture are linked by a common target region (i.e., the reaction mixture will include a probe that binds to a sequence amplifiable by an amplification oligonucleotides combination of the reaction mixture). In certain embodiments, the kit further includes a set of instructions for practicing methods in accordance with the present disclosure, where the instructions may be associated with a package insert and/or the packaging of the kit or the components thereof.

In some embodiments of a kit as described herein, the oligonucleotides are contained in a formulation comprising at least one of a non-linear surfactant (e.g., polysorbate 20), α-cyclodextrin, a lyoprotectant (e.g., a non-reducing sugar such as sucrose, raffinose, or trehalose, or an amino acid such as glycine, arginine, or methionine), and a chelating agent (e.g., EDTA or EGTA). In some such embodiments, polysorbate 20 is present in the formulation at a concentration of from about 0.002% to about 0.05% (v/v) or about 0.003% to about 0.03% (v/v), α-cyclodextrin is present at a concentration from about 1.0 mg/mL to about 10 mg/mL or about 3.0 mg/mL to about 9 mg/mL, trehalose is present in the formulation at a concentration of from about 0.2 M to about 0.4 M (e.g., about 0.26 M or about 0.3 M), and/or EDTA is present in the formulation at a concentration of from about 0.05 mM to about 0.5 mM (e.g., about 0.16 mM or about 0.14 mM). In certain variations, the formulation is a lyophilized formulation. In some embodiments of a lyophilized formulation, the formulation is for reconstitution into an aqueous formulation containing polysorbate 20, trehalose, and/or EDTA at concentration(s) as specified above.

Also provided by the subject disclosure are methods (e.g., multiplex methods) for determining the presence or absence of at least one enteric pathogen, including Yersinia enterocolitica. Vibrio spp., STEC O157, and/or Plesiomonas shigelloides, in a sample by, for example, using one or more of the oligonucleotides disclosed herein. Any method disclosed herein is also to be understood as a disclosure of corresponding uses of materials involved in the method directed to the purpose of the method. Any of the oligonucleotides and any combinations (e.g., kits and compositions) comprising such an oligonucleotide are to be understood as also disclosed for use in detecting enteric pathogen target nucleic acid and for use in the preparation of a composition for detecting enteric pathogen target nucleic acid.

Broadly speaking, methods can comprise one or more of the following components: target capture, in which a target nucleic acid (e.g., from a sample, such as a clinical sample) is annealed to a capture oligomer: isolation, e.g., washing, to remove material not associated with a capture oligomer; amplification; and amplicon detection, e.g., amplicon quantification, which may be performed in real time with amplification. Certain embodiments involve each of the foregoing steps. Certain embodiments involve exponential amplification, optionally with a preceding linear amplification step. Certain embodiments involve exponential amplification and amplicon detection. Certain embodiments involve any two of the components listed above. Certain embodiments involve any two components listed adjacently above, e.g., washing and amplification, or amplification and detection.

Amplifying an enteric pathogen target nucleic acid region utilizes an in vitro amplification reaction using at least two amplification oligomers that flank a target region to be amplified (e.g., one or more oriented in the sense direction and one or more oriented in the antisense direction for exponential amplification). Particularly suitable oligomer combinations for amplification of Yersinia enterocolitica. Vibrio spp., STEC O157, and/or Plesiomonas shigelloides target regions are described herein. Exemplary amplification oligomers for amplifying enteric pathogen target regions are listed in Table 44, infra (see also exemplary modified oligomers in Table 2, supra), and particular combinations of first and second amplification oligomers for each of Yersinia enterocolitica, Vibrio parahaemolyticus, Vibrio vulnificus, Vibrio cholerae, STEC O157, and/or Plesiomonas shigelloides are set forth herein (see, e.g., Embodiments section and Table 1, supra, and Examples 2-14, infra (including Tables 3, 5, 8, 10, 12, 13, 15, 18, 22, 25, 28, 32, 33, and 38).

A detection method in accordance with the present disclosure can further include the step of obtaining the sample to be subjected to subsequent steps of the method. In certain embodiments, “obtaining” a sample to be used includes, for example, receiving the sample at a testing facility or other location where one or more steps of the method are performed, and/or retrieving the sample from a location (e.g., from storage or other depository) within a facility where one or more steps of the method are performed.

In certain embodiments, the method further includes purifying the enteric pathogen target nucleic acid from other components in the sample, e.g., before an amplification, such as before a capture step. Such purification may include methods of separating and/or concentrating organisms contained in a sample from other sample components, or removing or degrading non-nucleic acid sample components, e.g., protein, carbohydrate, salt, lipid, etc. In some embodiments, purifying the target nucleic acid includes degrading nucleic acid in the sample, e.g., with DNase, and optionally removing or inactivating the DNase or removing degraded nucleic acid.

In particular embodiments comprising a target purification step, a target nucleic acid is captured specifically or non-specifically and separated from other sample components. Non-specific target capture methods may involve selective precipitation of nucleic acids from a substantially aqueous mixture, adherence of nucleic acids to a support that is washed to remove other sample components, or other means of physically separating nucleic acids from a mixture that contains enteric pathogen nucleic acid and other sample components.

Target capture typically occurs in a solution phase mixture that contains one or more capture probe oligomers that hybridize to the enteric pathogen target nucleic acid under hybridizing conditions. For embodiments comprising a capture probe tail, the target:capture-probe complex is captured by adjusting the hybridization conditions so that the capture probe tail hybridizes to an immobilized probe. Certain embodiments use a particulate solid support, such as paramagnetic beads. Selective and non-specific target capture methods are also described, e.g., in U.S. Pat. No. 6,110,678 and International Patent Application Pub. No. WO 2008/016988, each incorporated by reference herein.

Isolation can follow capture, where, for example, the complex on the solid support is separated from other sample components. Isolation can be accomplished by any appropriate technique, e.g., washing a support associated with the enteric pathogen target nucleic acid one or more times (e.g., two or three times) to remove other sample components and/or unbound oligomer. In embodiments using a particulate solid support, such as paramagnetic beads, particles associated with the enteric pathogen target may be suspended in a washing solution and retrieved from the washing solution, in some embodiments by using magnetic attraction. To limit the number of handling steps, the enteric pathogen target nucleic acid may be amplified by simply mixing the target region in the complex on the support with amplification oligomers and proceeding with amplification steps.

Exponentially amplifying a target sequence utilizes an in vitro amplification reaction using at least two amplification oligomers that flank a target region to be amplified. In some embodiments, at least one oligonucleotide as described above is provided. In some embodiments, a plurality of pairs of oligonucleotides is provided, wherein the plurality comprises oligonucleotides pairs configured to hybridize to at least one, two, three, or all of Yersinia enterocolitica, Vibrio spp., STEC O157, and Plesiomonas shigelloides target nucleic acids. The amplification reaction can be cycled or isothermal. Suitable amplification methods include, for example, replicase-mediated amplification, polymerase chain reaction (PCR), ligase chain reaction (LCR), strand-displacement amplification (SDA), and transcription-mediated or transcription-associated amplification (TMA).

A detection step may be performed using any of a variety of known techniques to detect a signal specifically associated with the amplified target region, such as, e.g., by hybridizing the amplification product with a labeled detection probe and detecting a signal resulting from the labeled probe (including from label released from the probe following hybridization in some embodiments), performing electrophoresis on the sample and/or the amplification product, or determining the sequence of the amplification product. In some embodiments, the labeled probe comprises a second moiety, such as a quencher or other moiety that interacts with the first label, as discussed above. The detection step may also provide additional information on the amplified sequence, such as, e.g., all or a portion of its nucleic acid base sequence. Detection may be performed after the amplification reaction is completed or may be performed simultaneously with amplifying the target region, e.g., in real time. In one embodiment, the detection step allows homogeneous detection, e.g., detection of the hybridized probe without removal of unhybridized probe from the mixture (see, e.g., U.S. Pat. Nos. 5,639,604 and 5,283,174). In some embodiments, the nucleic acids are associated with a surface that results in a physical change, such as a detectable electrical change. Amplified nucleic acids may be detected by concentrating them in or on a matrix and detecting the nucleic acids or dyes associated with them (e.g., an intercalating agent such as ethidium bromide or cyber green) or detecting an increase in dye associated with nucleic acid in solution phase. Other methods of detection may use nucleic acid detection probes that are configured to specifically hybridize to a sequence in the amplified product and detecting the presence of the probe:product complex, or by using a complex of probes that may amplify the detectable signal associated with the amplified products (see, e.g., U.S. Pat. Nos. 5,424,413; 5,451,503; and 5,849,481; each incorporated by reference herein). Directly or indirectly labeled probes that specifically associate with the amplified product provide a detectable signal that indicates the presence of the target nucleic acid in the sample. In particular, the amplified product will contain a target sequence in or complementary to a target nucleic sequence of at least one enteric pathogen, and a probe will bind directly or indirectly to a sequence contained in the amplified product to indicate the presence or absence of the pathogen in the tested sample.

In embodiments that detect the amplified product near or at the end of the amplification step, a linear detection probe may be used to provide a signal to indicate hybridization of the probe to the amplified product. One example of such detection uses a luminescently labeled probe that hybridizes to target nucleic acid. The luminescent label is then hydrolyzed from non-hybridized probe. Detection is performed by chemiluminescence using a luminometer. (See, e.g., International Patent Application Pub. No. WO 89/002476, incorporated by reference herein). In other embodiments that use real-time detection, the detection probe may be a hairpin probe such as, for example, a molecular beacon, molecular torch, or hybridization switch probe that is labeled with a reporter moiety that is detected when the probe binds to amplified product (e.g., a dual-labeled hairpin probe comprising both a fluorescent label and a quenching moiety). In other embodiments for real-time detection, the detection probe is a linear oligomer such as, e.g., an oligomer labeled with both a fluorophore and a quenching moiety (e.g., a TaqMan probe). Such probes may comprise target-hybridizing sequences and non-target-hybridizing sequences. Various forms of such probes have been described previously (see, e.g., U.S. Pat. Nos. 5,210,015; 5,487,972; 5,118,801; 5,312,728; 5,925,517; 6,150,097; 6,849,412; 6,835,542; 6,534,274; and 6,361,945; and US Patent Application Pub. Nos. 20060068417A1 and 20060194240A1: each incorporated by reference herein). Exemplary enteric-pathogen-specific detection probe oligomers are listed in Tables 1 and 2, supra, and Table 44, infra, and are also set forth in the Embodiments section, supra, and Examples, infra (including. e.g., their use in combination with at least two enteric-pathogen-specific amplification oligomers for detection of an enteric pathogen target nucleic acid).

Assays for detection of an enteric pathogen nucleic acid may optionally include a non-enteric-pathogen internal control (IC) nucleic acid that is amplified and detected in the same assay reaction mixtures by using amplification and detection oligomers specific for the IC sequence. IC nucleic acid sequences can be, e.g., a DNA plasmid, an RNA template sequence (e.g., an in vitro transcript), or a synthetic nucleic acid that is spiked into a sample. Alternatively, the IC nucleic acid sequence may be a cellular component, which may be from exogenous cellular sources or endogenous cellular sources relative to the specimen. In these instances, an internal control nucleic acid is co-amplified with the enteric pathogen nucleic acid in the amplification reaction mixtures. The internal control amplification product and the enteric pathogen target region amplification product can be detected independently.

In certain embodiments, amplification and detection of a signal from an amplified IC sequence demonstrates that the assay reagents, conditions, and performance of assay steps were properly used in the assay if no signal is obtained for an intended target enteric pathogen nucleic acid (e.g., samples that test negative for the enteric pathogen). An IC may also be used as an internal calibrator for the assay when a quantitative result is desired, i.e., the signal obtained from the IC amplification and detection is used to set a parameter used in an algorithm for quantitating the amount of enteric pathogen nucleic acid in a sample based on the signal obtained for an amplified enteric pathogen target region. ICs are also useful for monitoring the integrity of one or more steps in an assay. The primers and probe for the IC target sequence are configured and synthesized by using any well-known method provided that the primers and probe function for amplification of the IC target sequence and detection of the amplified IC sequence using substantially the same assay conditions used to amplify and detect the enteric pathogen target region(s). In certain embodiments that include a target capture-based purification step, it is preferred that a target capture probe specific for the IC target be included in the assay in the target capture step so that the IC is treated in the assay in a manner analogous to that for the intended enteric pathogen analyte(s) in all of the assay steps.

Methods (e.g., multiplex methods) for determining the presence or absence of at least one enteric pathogen as described herein may have a detection sensitivity from 10 to 500 CFU/mL, from 25 to 500 CFU/mL, from 50 to 500 CFU/mL, from 75 to 500 CFU/mL, from 100 to 500 CFU/mL, from 10 to 300 CFU/mL, from 25 to 300 CFU/mL, from 50 to 300 CFU/mL, from 75 to 300 CFU/mL, from 100 to 300 CFU/mL, from 10 to 150 CFU/mL, from 25 to 150 CFU/mL, from 50 to 150 CFU/mL, from 75 to 150 CFU/mL, from 100 to 150 CFU/mL, from 10 to 100 CFU/mL, from 25 to 100 CFU/mL, from 50 to 100 CFU/mL, from 75 to 100 CFU/mL, from 10 to 75 CFU/mL, from 25 to 75 CFU/mL, from 50 to 75 CFU/mL, from 10 to 50 CFU/mL, or from 25 to 50 CFU/mL.

Also provided by the subject disclosure are methods for synthesizing one or more (e.g., one or more pairs) of the oligonucleotides disclosed herein, the oligonucleotides useful for determining the presence or absence of at least one enteric pathogen selected from Yersinia enterocolitica, Vibrio spp. (e.g., V. parahaemolyticus, V. vulnificus, and/or V. cholerae), STEC O157, and Plesiomonas shigelloides. The method may, for example, include the steps of (a) obtaining a solid support comprising at least one nucleobase residue, wherein the at least one nucleobase residue is bound (e.g., covalently bound) at a 3′ position to the solid support; (b) coupling a 5′ position of the nucleobase residue furthest from the solid support to a 3′ position of another nucleobase residue; (c) repeating step (b) at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, or at least 28 additional times, thereby generating at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleobase residues coupled to the solid support; and (d) cleaving the at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleobase residues generated in step (c), thereby obtaining the oligonucleotide or oligonucleotides. In some embodiments, the oligonucleotide has a length of from 18 to 32 contiguous nucleobase residues. In some embodiments, the oligonucleotide has a length of from 20 to 30 contiguous nucleobase residues.

A method for synthesizing one or more of the oligonucleotides disclosed herein may be a solid phase method. For example, phosphoramidite solid-phase chemistry for joining nucleotides by phosphodiester linkages is disclosed in Caruthers et al., “Chemical Synthesis of Deoxynucleotides by the Phosphoramidite Method,” Methods Enzymol. 154:287 (1987). As another example, automated solid-phase chemical synthesis using cyanoethyl phosphoramidite precursors has been described in Barone et al., “In Situ Activation of bis-dialkylaminephosphines—a New Method for Synthesizing Deoxyoligonucleotides on Polymer Supports,” Nucleic Acids Res. 12(10):4051 (1984). As another example. U.S. Pat. No. 5,449,769, titled “Method and Reagent for Sulfurization of Organophosphorous Compounds.” discloses a procedure for synthesizing oligonucleotides containing phosphorothioate linkages. In addition. U.S. Pat. No. 5,811,538, titled “Process for the Purification of Oligomers,” discloses the synthesis of oligonucleotides having different linkages, including methylphosphonate linkages. Moreover, methods for the organic synthesis of oligonucleotides are described in, for example, Sambrook et al., Molecular Cloning, A Laboratory Manual, 2nd ed. (Cold Spring Harbor Laboratory Press, Cold Spring Harbor, NY, 1989) at Chapt. 10.

Following synthesis and purification of a particular oligonucleotide, several different procedures may be utilized to purify and control the quality of the oligonucleotide. Suitable procedures include electrophoresis (e.g., polyacrylamide gel electrophoresis) or chromatography (e.g., high pressure liquid chromatography).

The compositions, kits, formulations, reaction mixtures, and methods are further illustrated by the following non-limiting examples.

### EXAMPLES

### Example 1: Real-Time PCR Amplification and Detection of GI-Bacterial Panel Targets Using Different Combinations of Primers and Probes

Several primer and probe combinations for real-time PCR amplification and detection of Yersinia enterocolitica, Vibrio spp, Shiga Toxin Expressing E. coli (STEC O157), and Plesiomonas shigelloides targets were tested.

Amplification and detection reactions were performed using a Panther Fusion instrument (Hologic. Inc. San Diego, CA). Typically, 20 μL of an amplification reagent was combined in a reaction well of a multi-well plate with 5 μL of a target nucleic acid. The multi-well plate was placed in the Panther Fusion instrument and subjected to thermal cycling. Real-time amplification and detection reactions were performed by thermal cycling, generally for 45 cycles (denaturation at 95° C. for 8 seconds and annealing and extension at 60° C. for 25 seconds), taking fluorescent emission readings every 30 seconds. Fluorescence curve profiles for the target nucleic acids were evaluated for Ct and RFU signals. The assay targeted DNA and did not include a reverse transcriptase (RT) extension step.

### Example 2: Screening of Primers and Probes for Amplification and Detection of Vibrio cholera

Two primer and probe sets for Vibrio cholerae detection were tested in multiplex with a primer and probe set for detection of Vibrio parahaemolyticus and Vibrio vulnificus. V cholerae panels were tested at 1e4 CFU/mL and 1e3 CFU/mL, and the V. parahaemolyticus panel was tested at 1e2 CFU/mL, 1e3 CFU/mL, and 1e4 CFU/mL. The primers and probes used in the experiment are shown in Table 3.

TABLE 3

| | | SEQ |
|---|---|---|
| Oligo Set | Oligo Type | ID NO |
| Vibrio | Primer | 58 |
| parahaemolyticus | Primer | 21 |
| | Probe | 15 |
| | Probe | 31 |
| Vibrio cholerae | Primer | 32 |
| Set 1 | Primer | 3 |
| | Probe | 35 |
| Vibrio cholera | Primer | 57 |
| Set 2 | Primer | 64 |
| | Probe | 35 |
| Internal Control | Primer | 23 |
| (IC) | Primer | 38 |
| | Probe | 36 |

Results are shown in Table 4 below.

TABLE 4

| | | Target | HEX | | RED677 (IC) | |
|---|---|---|---|---|---|---|
| | | conc | Avg | Avg | Avg | Avg |
| Oligo Sets | Target | (CFU/mL) | Ct | RFU | Ct | RFU |
| V. cholerae | V chol | 1E3 | 30.51 | 28020 | 28.18 | 15833 |
| Set 1 | V chol | 1E4 | 28.22 | 27687 | 28.09 | 14194 |
| V. | V para | 1E2 | 35.18 | 10244 | 28.88 | 13104 |
| parahaemolyticus | V para | 1E3 | 30.65 | 16926 | 28.31 | 14655 |
| Set 1 | V para | 1E4 | 30.24 | 17939 | 28.95 | 12557 |
| | Neg | Neg Cntrl | — | 815 | 28.11 | 14016 |
| | Cntrl | | | | | |
| V. cholerae | V chol | 1E3 | 31.24 | 27805 | 28.06 | 15074 |
| Set 2 | V chol | 1E4 | 27.34 | 28327 | 28.21 | 11666 |
| V. | V para | 1E2 | 35.70 | 12903 | 28.72 | 14811 |
| parahaemolyticus | V para | 1E3 | 32.83 | 18677 | 28.64 | 13705 |
| Set 1 | V para | 1E4 | 28.57 | 22343 | 28.76 | 13967 |
| | Neg | — | 34.88 | 1488 | 28.12 | 14355 |
| | Cntrl | | | | | |

Vibrio cholerae Set 1 and Set 2 amplified well in the 1e4 and 1e3 CFU/mL panels. The baseline rose in these amplification curves so the analysis parameters may need to be adjusted for the selected design set.

Vibrio parahaemolyticus Set 1 amplified well in the presence of both V. cholerae Set 1 and Set 2. 1e2 CFU/mL was able to be detected with both systems making this a potential selected system for V. parahaemolyticus. The same analysis parameters would be adjusted for these amplification curves as well.

### Example 3: Amplification and Detection of V. Parahaemolyticus and V. Vulnificus Targets Using Dual and Single Amplifications Systems

A primer and probe set for dual detection of Vibrio parahaemolyticus and Vibrio vulnificus (Dual Detection Set) were compared against a combination of individual detection systems for V. parahaemolyticus and V. vulnificus (Single Detection Sets). Vibrio parahaemolyticus and Vibrio vulnificus were tested at 1e4 CFU/mL and 1e3 CFU/mL. The primers and probes used in the experiment are shown in Table 5.

TABLE 5

| | Oligo | SEQ |
|---|---|---|
| Oligo Set | Type | ID NO |
| Dual Detection Set | Primer | 58 |
| | Primer | 21 |
| | Probe | 15 |
| | Probe | 31 |
| V. parahaemolyticus | Primer | 53 |
| Single Detection Set | Primer | 75 |
| | Probe | 33 |
| V. vulnificus | Primer | 55 |
| Single Detection Set | Primer | 5 |
| | Probe | 9 |
| Internal Control | Primer | 23 |
| (IC) | Primer | 38 |
| | Probe | 36 |

Results are shown in Tables 6 and 7 below.

TABLE 6
Results for detection of V. parahaemolyticus

| | Target Conc | HEX | | Cy5.5 (IC) | |
|---|---|---|---|---|---|
| System | (CFU/mL) | Ct | RFU | Ct | RFU |
| Dual Detection System | 1E3 | 32.1 | 20234 | 28.2 | 15597 |
| | | 31.3 | 23158 | 28.2 | 17136 |
| | | 31.5 | 21481 | 28.2 | 15102 |
| | 1E4 | 29 | 23464 | 28.1 | 15387 |
| | | 29 | 23183 | 28 | 14731 |
| | | 29.1 | 23604 | 28.3 | 14989 |
| V. parahaemolyticus | 1E3 | 31.3 | 19288 | 28.2 | 11776 |
| Single Detection Set | | 31.1 | 20653 | 28.1 | 12402 |
| | | 31.2 | 20476 | 28 | 13264 |
| | 1E4 | 28.6 | 24120 | 28.4 | 10048 |
| | | 28.4 | 21567 | 28.5 | 9419 |
| | | 28.4 | 23977 | 28.4 | 10273 |

TABLE 7
Results for detection of V. vulnificus

| | Target Conc | HEX | | Cy5.5 (IC) | |
|---|---|---|---|---|---|
| System | (CFU/mL) | Ct | RFU | Ct | RFU |
| Dual Detection | 1E3 | 35.7 | 1367 | 28.1 | 17586 |
| System | | 43.7 | 1039 | 28.2 | 17446 |
| | | — | 778 | 28.2 | 14934 |
| | 1E4 | 40.1 | 2359 | 28.2 | 16012 |
| | | 37.7 | 4194 | 28.4 | 15736 |
| | | 41.7 | 1090 | 28.1 | 18564 |
| V. vulnificus | 1E3 | — | 623 | 28.6 | 14281 |
| Single Detection Set | | — | 652 | 28.6 | 15028 |
| | | — | 569 | 28.6 | 14296 |
| | 1E4 | — | 840 | 29 | 13949 |
| | | — | 811 | 28.7 | 14502 |
| | | — | 741 | 28.8 | 16867 |

Both Vibrio parahaemolyticus Dual and Single detection systems amplified well with detection of both panels at 1e4 and 1e3 CFU/mL. A 0.5-1 Ct earlier Ct is seen for the in the Single Detection system which is a slight improvement over the Dual Detection system.

Both Vibrio vulnificus Dual and Single detection systems showed no amplification in either concentration.

### Example 4: Amplification and Detection of V. Parahaemolyticus, V. Cholerae, and STEC O157 Targets in Multiplex Systems

Two Vibrio cholerae primer and probe sets were tested in multiplex with primer and probe sets for detection of Vibrio parahaemolyticus and STEC O157. All targets were tested as lysate panels in STM (Sample Transport Medium; 3% (w/v) lithium lauryl sulfate (LLS), 0.2% (w/v) NaH₂PO₄, 0.2% (w/v) Na₂HPO₄, 0.04% (w/v) EDTA, 0.04% EGTA (w/v), pH 6.7) at 1e4, 1e3, 1e2, and 1e1 CFU/mL. The primers and probes used in the experiment are shown in Table 8. Both multiplex sets included an internal control (IC) primer and probe set (primers of SEQ ID NOs:23 and 38 and probe of SEQ ID NO:36).

TABLE 8

| Multiplex | | Oligo | SEQ ID | |
|---|---|---|---|---|
| Set | Target | Type | NO | Modifications |
| Set 1 | V. parahaemolyticus | Primer | 58 | |
| | | Primer | 21 | |
| | | Probe | 15 | HEX/BHQ1 |
| | | Probe | 31 | HEX/BHQ1 |
| | V. cholerae | Primer | 32 | |
| | | Primer | 3 | |
| | | Probe | 35 | HEX/BHQ1 |
| | STEC O157 | Primer | 71 | |
| | | Primer | 44 | 5 mC at residues 2, 15, 16, and 24 |
| | | | | pdU at residue 20 |
| | | Probe | 74 | 5 mC at residues 3, 6, 13, 14, 19, and 22 |
| | | | | pdU at residues 4, 9, 17, and 20 |
| | | | | CalRed610/BHQ2 |
| | | Probe | 7 | 5 mC at residues 2, 5, 6, and 20 |
| | | | | pdU at residues 4, 8, 10, 13, 15, and 24 |
| | | | | CalRed610/BHQ2 |
| Set 2 | V. parahaemolyticus | Primer | 58 | |
| | | Primer | 21 | |
| | | Probe | 15 | HEX/BHQ1 |
| | | Probe | 31 | HEX/BHQ1 |
| | V. cholerae | Primer | 57 | |
| | | Primer | 64 | |
| | | Probe | 35 | HEX/BHQ1 |
| | STEC O157 | Primer | 71 | |
| | | Primer | 44 | 5 mC at residues 2, 15, 16, and 24 |
| | | | | pdU at residue 20 |
| | | Probe | 74 | 5 mC at residues 3, 6, 13, 14, 19, and 22 |
| | | | | pdU at residues 4, 9, 17, and 20 |
| | | | | CalRed610/BHQ2 |
| | | Probe | 7 | 5 mC at residues 2, 5, 6, and 20 |
| | | | | pdU at residues 4, 8, 10, 13, 15, and 24 |
| | | | | CalRed610/BHQ2 |

Results are shown in Table 9 below.

TABLE 9

| | | Set 1 | | | Set 2 | | |
|---|---|---|---|---|---|---|---|
| | Target Conc | n pos | Avg | Avg | n pos | Avg | Avg |
| Target | (CFU/mL) | out of 3 | Ct | RFU | out of 3 | Ct | RFU |
| V. cholera | 1E1 | 2 | 34.60 | 7554 | 1 | 37.13 | 4739 |
| | 1E2 | 3 | 30.49 | 19926 | 3 | 33.86 | 19232 |
| | 1E3 | 3 | 31.25 | 24944 | 3 | 31.86 | 23162 |
| | 1E4 | 3 | 28.92 | 29039 | 3 | 29.09 | 27100 |
| V. parahaemolyticus | 1E1 | 1 | 40.09 | 3239 | 1 | 39.57 | 4026 |
| | 1E2 | 3 | 35.64 | 11870 | 3 | 35.09 | 13303 |
| | 1E3 | 3 | 29.82 | 18642 | 3 | 32.83 | 18609 |
| | 1E4 | 3 | 29.64 | 21637 | 3 | 30.31 | 20422 |
| STEC O157 | 1E1 | 2 | 39.01 | 2312 | 3 | 38.93 | 3981 |
| | 1E2 | 3 | 35.79 | 6984 | 3 | 36.24 | 7164 |
| | 1E3 | 3 | 32.81 | 11895 | 3 | 32.97 | 10859 |
| | 1E4 | 3 | 29.22 | 13602 | 3 | 29.37 | 15248 |
| IC (Negative STM) | — | 3 | 28.43 | 15508 | 3 | 28.00 | 18752 |

The STEC O157 oligos perform better with V. cholerae Set 2 system versus Set 1. V. cholerae Set 1 had 2/3 reps positive versus Set 2 that had 1/3 positive. More replicates near LoD will need to be tested to see if this sensitivity is better with Set 1 versus Set 2. V. parahaemolyticus oligos performed similarly with both V. cholerae sets. GIC performed well with both Primer Probe Reagent (PPR) sets.

### Example 5: Amplification and Detection of Yersinia enterocolitica. Vibrio Spp., STEC O157. And Plesiomonas Shigelloides Targets in Multiplex System

Primer and probe sets for detection of Yersinia enterocolitica, Vibrio spp., STEC O157, and Plesiomonas shigelloides were tested in multiplex. All targets were tested as lysate panels in STM at 1e4, 1e3, and 1e2 CFU/mL at two replicates each. STEC O157 was also tested at 1e1 CFU/mL. The primers and probes used in the experiment are shown in Table 10. The multiplex set included an internal control (IC) primer and probe set (primers of SEQ ID NOs:23 and 38 and probe of SEQ ID NO:36).

TABLE 10

| | Oligo | SEQ ID | |
|---|---|---|---|
| Target | Type | NO | Modifications |
| V. parahaemolyticus | Primer | 58 | |
| | Primer | 21 | |
| | Probe | 15 | HEX/BHQ1 |
| | Probe | 31 | HEX/BHQ1 |
| V. cholerae | Primer | 32 | |
| | Primer | 3 | |
| | Probe | 35 | HEX/BHQ1 |
| STEC O157 | Primer | 71 | |
| | Primer | 44 | 5 mC at residues 2, 15, 16, and 24 |
| | | | pdU at residue 20 |
| | Probe | 74 | 5 mC at residues 3, 6, 13, 14, 19, and 22 |
| | | | pdU at residues 4, 9, 17, and 20 |
| | | | CalRed610/BHQ2 |
| | Probe | 7 | 5 mC at residues 2, 5, 6, and 20 |
| | | | pdU at residues 4, 8, 10, 13, 15, and 24 |
| | | | CalRed610/BHQ2 |
| Y. enterocolitica | Primer | 6 | |
| | Primer | 24 | |
| | Probe | 63 | FAM/BHQ1 |
| | | | Reverse polarity C |
| P. shigelloides | Primer | 17 | |
| | Primer | 1 | |
| | Probe | 72 | Quasar670/BHQ2 |

Results are shown in Table 11 below.

TABLE 11

| | Target | n pos | FAM | | Q705 - IC | |
|---|---|---|---|---|---|---|
| | Conc | out | Avg | Avg | Avg | Avg |
| Target | (CFU/mL) | of 2 | Ct | RFU | Ct | RFU |
| Y. enterocolitica | 1E2 | 2 | 34.75 | 29337 | 28.41 | 16059 |
| | 1E3 | 2 | 31.16 | 35065 | 28.08 | 14614 |
| | 1E4 | 2 | 27.51 | 37473 | 28.04 | 12829 |
| | Target | n pos | HEX | | Q705 - IC | |
| | Conc | out | Avg | Avg | Avg | Avg |
| Target | (CFU/mL) | of 3 | Ct | RFU | Ct | RFU |
| V. cholerae | 1E2 | 2 | 36.50 | 17100 | 28.40 | 17201 |
| | 1E3 | 2 | 33.99 | 23597 | 28.49 | 16960 |
| | 1E4 | 2 | 29.95 | 27269 | 27.98 | 15075 |
| V. parahaemolyticus | 1E2 | 2 | 39.17 | 6031 | 28.07 | 17435 |
| | 1E3 | 2 | 34.58 | 17717 | 27.90 | 17858 |
| | 1E4 | 2 | 31.59 | 21543 | 28.22 | 15769 |

| | | | ROX | | Q705 - IC | |
|---|---|---|---|---|---|---|
| | Target Conc | n pos | Avg | Avg | Avg | Avg |
| Target | (CFU/mL) | out of 3 | Ct | RFU | Ct | RFU |
| STEC O157 | 1E1 | 1 | 19.52 | 1794 | 28.25 | 16253 |
| | 1E2 | 2 | 36.83 | 5356 | 28.49 | 16035 |
| | 1E3 | 2 | 32.59 | 10224 | 28.31 | 13952 |
| | 1E4 | 2 | 29.19 | 12532 | 27.95 | 11872 |

| | | | Q670 | | Q705 - IC | |
|---|---|---|---|---|---|---|
| | Target Conc | n pos | Avg | Avg | Avg | Avg |
| Target | (CFU/mL) | out of 3 | Ct | RFU | Ct | RFU |
| P. shigelloides | 1E2 | 2 | 38.12 | 2144 | 27.77 | 17171 |
| | 1E3 | 2 | 34.76 | 2788 | 27.89 | 17207 |
| | 1E4 | 2 | 32.32 | 2876 | 28.50 | 14465 |
| Negative STM | — | 2 | 0.00 | 122 | 28.00 | 18318 |

The multiplex amplified all targets and channels were differentiated well. All Assay Targets were positive at 100 CFU/mL; further sensitivity needs to be established for each panel.

### Example 6: Amplification and Detection of Vibrio Spp. Using Dual and Individual Amplification Systems for V. Vulnificus and V. Parahaemolyticus

A V. vulinficus/V. parahaemolyticus dual amplification system contained a 3′ mismatch for the Vibrio vulnificus design set, which caused the V. vulnificus system to not amplify in the V. vulnificus V. parahaemolyticus dual amplification system. The reverse primer system was modified to remove the mismatch to restore amplification to that target. This modified system was tested and compared to individual V. vulnificus systems to compare amplification performance. The following primer/probe sets were tested to compare performance:
PPR 1: Mismatch Reverse Primer control—Dual system
PPR 2: Modified Reverse Primer—Dual system
PPR 3: V. vulnificus individual assay Set 1
PPR 4: V. vulnificus individual assay Set 2
PPR 5: V. vulnificus individual assay Set 3

All targets were tested as lysate panels in STM at 1e4, 1e3, and 1e2 CFU/mL at three replicates each. The primers and probes used in the experiment are shown in Tables 12 and 13. Each set included an internal control (IC) primer and probe set (primers of SEQ ID NOs:23 and 38 and probe of SEQ ID NO:36). Vibrio spp. probes were labeled with HEX and BHQL, and the IC probe was labeled with Quasar 705 and BHQ2.

TABLE 12
Dual System Oligos

| | Oligo | PPR1 | PPR2 |
|---|---|---|---|
| Target | Type | (SEQ ID NO) | (SEQ ID NO) |
| V. parahaemolyticus / | Primer | 58 | 58 |
| V. vulnificus | Primer | 21 | 20 |
| | Probe | 15 | 15 |
| | Probe | 31 | 31 |
| V. cholerae | Primer | 32 | 32 |
| | Primer | 3 | 3 |
| | Probe | 35 | 35 |

TABLE 13
Individual System Oligos

| | | PPR3 | PPR4 | PPR5 |
|---|---|---|---|---|
| | Oligo | (SEQ | (SEQ | (SEQ |
| Target | Type | ID NO) | ID NO) | ID NO) |
| V. parahaemolyticus | Primer | 53 | 53 | 53 |
| | Primer | 75 | 75 | 75 |
| | Probe | 33 | 33 | 33 |
| V. vulnificus | Primer | 42 | 55 | 43 |
| | Primer | 37 | 37 | 78 |
| | Probe | 9 | 9 | 31 |
| V. cholerae | Primer | 32 | 32 | 32 |
| | Primer | 3 | 3 | 3 |
| | Probe | 35 | 35 | 35 |

Results are shown in Table 14α-14c below.

TABLE 14a
V. vulnificus Amplification and Detection Results

| Conc. | | | | |
|---|---|---|---|---|
| (CFU/mL) | PPR | N | Ct Mean | Std Dev |
| 1.00E+02 | Dual Set | 0 | — | — |
| | Dual Set Mod RP | 3 | 38.82 | 0.41 |
| | Vvul Set 1 | 2 | 38.48 | 0.96 |
| | Vvul Set 2 | 3 | 36.65 | 1.70 |
| | Vvul Set 3 | 2 | 37.79 | 1.04 |
| 1.00E+03 | Dual Set | 0 | — | — |
| | Dual Set Mod RP | 3 | 34.39 | 0.09 |
| | Vvul Set 1 | 3 | 33.85 | 0.27 |
| | Vvul Set 2 | 3 | 34.07 | 0.39 |
| | Vvul Set 3 | 3 | 35.15 | 0.40 |
| 1.00E+04 | Dual Set | 2 | 43.66 | 1.12 |
| | Dual Set Mod RP | 3 | 31.78 | 0.18 |
| | Vvul Set 1 | 3 | 31.34 | 0.03 |
| | Vvul Set 2 | 3 | 31.39 | 0.24 |
| | Vvul Set 3 | 3 | 31.85 | 0.29 |

TABLE 14b
V. cholerae Amplification and Detection Results

| Conc. | | | | |
|---|---|---|---|---|
| (CFU/mL) | PPR | N | Ct Mean | Std Dev |
| 1.00E+02 | Dual Set | 3 | 36.81 | 1.26 |
| | Dual Set Mod RP | 3 | 37.25 | 1.68 |
| | Vvul Set 1 | 3 | 37.48 | 1.12 |
| | Vvul Set 2 | 3 | 36.43 | 0.96 |
| | Vvul Set 3 | 3 | 37.08 | 0.49 |
| 1.00E+03 | Dual Set | 3 | 33.70 | 0.14 |
| | Dual Set Mod RP | 3 | 33.65 | 0.32 |
| | Vvul Set 1 | 3 | 33.36 | 0.25 |
| | Vvul Set 2 | 3 | 32.94 | 0.74 |
| | Vvul Set 3 | 3 | 33.75 | 0.18 |
| 1.00E+04 | Dual Set | 3 | 30.13 | 0.08 |
| | Dual Set Mod RP | 3 | 30.07 | 0.23 |
| | Vvul Set 1 | 3 | 30.04 | 0.01 |
| | Vvul Set 2 | 3 | 29.92 | 0.19 |
| | Vvul Set 3 | 3 | 29.97 | 0.13 |

TABLE 14c
V. parahaemolyticus Amplification and Detection Results

| Conc. | | | | |
|---|---|---|---|---|
| (CFU/mL) | PPR | N | Ct Mean | Std Dev |
| 1.00E+02 | Dual Set | 3 | 37.93 | 0.26 |
| | Dual Set Mod RP | 3 | 39.53 | 0.90 |
| | Vvul Set 1 | 3 | 35.66 | 0.22 |
| | Vvul Set 2 | 3 | 36.15 | 0.16 |
| | Vvul Set 3 | 3 | 36.59 | 0.48 |
| 1.00E+03 | Dual Set | 3 | 34.47 | 0.25 |
| | Dual Set Mod RP | 3 | 35.79 | 0.55 |
| | Vvul Set 1 | 3 | 33.25 | 0.15 |
| | Vvul Set 2 | 3 | 32.78 | 0.37 |
| | Vvul Set 3 | 3 | 33.17 | 0.72 |
| 1.00E+04 | Dual Set | 3 | 31.43 | 0.26 |
| | Dual Set Mod RP | 3 | 30.67 | 0.10 |
| | Vvul Set 1 | 3 | 29.65 | 0.40 |
| | Vvul Set 2 | 3 | 29.10 | 0.08 |
| | Vvul Set 3 | 3 | 29.56 | 0.43 |

All Vibrio vulnificus systems amplified V. vulnificus in this test set (except the original Set 1 mismatch PPR1). The modification to the V. parahaemolyticus dual reverse primer rescued the performance of the V. vulnificus detection in the dual system (PPR2 vs PPR1). The dual system amplifies well for both V. parahaemolyticus and V. vulnificus, but slightly earlier Cts at 1e3 and 1e2 CFU/mL are seen with the individual systems for both V. parahaemolyticus and V. vulnificus (PPR 2 vs PPR3,4,5). The V. cholerae system performs well with both the dual system and individual systems for V. parahaemolyticus and V. vulnificus.

### Example 7: Y. enterocolitica, Vibrio Sun., STEC O157. And P. shigelloides Limit of Detection in Multiplex System

A small-scale limit of detection (LoD) study was conducted to determine appropriate concentrations for probit analysis. Panels built in raw stool were tested with using a multiplex oligo set at three replicates each with 3e2, 1e3, 3e1, and 1e1 CFU/mL concentrations for all targets: Y. enterocolitica. V. cholerae, V. parahaemolyticus, V. vulnificus, P. shigelloides, and STEC O157. The primers and probes used in the experiment are shown in Table 15.

TABLE 15

| | Oligo | SEQ ID | |
|---|---|---|---|
| Target | Type | NO | Modifications |
| V. parahaemolyticus | Primer | 53 | |
| | Primer | 75 | |
| | Probe | 33 | CalOrange560/BHQ1 |
| | | | Reverse polarity C |
| V. vulnificus | Primer | 55 | |
| | Primer | 37 | |
| | Probe | 9 | CalOrange560/BHQ1 |
| | | | Reverse polarity C |
| V. cholerae | Primer | 32 | |
| | Primer | 3 | |
| | Probe | 35 | CalOrange560/BHQ1 |
| | | | Reverse polarity C |
| STEC O157 | Primer | 71 | |
| | Primer | 44 | 5 mC at residues 2, 15, 16, and 24 |
| | | | pdU at residue 20 |
| | Probe | 74 | 5 mC at residues 3, 6, 13, 14, 19, and 22 |
| | | | pdU at residues 4, 9, 17, and 20 |
| | | | CalRed610/BHQ2 |
| | Probe | 7 | 5 mC at residues 2, 5, 6, and 20 |
| | | | pdU at residues 4, 8, 10, 13, 15, and 24 |
| | | | CalRed610/BHQ2 |
| Y. enterocolitica | Primer | 6 | |
| | Primer | 24 | |
| | Probe | 63 | FAM/BHQ1 |
| | | | Reverse polarity C |
| P. shigelloides | Primer | 17 | |
| | Primer | 1 | |
| | Probe | 61 | Quasar670/BHQ2 |
| | Probe | 72 | Quasar670/BHQ2 |
| Internal Control (IC) | Primer | 23 | |
| | Primer | 38 | |
| | Probe | 36 | Quasar705/BHQ2 |

Results are shown in Table 16 below.

TABLE 16

| | Target | | | | |
|---|---|---|---|---|---|
| | Conc | Target Channel | | Quasar 705 (IC) | |
| Target | (CFU/mL) | Avg Ct | Avg RFU | Avg Ct | Avg RFU |
| Y. enterocolitica | 1E1 | 37.55 | 4126 | 30.23 | 6979 |
| | 3E1 | 36.39 | 9501 | 30.16 | 7072 |
| | 1E2 | 34.63 | 17902 | 29.92 | 7850 |
| | 3E2 | 33.76 | 21151 | 30.37 | 6217 |
| V. cholerae | 1E1 | — | 7 | 30.11 | 6858 |
| | 3E1 | — | 88 | 30.29 | 5611 |
| | 1E2 | 43.44 | 445 | 29.71 | 8097 |
| | 3E2 | 38.57 | 932 | 29.90 | 8374 |
| V. | 1E1 | — | 160 | 29.93 | 8397 |
| parahaemolyticus | 3E1 | — | 232 | 29.87 | 7537 |
| | 1E2 | 38.99 | 784 | 30.18 | 7146 |
| | 3E2 | 38.33 | 919 | 30.13 | 5951 |
| V. vulnificus | 1E1 | — | 59 | 30.00 | 7361 |
| | 3E1 | — | 67 | 29.93 | 8307 |
| | 1E2 | 41.47 | 393 | 30.05 | 7504 |
| | 3E2 | 38.28 | 1048 | 30.07 | 7450 |
| STEC O157 | 1E1 | 42.06 | 301 | 30.00 | 7834 |
| | 3E1 | 41.86 | 458 | 30.28 | 6534 |
| | 1E2 | 37.93 | 1485 | 30.18 | 6786 |
| | 3E2 | 35.51 | 1737 | 30.27 | 5641 |
| P. shigelloides | 1E1 | 39.95 | 508 | 29.88 | 8169 |
| | 3E1 | 39.57 | 524 | 29.54 | 7363 |
| | 1E2 | 39.19 | 780 | 30.24 | 6420 |
| | 3E2 | 36.55 | 1860 | 30.20 | 7700 |
| Negative Cntrl | — | | | 30.09 | 6472 |

Final LoD determinations are shown in Table 17 below. More replicates will be needed to calculate confidence intervals.

TABLE 17
Target Limit of Detection

| Target | Raw Stool LoD in CFU/mL | McFadden R2 |
|---|---|---|
| Vibrio parahaemolyticus | 75 | 0.714 |
| Vibrio vulnificus | 146 | 0.594 |
| Vibrio cholerae | 43 | 0.840 |
| Yersinia enterocolitica | 12 | 0.840 |
| Plesiomonas shigelloides | 12 | 0.840 |
| STEC O157 | 75 | 0.714 |

### Example 8: Amplification and Detection of Y. enterocolitica and P. shigelloides Targets in Multiplex Systems

Primer and probe sets for Yersinia enterocolitica (ail gene) and Plesiomonas shigelloides (hugA gene) (two sets of oligos each) were tested in multiplex using lysate in STM panels (1E6 and 1E4 CFU/mL). The primers and probes used in the experiment are shown in Table 18.

TABLE 18

| | Oligo | SEQ ID | |
|---|---|---|---|
| Oligo Set | Type | NO | Modifications |
| Y. enterocolitica | Primer | 10 | |
| Set 1 | Primer | 4 | |
| | Probe | 50 | FAM/BHQ1 |
| | | | Reverse polarity C |
| Y. enterocolitica | Primer | 6 | |
| Set 2 | Primer | 24 | |
| | Probe | 63 | FAM/BHQ1 |
| | | | Reverse polarity C |
| P. shigelloides | Primer | 17 | |
| Set 1 | Primer | 1 | |
| | Probe | 72 | Quasar670/BHQ2 |
| P. shigelloides | Primer | 79 | |
| Set 2 | Primer | 68 | |
| | Probe | 8 | Quasar670/BHQ2 |

Four different multiplex systems were tested:
A) Y. enterocolitica Set 1+P. shigelloides Set 1
B) Y. enterocolitica Set 1+P. shigelloides Set 2
C) Y. enterocolitica Set 2+P. shigelloides Set 1
D) Y. enterocolitica Set 2+P. shigelloides Set 2.

Each multiplex system included an internal control (IC) primer and probe set (primers of SEQ ID NOs:23 and 38 and probe of SEQ ID NO:36). The IC probe was labeled with Quasar 705 and BHQ2.

Results are shown in Table 19 below.

TABLE 19

| | Average of Ct | | | | | | | |
|---|---|---|---|---|---|---|---|---|
| | FAM | | | | Quasar 670 | | | |
| Sample ID | Setup 1 | Setup 2 | Setup 3 | Setup 4 | Setup 1 | Setup 2 | Setup 3 | Setup 4 |
| Pshig_1E4_CFU | NA | NA | NA | NA | 30.33 | 28.87 | 30.64 | 28.84 |
| Pshig_1E6_CFU | NA | NA | NA | NA | 23.66 | 21.99 | 23.64 | 22.19 |
| Yent_1E4_CFU | 28.04 | 28.13 | 27.72 | 27.42 | NA | NA | NA | NA |
| Yent_1E6_CFU | 21.47 | 21.85 | 21.08 | 20.90 | NA | NA | NA | NA |
| | Average of RFU | | | | | | | |
| | FAM | | | | Quasar 670 | | | |
| Sample ID | Setup 1 | Setup 2 | Setup 3 | Setup 4 | Setup 1 | Setup 2 | Setup 3 | Setup 4 |
| Pshig_1E4_CFU | NA | NA | NA | NA | 6297 | 10605 | 5479 | 11744 |
| Pshig_1E6_CFU | NA | NA | NA | NA | 6857 | 21589 | 6645 | 22396 |
| Yent_1E4_CFU | 37134 | 32363 | 41196 | 48117 | NA | NA | NA | NA |
| Yent_1E6_CFU | 39316 | 35180 | 50184 | 47401 | NA | NA | NA | NA |

All oligo sets worked well in multiplex of the two targets. Multiplex system C looked the best for both at the tested concentrations and will be tested in the full multiplex.

### Example 9: Plesiomonas shigelloides Probe Comparison

Due to the low level of fluorescence in the Quasar 670 channel for Plesiomonas (using a probe of SEQ ID NO:70 with primers of SEQ ID NO:17 and SEQ ID NO:1), a second probe (SEQ ID NO:61) was designed to boost its detection level. Each of the probes (labeled with Quasar 670 and BHQ2) was tested separately and combined together with the SEQ ID NO:17/SEQ ID NO: 1 primers to compare their performance. Target was tested as lysate panels in STM at 1e6, 1e4, 1e3, 1e2, and 1e1 CFU/mL at three replicates each. Each oligo set included an internal control (IC) primer and probe set (primers of SEQ ID NOs:23 and 38 and probe of SEQ ID NO:36 labeled with Quasar 705 and BHQ2).

Results are shown in Tables 20 and 21 below.

TABLE 20
Mean RFU for P. shigelloides Probe Comparison

| | Probe Condition | | |
|---|---|---|---|
| Target Conc | SEQ ID NO: 70 | SEQ ID NO: 70 / | SEQ ID NO: 61 |
| (CFU/mL) | only | SEQ ID NO: 61 | only |
| 1E1 | 1496 | 2389 | 605 |
| 1E2 | 2672 | 3219 | 1462 |
| 1E3 | 2958 | 4827 | 2260 |
| 1E4 | 3416 | 5530 | 2481 |
| 1E6 | 3402 | 4772 | 2557 |

TABLE 21
Mean Ct for P. shigelloides Probe Comparison

| | Probe Condition | | |
|---|---|---|---|
| Target Conc | SEQ ID NO: 70 | SEQ ID NO: 70 / | SEQ ID NO: 61 |
| (CFU/mL) | only | SEQ ID NO: 61 | only |
| 1E1 | 41.3 | 40.38 | 43.15 |
| 1E2 | 38.36 | 39.04 | 40.62 |
| 1E3 | 35.45 | 35.02 | 36.7 |
| 1E4 | 32.17 | 31.21 | 33.43 |
| 1E6 | 25.46 | 25.09 | 27.01 |

Each probe demonstrated similar level of detection separately (3000-3500 RFU). Both in combination demonstrated a higher RFU value of5500.

### Example 10: Primer and Probe Titration

The objective of this study was to lower the concentration of primers and probes from the nominal concentrations of30.7 μM for primers and 0.5 M for probes. Three different conditions were tested against the nominal condition for comparison. Lysate panels were tested at 1E2 and 1E3 CFU/mL.

The primers and probes used in the experiment are shown in Table 22.

TABLE 22

| | Oligo | SEQ ID | |
|---|---|---|---|
| Target | Type | NO | Modifications |
| V. parahaemolyticus | Primer | 53 | |
| | Primer | 75 | |
| | Probe | 33 | CalOrange560/BHQ1 |
| | | | Reverse polarity C |
| V. vulnificus | Primer | 55 | |
| | Primer | 37 | |
| | Probe | 9 | CalOrange560/BHQ1 |
| | | | Reverse polarity C |
| V. cholerae | Primer | 32 | |
| | Primer | 3 | |
| | Probe | 35 | CalOrange560/BHQ1 |
| | | | Reverse polarity C |
| STEC O157 | Primer | 71 | |
| | Primer | 44 | 5 mC at residues 2, 15, 16, and 24 |
| | | | pdU at residue 20 |
| | Probe | 74 | 5 mC at residues 3, 6, 13, 14, 19, and 22 |
| | | | pdU at residues 4, 9, 17, and 20 |
| | | | CalRed610/BHQ2 |
| Y. enterocolitica | Primer | 6 | |
| | Primer | 24 | |
| | Probe | 63 | FAM/BHQ1 |
| | | | Reverse polarity C |
| P. shigelloides | Primer | 17 | |
| | Primer | 1 | |
| | Probe | 61 | Quasar670/BHQ2 |
| Internal Control (IC) | Primer | 23 | |
| | Primer | 38 | |
| | Probe | 36 | Quasar705/BHQ2 |

Concentrations tested are shown in Table 23 below.

TABLE 23

| | Primer conc | Probe conc |
|---|---|---|
| Condition | (μM) | (μM) |
| A | 0.7 | 0.5 |
| B | 0.7 | 0.375 |
| C | 0.7 | 0.25 |
| D | 0.5 | 0.375 |

Results. Lowering the concentration of probe to 0.25 or 0.375 M increased the level of detection for some channels and lowered the background noise. Ct values were similar across all conditions tested. Oligo concentrations can be lowered to 0.5/0.25 without changing the performance of the system in comparison to the nominal condition.

### Example 11: Oligonucleotide Modifications

Some targets demonstrated a fanning effect in their linearity amplification curves. To improve the shape of the curves, a modification to the oligos was done by deleting/adding bases for better specificity. Testing was done with plasmid panels then checked with lysate to test performance. The oligonucleotide modifications are shown in Table 24.

TABLE 24

| | | Original | Modified | |
|---|---|---|---|---|
| | Oligo | Sequence | Sequence | |
| Target | Type | (SEQ ID NO) | (SEQ ID NO) | Change |
| V. cholerae | Primer | 32 | 85 | Removed 1st and 2nd 5′ C |
| V. cholerae | Primer | 3 | 105 | Removed 3′ T |
| V. parahaemolyticus | Primer | 53 | 52 | Removed 3′ T |
| STEC O157 | Primer | 71 | 70 | Removed 3′ AAA |
| STEC O157 | Probe | 74 | 73 | Removed 3′ AAT |
| V. vulnificus | Primer | 37 | 47 | Removed 5′ C, added 3′ AG |
| P. shigelloides | Primer | 1 | 106 | Removed 5′ A and 3′ CC |

The primers and probes used in the experiment are shown in Table 25.

TABLE 25

| | Oligo | SEQ ID NO | SEQ ID NO | |
|---|---|---|---|---|
| Target | Type | (Old Set) | (New Set) | Modifications |
| V. | Primer | 53 | 52 | |
| parahaemolyticus | Primer | 75 | 75 | |
| | Probe | 33 | 33 | CalOrange560/BHQ1 |
| | | | | Reverse polarity C |
| V. vulnificus | Primer | 55 | 55 | |
| | Primer | 37 | 47 | |
| | Probe | 9 | 9 | CalOrange560/BHQ1 |
| | | | | Reverse polarity C |
| V. cholerae | Primer | 32 | 85 | |
| | Primer | 3 | 105 | |
| | Probe | 35 | 35 | CalOrange560/BHQ1 |
| | | | | Reverse polarity C |
| STEC O157 | Primer | 71 | 70 | |
| | Primer | 44 | 44 | 5 mC at residues 2, 15, 16, and |
| | | | | 24 |
| | | | | pdU at residue 20 |
| | Probe | 74 | 73 | 5 mC at residues 3, 6, 13, 14, 19, |
| | | | | and 22 |
| | | | | pdU at residues 4, 9, 17, and 20 |
| | | | | (SEQ ID NO: 74) or at residues |
| | | | | 2, 4, 7, 8, 9, 10, 17, 18, and 20 |
| | | | | (SEQ ID NO: 73) |
| | | | | CalRed610/BHQ2 |
| Y. enterocolitica | Primer | 6 | 6 | |
| | Primer | 24 | 24 | |
| | Probe | 63 | 63 | FAM/BHQ1 |
| | | | | Reverse polarity C |
| P. shigelloides | Primer | 17 | 17 | |
| | Primer | 1 | 106 | 5 mC at residues 6 and 21 (SEQ |
| | | | | ID NO: 106) |
| | Probe | 61 | 61 | Quasar670/BHQ2 |
| Internal Control | Primer | 23 | 23 | |
| (IC) | Primer | 38 | 38 | |
| | Probe | 36 | 36 | Quasar705/BHQ2 |

Results are shown in Table 26 below.

TABLE 26

| Sample ID | Avg Ct | Avg RFU |
|---|---|---|
| O157_1.7E3_cp/mL | 31.74 | 24201 |
| O157_2.5E3_cp/mL | 31.29 | 27173 |
| O157_3.3E1_cp/mL | NA | NA |
| O157_3.3E2_cp/mL | NA | NA |
| O157_3.3E3_cp/mL | 30.85 | 28054 |
| O157_3.3E4_cp/mL | 27.33 | 33213 |
| Pshg_1.7E3_cp/mL | 33.40 | 10311 |
| Pshg_2.5E3_cp/mL | 32.95 | 10724 |
| Pshg_3.3E1_cp/mL | NA | NA |
| Pshg_3.3E2_cp/mL | 35.81 | 7649 |
| Pshg_3.3E3_cp/mL | 32.54 | 11127 |
| Pshg_3.3E4_cp/mL | 29.50 | 13151 |
| Pshg_8.3E2_cp/mL | 34.54 | 9388 |
| Vchl_1.7E3_cp/mL | 33.98 | 3445 |
| Vchl_2.5E3_cp/mL | 33.32 | 3883 |
| Vchl_3.3E1_cp/mL | NA | NA |
| Vchl_3.3E2_cp/mL | 37.04 | 1880 |
| Vchl_3.3E3_cp/mL | 33.01 | 2947 |
| Vchl_3.3E4_cp/mL | 29.33 | 7313 |
| Vpar_1.7E3_cp/mL | 34.34 | 3085 |
| Vpar_2.5E3_cp/mL | 33.14 | 5636 |
| Vpar_3.3E1_cp/mL | 39.38 | 1114 |
| Vpar_3.3E2_cp/mL | 35.94 | 3040 |
| Vpar_3.3E3_cp/mL | 32.86 | 5204 |
| Vpar_3.3E4_cp/mL | 29.55 | 7233 |
| Vpar_8.3E2_cp/mL | 34.72 | 4734 |
| Vvul_1.7E3_cp/mL | 34.28 | 4128 |
| Vvul_2.5E3_cp/mL | 33.37 | 4321 |
| Vvul_3.3E1_cp/mL | 40.55 | 1511 |
| Vvul_3.3E2_cp/mL | 38.74 | 1827 |
| Vvul_3.3E3_cp/mL | 33.04 | 4567 |
| Vvul_3.3E4_cp/mL | 29.77 | 5654 |
| Vvul_8.3E2_cp/mL | 35.37 | 2974 |
| Yent_3.3E1_cp/mL | NA | NA |
| Yent_3.3E2_cp/mL | 35.56 | 21540 |
| Yent_3.3E3_cp/mL | 32.15 | 35296 |
| Yent_3.3E4_cp/mL | 28.52 | 39978 |
| Yent_3.3E5_cp/mL | 25.48 | 39541 |

Modifying some of the oligos improved the amplification curve shape and overall performance of each target.

### Example 12: Y. enterocolitica, Vibrio Spp., STEC O157, and P. shigelloides Inclusivity in Multiplex System

Primer and probe sets for detection of Yersinia enterocolitica, Vibrio spp., STEC O157, and Plesiomonas shigelloides were tested in multiplex against different strains for each bacterial target. All targets were tested as lysate panels for each inclusivity strain at 3× and 10×LoD at three replicates each (see LoD Summary in Table 27). The primers and probes used in the experiment are shown in Table 28.

TABLE 27
LoD Summary

| | Concentration | | |
|---|---|---|---|
| Species | (CFU/mL) | 3x LoD | 10x LoD |
| Y. enterocolitica | 30 | 90 | 300 |
| V. parahaemolyticus | 70 | 210 | 700 |
| V. cholerae | 30 | 90 | 300 |
| V. vulnificus | 300 | 900 | 3000 |
| STEC O157 | 300 | 900 | 3000 |
| P. shigelloides | 70 | 210 | 700 |

TABLE 28

| | Oligo | SEQ ID | |
|---|---|---|---|
| Target | Type | NO | Modifications |
| V. parahaemolyticus | Primer | 51 | |
| | Primer | 76 | pdU at residues 2 and 10 |
| | Probe | 33 | CalOrange560/BHQ1 |
| | | | Reverse polarity C |
| V. vulnificus | Primer | 66 | pdU at residues 6 and 10 |
| | Primer | 62 | 5 mC at residues 3, 6, 9, and 16 |
| | | | pdU at residues 1, 8, 12, 15, 17, and 18 |
| | Probe | 9 | CalOrange560/BHQ1 |
| | | | Reverse polarity C |
| V. cholerae | Primer | 56 | |
| | Primer | 11 | 5 mC at residues 8, 10, and 17 |
| | | | pdU at residues 3, 4, 5, 7, 11, 14, 16, 19, and 20 |
| | Probe | 12 | 5 mC at residue 20 |
| | | | pdU at residues 7, 13, 15, 16 and 18 |
| | | | CalOrange560/BHQ1 |
| | | | Reverse polarity C |
| STEC O157 | Primer | 70 | 5 mC at residues 3, 6, 8, 12, and 15 |
| | Primer | 16 | 5 mC at residue 16 |
| | | | pdU at residues 4, 6, 7, 9, 11, 13, 15, 19, 20, 21, |
| | | | and 23 |
| | Probe | 73 | 5 mC at residues 3, 6, 13, 14, 19, and 22 |
| | | | pdU at residues 2, 4, 7, 8, 9, 10, 17, 18, and 20 |
| | | | CalRed610/BHQ2 |
| Y. enterocolitica | Primer | 65 | |
| | Primer | 54 | |
| | Probe | 18 | 5 mC at residues 5, 10, 18, and 24 |
| | | | pdU at residues 8 and 16 |
| | | | FAM/BHQ1 |
| | | | Reverse polarity C |
| P. shigelloides | Primer | 2 | |
| | Primer | 49 | |
| | Probe | 48 | 5 mC at residues 3, 7, 8, 12, and 21 |
| | | | pdU at residues 5, 6, 9, 14, and 18 |
| | | | Quasar670/BHQ2 |
| Internal Control (IC) | Primer | 69 | |
| | Primer | 39 | |
| | Probe | 14 | Quasar705/BHQ2 |

Results are shown in Table 29 below.

TABLE 29

| | | | | Target Channel | | | Quasar 705 (IC) | | |
|---|---|---|---|---|---|---|---|---|---|
| | | | Conc | | Avg | Avg | | Avg | Avg |
| Target | GP# | ATCC# | (CFU/mL) | Reactivity | Ct | RFU | Reactivity | Ct | RFU |
| Negative | N/A | N/A | 0 | | | | 3/3 | 25.77 | 27510 |
| Y. ent | GP2387 | BEI NR-207 | 90 | 2/3 | 38.71 | 5774 | 3/3 | 24.91 | 39487 |
| | | | 150 | 3/3 | 37.08 | 7471 | 3/3 | 25.86 | 38781 |
| | GP2388 | BEI NR-212 | 90 | 3/3 | 35.66 | 11614 | 3/3 | 24.99 | 39075 |
| | GP2398 | ATCC | 90 | 3/3 | 37.04 | 8019 | 3/3 | 25.01 | 38831 |
| | | 23715 | | | | | | | |
| | GP2399 | ATCC | 90 | 3/3 | 34.76 | 23369 | 3/3 | 25.73 | 27596 |
| | | 49397 | | | | | | | |
| | GP2430 | CCUG 4588 | 90 | 3/3 | 37.01 | 8063 | 3/3 | 25.42 | 38361 |
| | GP2431 | CCUG 8050 | 90 | 3/3 | 35.58 | 12197 | 3/3 | 24.82 | 38057 |
| | GP2432 | CCUG 8232 | 90 | 3/3 | 35.25 | 14425 | 3/3 | 24.93 | 38658 |
| | GP2433 | CCUG 8234 | 90 | 3/3 | 36.37 | 10359 | 3/3 | 24.98 | 38167 |
| | GP2436 | ATCC | 90 | 3/3 | 34.99 | 23477 | 3/3 | 25.75 | 27854 |
| | | 55075 | | | | | | | |
| | GP2441 | NCTC | 90 | 3/3 | 35.93 | 18940 | 3/3 | 25.74 | 26337 |
| | | 10463 | | | | | | | |
| | GP2498 | ATCC27729 | 90 | 3/3 | 36.71 | 10522 | 3/3 | 24.99 | 39439 |
| EO157 | GP1578 | 700377 | 900 | 3/3 | 30.84 | 56806 | 3/3 | 25.70 | 29448 |
| | GP1585 | 700927 | 900 | 3/3 | 31.29 | 55561 | 3/3 | 25.57 | 29963 |
| | GP1587 | 43894 | 900 | 3/3 | 30.49 | 59269 | 3/3 | 25.36 | 30752 |
| | GP1589 | 700378 | 900 | 3/3 | 31.04 | 56776 | 3/3 | 25.55 | 29533 |
| | GP2290 | 700375 | 900 | 3/3 | 30.18 | 63015 | 3/3 | 25.55 | 29718 |
| | GP2344 | ATCC | 900 | 3/3 | 32.13 | 51654 | 3/3 | 25.64 | 29522 |
| | | 43890/ CDC | | | | | | | |
| | | C984 | | | | | | | |
| | GP2345 | ATCC | 900 | 3/3 | 31.50 | 56886 | 3/3 | 25.51 | 29729 |
| | | 43895/ CDC | | | | | | | |
| | | EDL 933 | | | | | | | |
| P. shig | GP2395 | ATCC | 210 | 3/3 | 33.06 | 25327 | 3/3 | 25.06 | 40588 |
| | | 14030 | | | | | | | |
| | GP2396 | ATCC | 210 | 3/3 | 35.12 | 27596 | 3/3 | 25.97 | 25744 |
| | | 51572 | | | | | | | |
| | GP2397 | ATCC | 210 | 3/3 | 35.84 | 29661 | 3/3 | 25.80 | 29150 |
| | | 51903 | | | | | | | |
| | GP2426 | CCUG 7041 | 210 | 3/3 | 36.21 | 26995 | 3/3 | 25.72 | 28650 |
| | | A | | | | | | | |
| | GP2427 | CCUG 9221 | 210 | 3/3 | 35.28 | 31894 | 3/3 | 25.84 | 28544 |
| | GP2428 | CCUG | 210 | 3/3 | 34.56 | 24639 | 3/3 | 24.89 | 41312 |
| | | 14309 | | | | | | | |
| | GP2429 | CCUG | 210 | 3/3 | 34.92 | 25013 | 3/3 | 24.88 | 42247 |
| | | 14597 | | | | | | | |
| V. chol | GP2391 | BEI NR-147 | 90 | 3/3 | 33.11 | 41030 | 3/3 | 25.51 | 29418 |
| | GP2392 | BEI NR-148 | 90 | 3/3 | 32.89 | 43103 | 3/3 | 25.68 | 29451 |
| | GP2393 | BEI NR-149 | 90 | 3/3 | 31.46 | 51448 | 3/3 | 25.63 | 28801 |
| | GP2394 | BEI NR-152 | 90 | 3/3 | 32.64 | 44869 | 3/3 | 25.58 | 29673 |
| | GP2410 | ATCC | 90 | 3/3 | 33.14 | 41729 | 3/3 | 25.53 | 29230 |
| | | 14033 | | | | | | | |
| | GP2411 | ATCC 9458 | 90 | 3/3 | 32.17 | 46365 | 3/3 | 25.62 | 29190 |
| | GP2412 | ATCC 9459 | 90 | 3/3 | 32.55 | 43582 | 3/3 | 25.44 | 30099 |
| | GP2511 | CCUG2573 | 90 | 3/3 | 33.13 | 45533 | 3/3 | 25.55 | 30146 |
| | GP2512 | CCUG2569 | 90 | 3/3 | 34.12 | 35009 | 3/3 | 25.50 | 30166 |
| | GP2513 | CCUG4070 | 90 | 3/3 | 32.89 | 37828 | 3/3 | 25.71 | 29759 |
| | GP2514 | CCUG21589 | 90 | 3/3 | 32.25 | 50917 | 3/3 | 25.68 | 30297 |
| | GP2515 | CCUG56875 | 90 | 3/3 | 32.31 | 46678 | 3/3 | 25.50 | 31428 |
| | GP2516 | CCUG53725 | 90 | 3/3 | 33.02 | 42854 | 3/3 | 25.60 | 30067 |
| | GP2517 | CCUG14542 | 90 | 3/3 | 32.65 | 49674 | 3/3 | 25.44 | 30232 |
| V. para | GP2389 | BEI NR- | 210 | 3/3 | 30.02 | 71736 | 3/3 | 25.68 | 28846 |
| | | 21990 | | | | | | | |
| | GP2390 | BEI NR- | 210 | 3/3 | 30.10 | 79782 | 3/3 | 25.65 | 28843 |
| | | 21992 | | | | | | | |
| | GP2400 | ATCC | 210 | 3/3 | 30.11 | 81697 | 3/3 | 25.58 | 28857 |
| | | BAA-242 | | | | | | | |
| | GP2401 | ATCC | 210 | 3/3 | 28.42 | 91057 | 3/3 | 25.54 | 28981 |
| | | 27969 | | | | | | | |
| | GP2402 | ATCC | 210 | 3/3 | 31.52 | 57106 | 3/3 | 25.57 | 29832 |
| | | 33845 | | | | | | | |
| | GP2403 | ATCC | 210 | 3/3 | 30.90 | 69557 | 3/3 | 25.48 | 29875 |
| | | BAA-241 | | | | | | | |
| | GP2404 | ATCC | 210 | 3/3 | 30.94 | 54048 | 3/3 | 25.43 | 30395 |
| | | 49529 | | | | | | | |
| | GP2405 | ATCC | 210 | 3/3 | 31.12 | 64872 | 3/3 | 25.56 | 29882 |
| | | 43996 | | | | | | | |
| | GP2406 | ATCC | 210 | 3/3 | 31.41 | 47882 | 3/3 | 25.61 | 29667 |
| | | 33846 | | | | | | | |
| | GP2437 | ATCC | 210 | 3/3 | 32.22 | 36660 | 3/3 | 25.60 | 30244 |
| | | 33847 | | | | | | | |
| | GP2439 | CCUG | 210 | 3/3 | 31.93 | 52452 | 3/3 | 25.53 | 29108 |
| | | 67711 | | | | | | | |
| | GP2440 | CCUG | 210 | 3/3 | 30.46 | 73999 | 3/3 | 25.31 | 30904 |
| | | 34902 | | | | | | | |
| V. vul | GP2407 | ATCC | 900 | 3/3 | 31.28 | 40413 | 3/3 | 25.67 | 28457 |
| | | 33817 | | | | | | | |
| | GP2408 | ATCC | 900 | 3/3 | 31.62 | 31545 | 3/3 | 25.74 | 28266 |
| | | BAA-86 | | | | | | | |
| | GP2434 | CCUG | 900 | 3/3 | 31.71 | 35010 | 3/3 | 25.60 | 28865 |
| | | 38297 | | | | | | | |
| | GP2435 | CCUG | 900 | 3/3 | 32.78 | 16516 | 3/3 | 25.57 | 28848 |
| | | 47321 | | | | | | | |
| | GP2438 | ATCC | 900 | 3/3 | 32.09 | 35929 | 3/3 | 25.56 | 29742 |
| | | 29306 | | | | | | | |

All inclusivity strains yielded 100% positivity at their respective 3×LoD with the exception of Yersinia GP2387, which passed at 5×LoD.

### Example 13: Evaluation of Yersinia enterocolitica InvA Gene for Amplification and Detection

The tests listed in Table 30 were performed to evaluate oligos targeting the InvA gene for Yersinia enterocolitica detection.

TABLE 30
Experiments Conducted

| Experiment | Purpose | Primers/Probes |
|---|---|---|
| A | Screening | Monoplex |
| B | Inclusivity | Monoplex |
| C | All Targets* | Multiplex |
| D | LoD | Multiplex |

*Y. enterocolitica, V. parahaemolyticus, V. vulnificus, V. cholerae, STEC O157, and P. shigelloides

Target was tested as lysate panels in STM. For Experiment A, target was tested at 70, 30, and 10 CFU/mL at three replicates each. For Experiment B, 100×LoD panels (11 strains as shown in Table 31) built in a prior experiment, along with NY Bio 199 and 204 clinical samples, were used at one replicate each. For Experiment C, targets were tested at 1E3 CFU/mL at three replicates each. For Experiment D, targets were tested at the concentrations shown in Table 32 at two replicates each.

TABLE 31
Yersinia enterocolitica Strains for Experiment B

| ID | Item | Strain/variant | Vendor ID | Vendor |
|---|---|---|---|---|
| GP2387 | Yersinia enterocolitica, | CDC 497-70 | NR-207 | BEI Resources |
| | O: 8 | | | |
| GP2388 | Yersinia enterocolitica, | NCTC 11175; Serovar 3 | NR-212 | BEI Resources |
| | O: 3 | | | |
| GP2398 | Yersinia enterocolitica, | Biotype 1; Serotype 8 | ATCC 23715 | America Type Culture |
| | O: 8 | | | Collection |
| GP2399 | Yersinia enterocolitica, | 1375 | ATCC 49397 | America Type Culture |
| | O: 8 | | | Collection |
| GP2441 | Yersinia enterocolitica, | P 77; serovar 5 | NCTC 10463 | UK Health Security |
| | O: 5, 27 | | | Agency |
| GP2430 | Yersinia enterocolitica, | Biogroup 2; Serovar 9 | CCUG 4588 | Culture Collection |
| | O: 9 | | | University of Gothenburg |
| GP2431 | Yersinia enterocolitica | | CCUG 8050 | Culture Collection |
| | | | | University of Gothenburg |
| GP2432 | Yersinia enterocolitica, | Biotype 5; Serotype | CCUG 8232 | Culture Collection |
| | O: 1, 2, 3 \| O: 2, 3 \| | O: 1, 2, 3 \| O: 2, 3 \| | | University of Gothenburg |
| | O: 3/XI | O: 3/XI | | |
| GP2433 | Yersinia enterocolitica | | CCUG 8234 | Culture Collection |
| | | | | University of Gothenburg |
| GP2436 | Yersinia enterocolitica, | Biogroup 2; Serotype 9 | ATCC 55075 | America Type Culture |
| | O: 9 | | | Collection |
| GP2498 | Yersinia | Biotype 1; Serotype 8 | ATCC 27729 | America Type Culture |
| | enterocolytica, O: 8 | | | Collection |

TABLE 32
Target Concentrations for Experiment D

| Target species | Concentrations (CFU/mL) |
|---|---|
| Y. enterocolitica | 300, 100, 70, 30, 10, 3 |
| V. parahaemolyticus | 210, 70 |
| V. vulnificus | 900, 300 |
| V. cholerae | 90, 30 |
| STEC O157 | 1000, 90, 30 |
| P. shigelloides | 210, 70 |

The Yersinia primers and probes used in the experiment are shown in Table 33.

TABLE 33
Y. enterocolitica Primer and Probe Sets Targeting InvA

| Oligo | Oligo | SEQ | |
|---|---|---|---|
| Set | Type | ID NO | Modifications |
| 1 | Primer | 26 | |
| | Primer | 46 | 5 mC at residues 13 and 18 |
| | Probe | 40 | 5 mC at residues 7, 8, 9, 10, and 18 |
| | | | FAM/BHQ1 |
| | | | Reverse polarity C |
| 2 | Primer | 13 | |
| | Primer | 22 | Inosine at residue 6 |
| | Probe | 29 | 5 mC at residues 2, 3, 6, 8, and 20 |
| | | | pdU at residues 5, 17, and 23 |
| | | | FAM/BHQ1 |
| | | | Reverse polarity C |
| 3 | Primer | 77 | |
| | Primer | 41 | 5 mC at residues 5, 6, 11, and 22 |
| | | | pdU at residues 7, 12, 15, and 19 |
| | Probe | 34 | 5 mC at residues 7, 9, 20, and 24 |
| | | | Inosine at residue 23 |
| | | | FAM/BHQ1 |
| | | | Reverse polarity C |
| 4 | Primer | 28 | 5 mC at residues 2, 4, 6, 7, and 12 |
| | Primer | 30 | |
| | Probe | 27 | 5 mC at residues 2, 5, and 22 |
| | | | pdU at residues 10, 15, 17, 19, and 23 |
| | | | FAM/BHQ1 |
| | | | Reverse polarity C |
| 5 | Primer | 60 | |
| | Primer | 45 | 5 mC at residues 8 and 20 |
| | Probe | 25 | 5 mC at residues 9 and 13 |
| | | | pdU at residues 10, 15, 16, and 26 |
| | | | FAM/BHQ1 |
| | | | Reverse polarity C |
| Nominal | Primer | 65 | |
| (“Nom”) | Primer | 54 | |
| | Probe | 18 | 5 mC at residues 5, 10, 18, and 24 |
| | | | pdU at residues 8 and 16 |
| | | | FAM/BHQ1 |
| | | | Reverse polarity C |

Vibrio, STEC O157, and P. shigelloides primers and probes used in the experiment are shown in Table 34.

TABLE 34
Vibrio, STEC 0157, and P. shigelloides Primer and Probe Sets

| | Oligo | | |
|---|---|---|---|
| Target | Type | SEQ ID NO | Modifications |
| V. | Primer | 51 | |
| parahaemolyticus | Primer | 76 | pdU at residues 2 and 10 |
| | Probe | 33 | HEX/BHQ1 |
| | | | Reverse polarity C |
| V. vulnificus | Primer | 66 | pdU at residues 6 and 10 |
| | Primer | 62 | 5 mC at residues 3, 6, 9, and 16 |
| | | | pdU at residues 1, 8, 12, 15, 17, and 18 |
| | Probe | 9 | HEX/BHQ1 |
| | | | Reverse polarity C |
| V. cholerae | Primer | 56 | |
| | Primer | 11 | 5 mC at residues 8, 10, and 17 |
| | | | pdU at residues 3, 4, 5, 7, 11, 14, 16, |
| | | | 19, and 20 |
| | Probe | 12 | 5 mC at residue 20 |
| | | | pdU at residues 7, 13, 15, 16, and 18 |
| | | | HEX/BHQ1 |
| | | | Reverse polarity C |
| STEC O157 | Primer | 70 | 5 mC at residues 3, 6, 8, 12, and 15 |
| | Primer | 16 | 5 mC at residue 16 |
| | | | pdU at residues 4, 6, 7, 9, 11, 13, 15, |
| | | | 19, 20, 21, and 23 |
| | Probe | 73 | 5 mC at residues 3, 6, 13, 14, 19, and 22 |
| | | | pdU at residues 2, 4, 7, 8, 9, 10, 18, 18, |
| | | | and 20 |
| | | | CalRed610/BHQ2 |
| P. shigelloides | Primer | 2 | |
| | Primer | 49 | |
| | Probe | 48 | 5 mC at residues 3, 7, 8, 12, and 21 |
| | | | pdU at residues 5, 6, 9, 14, and 18 |
| | | | Quasar670/BHQ2 |

An internal control (IC) primer and probe set was also used (primers of SEQ ID NOs:69 and 39 and probe of SEQ ID NO: 14 labeled with Quasar 705 and BHQ2).

Results of Experiments A-D are shown in Tables 35-38, respectively.

TABLE 35
Yersinia Oligo Screening Results (Experiment A)

| Target Conc | Oligo | | | |
|---|---|---|---|---|
| (CFU/mL) | Set | Positivity | Avg Ct | Avg RFU |
| 10 | Nom | 2 | 38.52 | 9053 |
| | Set 1 | 3 | 37.95 | 15209 |
| | Set 2 | 3 | 36.90 | 21222 |
| | Set 3 | 2 | 42.68 | 1694 |
| | Set 5 | 1 | 39.79 | 6026 |
| 30 | Nom | 3 | 36.83 | 14723 |
| | Set 1 | 3 | 36.08 | 22226 |
| | Set 2 | 3 | 35.75 | 27132 |
| | Set 3 | 3 | 41.93 | 2288 |
| | Set 5 | 3 | 38.94 | 6404 |
| 70 | Nom | 3 | 36.69 | 15364 |
| | Set 1 | 3 | 35.50 | 23608 |
| | Set 2 | 3 | 35.21 | 31172 |
| | Set 3 | 3 | 40.11 | 3631 |
| | Set 5 | 3 | 38.04 | 7785 |

TABLE 36
Yersinia Inclusivity Results (Experiment B)

| | Positivity | | Avg Ct | | Avg RFU | |
|---|---|---|---|---|---|---|
| Sample ID | Set 1 | Set 2 | Set 1 | Set 2 | Set 1 | Set 2 |
| GP2387 | 1 | 1 | 30.25 | 29.75 | 38132 | 43004 |
| GP2388 | 1 | 1 | 30.07 | 30.82 | 35142 | 40311 |
| GP2398 | 1 | 1 | 29.40 | 30.09 | 37203 | 42157 |
| GP2399 | 1 | 1 | 27.99 | 27.54 | 36729 | 51091 |
| GP2441 | 1 | 1 | 29.60 | 29.88 | 35684 | 44047 |
| GP2430 | 1 | 1 | 30.03 | 30.01 | 34290 | 41611 |
| GP2431 | 1 | 1 | 30.14 | 30.25 | 33798 | 39681 |
| GP2432 | 1 | 1 | 29.19 | 30.22 | 35521 | 40416 |
| GP2433 | 1 | 1 | 29.68 | 29.98 | 38841 | 45832 |
| GP2436 | 1 | 1 | 29.36 | 29.26 | 35289 | 42935 |
| GP2498 | 1 | 1 | 33.75 | 34.05 | 27512 | 30013 |
| NY Bio 199 | 1 | 1 | 29.10 | 28.90 | 40593 | 54603 |
| NY Bio 204 | 1 | 1 | 32.15 | 31.29 | 36110 | 45720 |

TABLE 37
Multiplex 1E3 CFU/mL Results (Experiment C)

| | | | | | IC | | |
|---|---|---|---|---|---|---|---|
| | Oligo | Target Channel | | | Count of | | |
| Sample | Set | Positivity | Avg Ct | Avg RFU | Ct | Avg Ct | Avg RFU |
| Negative Control | 1 | — | — | — | 3/3 | 25.65 | 23538 |
| | 2 | — | — | — | 3/3 | 25.50 | 22690 |
| Y. enterocolitica | 1 | 3/3 | 30.57 | 38006 | 3/3 | 25.84 | 23151 |
| | 2 | 3/3 | 30.24 | 38569 | 3/3 | 25.54 | 23053 |
| V. cholerae | 1 | 3/3 | 31.16 | 18346 | 3/3 | 26.04 | 22335 |
| | 2 | 3/3 | 30.44 | 20728 | 3/3 | 25.70 | 21864 |
| V. | 1 | 3/3 | 29.70 | 22983 | 3/3 | 25.69 | 22407 |
| parahaemolyticus | 2 | 3/3 | 29.48 | 23235 | 3/3 | 25.89 | 21608 |
| V. vulnificus | 1 | 3/3 | 30.98 | 21270 | 3/3 | 25.79 | 23085 |
| | 2 | 3/3 | 31.50 | 20052 | 3/3 | 26.06 | 22177 |
| STEC O157 | 1 | 3/3 | 24.78 | 73943 | 3/3 | 25.88 | 22596 |
| | 2 | 3/3 | 25.12 | 79286 | 3/3 | 25.90 | 23248 |
| P. shigelloides | 1 | 2/2 | 30.41 | 40658 | 2/2 | 25.52 | 24903 |
| | 2 | 3/3 | 30.59 | 41586 | 3/3 | 25.50 | 24935 |

TABLE 38
Multiplex LoD Results (Experiment D)

| | Target Analyte | | | | | | IC | | | | | |
|---|---|---|---|---|---|---|---|---|---|---|---|---|
| | Positivity | | Avg Ct | | Avg RFU | | Positivity | | Avg Ct | | Avg RFU | |
| | Set | Set | Set | Set | Set | Set | Set | Set | Set | Set | Set | Set |
| Sample | 1 | 2 | 1 | 2 | 1 | 2 | 1 | 2 | 1 | 2 | 1 | 2 |
| Negative | | | | | | | 2/2 | 2/2 | 25.20 | 25.37 | 20643 | 19205 |
| Control | | | | | | | | | | | | |
| Y. ent 3 | 1/3 | | 38.30 | | 14044 | | 3/3 | 3/3 | 25.80 | 25.86 | 19687 | 19408 |
| Y. ent 10 | 2/3 | 3/3 | 36.48 | 38.07 | 21411 | 16180 | 3/3 | 3/3 | 26.10 | 26.42 | 19774 | 18453 |
| Y. ent 30 | 3/3 | 3/3 | 35.74 | 35.93 | 22579 | 23188 | 3/3 | 3/3 | 25.70 | 25.78 | 19908 | 18979 |
| Y. ent 70 | 3/3 | 3/3 | 34.68 | 34.13 | 24823 | 29998 | 3/3 | 3/3 | 25.96 | 25.72 | 19866 | 20231 |
| Y. ent 100 | 3/3 | 3/3 | 33.84 | 33.98 | 30460 | 30800 | 3/3 | 3/3 | 25.67 | 25.63 | 19646 | 20316 |
| Y. ent 300 | 3/3 | 3/3 | 32.48 | 32.61 | 31976 | 32318 | 3/3 | 3/3 | 25.75 | 26.05 | 19562 | 19385 |
| V. chol 30 | 3/3 | 3/3 | 35.89 | 36.26 | 12210 | 11452 | 3/3 | 3/3 | 25.79 | 26.02 | 20180 | 18381 |
| V. chol 90 | 3/3 | 3/3 | 34.50 | 35.06 | 14173 | 14066 | 3/3 | 3/3 | 25.95 | 26.41 | 19214 | 18214 |
| V. para 70 | 3/3 | 3/3 | 33.13 | 33.58 | 19358 | 17845 | 3/3 | 3/3 | 25.40 | 25.96 | 20206 | 19029 |
| V. para 210 | 3/3 | 3/3 | 31.31 | 32.05 | 21493 | 19611 | 3/3 | 3/3 | 25.36 | 25.98 | 20202 | 19377 |
| V. vul 300 | 3/3 | 3/3 | 32.87 | 33.21 | 19487 | 18895 | 3/3 | 3/3 | 25.81 | 25.75 | 19370 | 18897 |
| V. vul 900 | 3/3 | 3/3 | 31.17 | 31.12 | 21248 | 21183 | 3/3 | 3/3 | 25.68 | 25.66 | 19182 | 18777 |
| O157 30 | 3/3 | 3/3 | 36.99 | 36.47 | 29848 | 35172 | 3/3 | 3/3 | 26.01 | 26.09 | 19461 | 19195 |
| O157 90 | 3/3 | 3/3 | 35.90 | 36.49 | 48121 | 35779 | 3/3 | 3/3 | 25.72 | 25.73 | 19635 | 19518 |
| P. shig 7 | 3/3 | 3/3 | 33.73 | 34.25 | 35344 | 33906 | 3/3 | 3/3 | 25.62 | 25.89 | 21254 | 19927 |
| P. shig 210 | 3/3 | 3/3 | 32.40 | 32.82 | 37898 | 36507 | 3/3 | 3/3 | 25.60 | 26.09 | 20915 | 19847 |

Based on the oligo screening results in Experiment A, Yersinia oligo sets 1 and 2 showed promising results given the high amplification curve and capability to detect 100% at 10 CFU/mL. These oligo sets were selected for further evaluation in Experiment.

In Experiment B, set 1 and set 2 were successful in detecting all inclusivity strains and previously negative (by YstA system) clinical samples, so both sets were moved forward for multiplex system testing.

Experiments C and D proved both multiplex sets are capable of detecting all bacterial targets, but Set 1 had better detection at the lower Y. enterocolitica concentration tested in Experiment D. Set 1 also showed better Ct values and RFU ranges.

### Example 14: Analytical Sensitivity

The primers and probes shown in Table 39 were evaluated in multiplex for analytical sensitivity in Cary-Blair Stool matrix using a representative strain for each species (Y. enterocolitica, V parahaemolyticus, V. vulnificus, V. cholerae, STEC O157, and P. shigelloides).

TABLE 39
Multiplex Primer and Probe Set for GI Bacteria

| | Oligo | | |
|---|---|---|---|
| Target | Type | SEQ ID NO | Modifications |
| Y. enterocolitica | Primer | 26 | |
| | Primer | 46 | 5 mC at residues 13 and 18 |
| | Probe | 40 | 5 mC at residues 7, 8, 9, 10, and 18 |
| | | | FAM/BHQ1 |
| | | | Reverse polarity C |
| V. | Primer | 51 | |
| parahaemolyticus | Primer | 76 | pdU at residues 2 and 10 |
| | Probe | 33 | CalOrange560/BHQ1 |
| | | | Reverse polarity C |
| V. vulnificus | Primer | 66 | pdU at residues 6 and 10 |
| | Primer | 62 | 5 mC at residues 3, 6, 9, and 16 |
| | | | pdU at residues 1, 8, 12, 15, 17, and 18 |
| | Probe | 9 | CalOrange560/BHQ1 |
| | | | Reverse polarity C |
| V. cholerae | Primer | 56 | |
| | Primer | 11 | 5 mC at residues 8, 10, and 17 |
| | | | pdU at residues 3, 4, 5, 7, 11, 14, 16, |
| | | | 19, and 20 |
| | Probe | 12 | 5 mC at residue 20 |
| | | | pdU at residues 7, 13, 15, 16, and 18 |
| | | | CalOrange560/BHQ1 |
| | | | Reverse polarity C |
| STEC O157 | Primer | 70 | 5 mC at residues 3, 6, 8, 12, and 15 |
| | Primer | 16 | 5 mC at residue 16 |
| | | | pdU at residues 4, 6, 7, 9, 11, 13, 15, |
| | | | 19, 20, 21, and 23 |
| | Probe | 73 | 5 mC at residues 3, 6, 13, 14, 19, and 22 |
| | | | pdU at residues 2, 4, 7, 8, 9, 10, 17, 18, |
| | | | and 20 |
| | | | CalRed610/BHQ2 |
| P. shigelloides | Primer | 2 | |
| | Primer | 49 | |
| | Probe | 48 | 5 mC at residues 3, 7, 8, 12, and 21 |
| | | | pdU at residues 5, 6, 9, 14, and 18 |
| | | | Quasar670/BHQ2 |

Cary-Blair Stool (CBS) matrix was prepared by mixing negative stool samples collected in Cary-Blair media, diluted with negative STM at 1:20 fold-dilution before testing. LoD confirmation was performed for each target with the lowest concentration ≥95% positivity at least 10 replicates in Cary-Blair Stool (CBS) matrix diluted in STM. The estimated LoD is the lowest concentration to obtain ≥95% positivity in the confirmation run. Table 40 lists the summary LOD data for all targets.

TABLE 40
Summary of LOD Determination Results

| | | | | | | | | Slope at |
|---|---|---|---|---|---|---|---|---|
| | Conc | N | Ct | Ct | RFU | RFU | Bckgrnd | Threshold |
| Target | (CFU/mL) | Positive | Avg | StdDev | Avg | StdDev | RFU Avg | Avg |
| Yersinia | 10 | 3/5 | 35.91 | 0.15 | 18596 | 1427 | 5387 | 523 |
| enterocolitica | 30 | 4/5 | 35.59 | 0.55 | 20331 | 2244 | 5459 | 483 |
| | 100 | 5/5 | 33.65 | 1.03 | 29306 | 4813 | 5335 | 575 |
| Vibrio | 10 | 17/20 | 36.69 | 0.80 | 7216 | 1757 | 5682 | 257 |
| parahaemolyticus | 30 | 5/5 | 36.09 | 1.56 | 9131 | 3387 | 5924 | 313 |
| | 100 | 19/20 | 33.37 | 0.82 | 14518 | 1524 | 5543 | 313 |
| Vibrio cholerae | 3 | 2/5 | 37.87 | 0.01 | 7474 | 251 | 5690 | 226 |
| | 10 | 5/5 | 36.32 | 0.88 | 10729 | 1989 | 5364 | 298 |
| | 30 | 5/5 | 35.03 | 0.97 | 13334 | 2005 | 5562 | 317 |
| | 70 | 5/5 | 33.38 | 0.75 | 16247 | 1098 | 5451 | 278 |
| | 100 | 5/5 | 33.23 | 0.92 | 15318 | 1138 | 5609 | 290 |
| | 300 | 5/5 | 31.42 | 0.56 | 18321 | 864 | 5805 | 317 |
| | 500 | 5/5 | 30.79 | 0.48 | 19234 | 460 | 5396 | 332 |
| Vibrio vulnificus | 3 | 1/5 | 41.58 | — | 1012 | — | 5549 | 122 |
| | 10 | 5/5 | 37.48 | 0.67 | 2781 | 435 | 5547 | 176 |
| | 30 | 5/5 | 36.75 | 1.01 | 3835 | 1281 | 5576 | 186 |
| | 70 | 5/5 | 34.16 | 0.61 | 8812 | 1581 | 5829 | 270 |
| | 100 | 5/5 | 33.89 | 0.38 | 7523 | 716 | 5110 | 228 |
| | 300 | 5/5 | 32.49 | 0.52 | 10593 | 895 | 5329 | 276 |
| | 500 | 5/5 | 31.26 | 0.16 | 13236 | 841 | 5485 | 317 |
| STEC-O157 | 10 | 3/5 | 39.25 | 0.65 | 6923 | 1914 | 930 | 516 |
| | 30 | 5/5 | 35.39 | 0.79 | 20852 | 5950 | 897 | 561 |
| | 100 | 5/5 | 33.48 | 0.58 | 34836 | 3838 | 887 | 642 |
| Plesiomonas | 3 | 0/5 | — | — | — | — | 569 | — |
| shigelloides | 10 | 4/5 | 39.01 | 1.94 | 3785 | 1435 | 581 | 445 |
| | 30 | 19/20 | 36.15 | 0.83 | 7530 | 2548 | 630 | 467 |

The LoD for assay targets were confirmed in Cary-Blair Stool (CBS) matrix. Table 41 shows the LoD confirmation data. Yersinia enterocolitica, STEC-O157, Plesiomonas shigelloides, Vibrio parahaemolyticus, and Vibrio cholerae passed at 100% positivity at 1×LoD. Vibrio vulnificus passed at 100% positivity at 7×LoD instead of 1×LoD. Therefore, Vibrio vulnificus confirmed LoD is 70 cells/mL instead of 10 cells/mL.

TABLE 41
LoD Confirmation Results

| | | | | | | | | Slope at |
|---|---|---|---|---|---|---|---|---|
| | Conc | N | Percent | | Ct | RFU | RFU | Threshold |
| Target | (CFU/mL) | Positive | Positive | Ct Avg | StdDev | Avg | StdDev | Avg |
| Yersinia | 100 | 10/10 | 100% | 32.08 | 0.26 | 34915 | 1906 | 584 |
| enterocolitica | | | | | | | | |
| Vibrio | 30 | 10/10 | 100% | 35.19 | 0.95 | 10628 | 1751 | 273 |
| parahaemolyticus | | | | | | | | |
| Vibrio cholerae | 10 | 10/10 | 100% | 36.05 | 0.54 | 11296 | 1336 | 288 |
| Vibrio vulnificus | 70 | 10/10 | 100% | 34.42 | 0.74 | 7587 | 1317 | 247 |
| | 30 | 17/20 | 85% | 36.83 | 1.19 | 3877 | 1491 | 188 |
| | 10 | 16/20 | 80% | 39.15 | 1.92 | 1939 | 1089 | 147 |
| STEC-O157 | 30 | 10/10 | 100% | 34.13 | 0.59 | 27134 | 4413 | 593 |
| Plesiomonas | 30 | 10/10 | 100% | 36.06 | 0.62 | 7892 | 1962 | 499 |
| shigelloides | | | | | | | | |

The final confirmed LoD for each assay target is shown in Table 42.

TABLE 42
Confirmed LoD

| | Concentration |
|---|---|
| | (CFU/mL) in |
| Target | lysis tube |
| Yersinia | 30 |
| V. | 70 |
| parahaemolyticus | |
| V. cholerae | 30 |
| V. vulnificus | 300 |
| STEC O157 | 300 |
| Plesiomonas | 70 |

### LoD Comparison

The GI bacteria multiplex assay (“Assay”) LoDs in Cary-Blair Stool Matrix (CBS) were comparable to commercially available assays such as BioFire FilmArray GI Panel and BD MAX Extended Enteric Bacteria Panel. See LoD comparison in Table 43 (BioFire and BD MAX data from published Package Inserts). Concentrations are presented as the final concentration in the lysis/sample tube after dilution into STM and as the original concentration as stool in Cary-Blair Stool Matrix (CBS). Dilution factor of 20 was calculated when ~150 μL CBS sample was added to 2.85 mL STM lysis buffer for a total of 3 mL. The GI bacteria multiplex assay described herein has comparable or lower Limit of Detection (LoD) to other commercial assays/tests for relevant bacterial targets.

TABLE 43
LoD Comparison
LOD (CFU/mL)

| | | Assay | | | |
|---|---|---|---|---|---|
| | | in | | | BD |
| | | Lysis | Assay | | MAX |
| | | Tube | in | BioFire | in |
| Channel | Target | (STM) | CBS* | in CBS | CBS |
| FAM | Yersinia enterocolitica | 100 | 2,000 | 50,000 | 22,723 |
| HEX | Vibrio parahaemolyticus | 30 | 600 | 80,000 | 12,424 |
| | Vibrio vulnificus | 70 | 1,400 | — | 13,093 |
| | Vibrio cholerae | 10 | 200 | 8,000 | 25,238 |
| ROX | STEC O157 | 30 | 600 | 10,000 | — |
| Q670 | Plesiomonas | 30 | 600 | 1,000 | 25,712 |
| | shigelloides | | | | |

*Representation of Assay-established LoD with the applied 20-fold dilution factor

### SEQUENCES

TABLE 44
Exemplary Oligonucleotide Sequences

| SEQ | | | Target | |
|---|---|---|---|---|
| ID NO | Sequence (5′ → 3′) | Target species | gene | Function |
| 1 | AAAATGCGGTTGCCACTTAAACCCC | P. shigelloides | hugA | amplification oligomer |
| 2 | AACAACCGGCCAGCGTGG | P. shigelloides | hugA | amplification oligomer |
| 3 | AACACTTTATCGCTACTGTCATTAGGT | V. cholerae | ompW | amplification oligomer |
| 4 | AACCCTGTTTCACCATCATAAAGGTCGC | Y. enterocolitica | ail | amplification oligomer |
| 5 | AAGATTATCGACGCCGCACG | V. vulnificus | gyrB | amplification oligomer |
| 6 | AAGCATTTTATCAATCAGGTTCATTCCG | Y. enterocolitica | ail | amplification oligomer |
| 7 | ACATCCATGTGATATGGAACAAATTG | STEC O157 | rfbE | detection probe |
| 8 | ACCGGCTGAGCCTCAGACTCAGC | P. shigelloides | hugA | detection probe |
| 9 | ACGACTTCTTAGCCGAGCACCCAAGCG | V. vulnificus | gyrB | detection probe |
| 10 | ACGGTTTTATGGTTAAGACTCCCG | Y. enterocolitica | ail | amplification oligomer |
| 11 | ACTTTATCGCTACTGTCATTAGG | V. cholerae | ompW | amplification oligomer |
| 12 | AGAAGGTGACTTTATTGTGCGCG | V. cholerae | ompW | detection probe |
| 13 | AGCGCACCATTACTGGTGGTTA | Y. enterocolitica | invA | amplification oligomer |
| 14 | AGCGGAATCACAAGTCAATCATCGCGC | Internal Control | — | detection probe |
| 15 | AGCGGTTGAATCGGCGATGGGTGA | V. parahaemolyticus | gyrB | detection probe |
| 16 | AGCTATTATGTGTGTCCATTTATAC | STEC O157 | rfbE | amplification oligomer |
| 17 | AGGCACAAATTCAGCAACAACAAC | P. shigelloides | hugA | amplification oligomer |
| 18 | AGGGCAGTTCAGTGATGCATTATCGAC | Y. enterocolitica | ystA | detection probe |
| 19 | ATAAGCAGTCGATTCCCCAAG | V. cholerae | toxR | amplification oligomer |
| 20 | ATCTTCGCTTCGCTTGGGT | V. parahaemolyticus/ | gyrB | amplification oligomer |
| | | V. vulnificus | | |
| 21 | ATCTTCGCTTCGCTTGGGTT | V. parahaemolyticus/ | gyrB | amplification oligomer |
| | | V. vulnificus | | |
| 22 | ATGATGGTGCGCTTGCTG | Y. enterocolitica | invA | amplification oligomer |
| 23 | ATGGTCAATTAGAGACAAAG | Internal Control | — | amplification oligomer |
| 24 | ATGTGACCGAGTTCAACGTCAGGG | Y. enterocolitica | ail | amplification oligomer |
| 25 | CAAGAGAACTTTCTTTGAGGGAGAATTT | Y. enterocolitica | invA | detection probe |
| 26 | CACGGCAAATCGTACATCA | Y. enterocolitica | invA | amplification oligomer |
| 27 | CCAACAAAGTTGGATGTATGGCTT | Y. enterocolitica | invA | detection probe |
| 28 | CCACGCCAATACGATGATTGT | Y. enterocolitica | invA | amplification oligomer |
| 29 | CCCATCACGGTGAAATTTACAGTGAC | Y. enterocolitica | invA | detection probe |
| 30 | CCCTTAATATCGGCGCTGG | Y. enterocolitica | invA | amplification oligomer |
| 31 | CCGCAGTGGAATCCTTCATGGCAGACA | V. vulnificus | gyrB | detection probe |
| 32 | CCGTACTTGCAGCCCTAAGC | V. cholerae | ompW | amplification oligomer |
| 33 | CCTCTAGCGGTTGTGGGTGATACGG | V. parahaemolyticus | gyrB | detection probe |
| 34 | CGAAATCACGAGATTGATGCCAACCAT | Y. enterocolitica | invA | detection probe |
| 35 | CGCGCGGGTATTGCCTCGGT | V. cholerae | ompW | detection probe |
| 36 | CGGAATCACAAGTCAATCATCGCGCA | Internal Control | — | detection probe |
| 37 | CGTGCGGCGTCGATAATCTT | V. vulnificus | gyrB | amplification oligomer |
| 38 | CGTTCACTATTGGTCTCTGC | Internal Control | — | amplification oligomer |
| 39 | CGTTCACTATTGGTCTCTGCA | Internal Control | — | amplification oligomer |
| 40 | CTGTTGCCCCTGAATATCTAAAGTGAC | Y. enterocolitica | invA | detection probe |
| 41 | GAAGCCTGGACTGATTATTTACA | Y. enterocolitica | invA | amplification oligomer |
| 42 | GCAGTGGAATCCTTCATGGC | V. vulnificus | gyrB | amplification oligomer |
| 43 | GCCAAACCAAAGACAAACTGGT | V. vulnificus | gyrB | amplification oligomer |
| 44 | GCTATTATGTGTGTCCATTTATACG | STEC O157 | rfbE | amplification oligomer |
| 45 | GGCAAATCAGGAAGTAAAACACTGG | Y. enterocolitica | invA | amplification oligomer |
| 46 | GGGTGTGATAAGCATTGCATTAAC | Y. enterocolitica | invA | amplification oligomer |
| 47 | GTGCGGCGTCGATAATCTTAG | V. vulnificus | gyrB | amplification oligomer |
| 48 | TACATTCCTAACGTGGATGTCAG | P. shigelloides | hugA | detection probe |
| 49 | TACTGACGGGCTTTGCGCATTAG | P. shigelloides | hugA | amplification oligomer |
| 50 | TCATTGCGTAGGGCCAGCGTTATTCTGC | Y. enterocolitica | ail | detection probe |
| 51 | TCCACACGCAAACTTACC | V. parahaemolyticus | gyrB | amplification oligomer |
| 52 | TCCACACGCAAACTTACCG | V. parahaemolyticus | gyrB | amplification oligomer |
| 53 | TCCACACGCAAACTTACCGT | V. parahaemolyticus | gyrB | amplification oligomer |
| 54 | TCCCAATCACTACTGACTTCGGCTGG | Y. enterocolitica | ystA | amplification oligomer |
| 55 | TCCGCAGTGGAATCCTTCAT | V. vulnificus | gyrB | amplification oligomer |
| 56 | TCCGCTCCTGTATTTGCTCA | V. cholerae | ompW | amplification oligomer |
| 57 | TCCGCTCCTGTATTTGCTCAC | V. cholerae | ompW | amplification oligomer |
| 58 | TCGAGCCAAACCAAAGACAAAC | V. parahaemolyticus/ | gyrB | amplification oligomer |
| | | V. vulnificus | | |
| 59 | TCTACTTTTGGCCAGTTCTAC | STEC O157 | rfbE | detection probe |
| 60 | TGAAGCAGAGTCATACCAAGG | Y. enterocolitica | invA | amplification oligomer |
| 61 | TGACGGGCTTTGCGCATTAGCGC | P. shigelloides | hugA | detection probe |
| 62 | TGCGGCGTCGATAATCTTAG | V. vulnificus | gyrB | amplification oligomer |
| 63 | TGCGTATCCATCTGCCAGGCAACGACTT | Y. enterocolitica | ail | detection probe |
| | C | | | |
| 64 | TGCTATTAACTGCCAACTCACTTTGA | V. cholerae | ompW | amplification oligomer |
| 65 | TGCTGTCTTCATTTGGAGCATTC | Y. enterocolitica | ystA | amplification oligomer |
| 66 | TGGAATCCTTCATGGCAGAC | V. vulnificus | gyrB | amplification oligomer |
| 67 | TGGAGCCGATTTATTCGCCACGA | V. cholerae | toxR | detection probe |
| 68 | TGGCCGGTTGTTGTTGCTGAATTTG | P. shigelloides | hugA | amplification oligomer |
| 69 | TGGGACATGGTCAATTAGAGACA | Internal Control | — | amplification oligomer |
| 70 | TTCAGCGCAATCTTCAATTAC | STEC O157 | rfbE | amplification oligomer |
| 71 | TTCAGCGCAATCTTCAATTACAAA | STEC O157 | rfbE | amplification oligomer |
| 72 | TTCCTAACGTGGATGTCAGTGGCGGTCC | P. shigelloides | hugA | detection probe |
| 73 | TTCTACTTTTGGCCAGTTCTAC | STEC O157 | rfbE | detection probe |
| 74 | TTCTACTTTTGGCCAGTTCTACAAT | STEC O157 | rfbE | detection probe |
| 75 | TTCTGCACTTGGCCAGAAAC | V. parahaemolyticus | gyrB | amplification oligomer |
| 76 | TTCTGCACTTGGCCAGAAACGA | V. parahaemolyticus | gyrB | amplification oligomer |
| 77 | TTGCCGGGCGCTCATTATA | Y. enterocolitica | invA | amplification oligomer |
| 78 | TTGGGTGCTCGGCTAAGAAG | V. vulnificus | gyrB | amplification oligomer |
| 79 | TTTCATTTGCCGCCAACGC | P. shigelloides | hugA | amplification oligomer |
| 80 | TTCCTAACGTGGATGTCAGTGGCGGTCC | P. shigelloides | hugA | detection probe |
| | GC | | | |
| 81 | TGGCCGGTTGTTGTTGCTGAATTTGTG | P. shigelloides | hugA | amplification oligomer |
| 82 | TGGAGCCGATTTATTCGCCACGAC | V. cholera | toxR | detection probe |
| 83 | TGCGTATCCATCTGCCAGGCAACGACTT | Y. enterocolitica | ail | detection probe |
| | CGC | | | |
| 84 | GTGCGGCGTCGATAATCTT | V. vulnificus | gyrB | amplification oligomer |
| 85 | GTACTTGCAGCCCTAAGC | V. cholerae | ompW | amplification oligomer |
| 86 | GCTTCGCTTGGGTTTTCGAC | V. parahaemolyticus | gyrB | amplification oligomer |
| 87 | GCTATTATGTGTGTCCATTTATAC | STEC O157 | rfbE | amplification oligomer |
| 88 | GCGCGCGGGTATTGCCTCGGT | V. cholerae | ompW | detection probe |
| 89 | GCCTCTAGCGGTTGTGGGTGATACGG | V. parahaemolyticus | gyrB | detection probe |
| 90 | CTTGCTCATTCSATAGGCTGG | STEC O157 | rfbE | amplification oligomer |
| 91 | CTCTAGCGGTTGTGGGTGATACGG | V. parahaemolyticus | gyrB | detection probe |
| 92 | CGTACTTGCAGCCCTAAGC | V. cholerae | ompW | amplification oligomer |
| 93 | CCGAGTACATTGGCATCGTG | STEC O157 | rfbE | amplification oligomer |
| 94 | ATACGCAGAGTCAAGCAGTG | V. cholerae | toxR | amplification oligomer |
| 95 | AGGCACAAATTCAGCAACAACAACCGG | P. shigelloides | hugA | amplification oligomer |
| 96 | ACTAGGTAGATTAATTCCACGCCAACC | STEC O157 | rfbE | detection probe |
| 97 | ACGGTTTTATGGTTAAGACTCCCGC | Y. enterocolitica | ail | amplification oligomer |
| 98 | ACCTGCAAAATGCGGTTGCCAC | P. shigelloides | hugA | amplification oligomer |
| 99 | ACAGGGTAAAAAACTGGCCTTGTTTCG | STEC O157 | rfbE | detection probe |
| 100 | ACACGATGCCAATGTACTCGG | STEC O157 | rfbE | amplification oligomer |
| 101 | AATTCTAACTAGGACCGCAGAGG | STEC O157 | rfbE | amplification oligomer |
| 102 | AAGCATTTTATCAATCAGGTTCATTCCG | Y. enterocolitica | ail | amplification oligomer |
| | G | | | |
| 103 | AAGCACCCTATAGCTGAGGATCTTGGTT | STEC O157 | rfbE | detection probe |
| 104 | AAGAGAGGAATTAAGGAATCACCTTGC | STEC O157 | rfbE | detection probe |
| 105 | AACACTTTATCGCTACTGTCATTAGG | V. cholerae | ompW | amplification oligomer |
| 106 | AAATGCGGTTGCCACTTAAACC | P. shigelloides | hugA | amplification oligomer |
| 107 | CTATTATGTGTGTCCATTTATACG | STEC O157 | rfbE | amplification oligomer |
| 121 | CCTCTAGCGGTTGTGGGTGATACGG | V. parahaemolyticus | gyrB | detection probe |

From the foregoing, it will be appreciated that, although specific embodiments have been described herein for purposes of illustration, various modifications may be made without deviating from the spirit and scope of the disclosure. All publications, patents, and patent applications cited herein are hereby incorporated by reference in their entireties for all purposes.

1. (canceled)

2. A composition or kit for determining the presence or absence of at least one enteric pathogen in a sample, wherein the at least one enteric pathogen is selected from the group consisting of Yersinia enterocolitica, Vibrio spp., Shiga Toxin Expressing E. coli (STEC) O157, and Plesiomonas shigelloides, said composition or kit comprising a set of oligonucleotides comprising at least one of (a)-(d):
(a) a Yersinia-specific amplification oligomer set capable of amplifying a target region of a Yersinia enterocolitica target nucleic acid, wherein the Yersinia-specific amplification oligomer set comprises first and second Yersinia-specific amplification oligomers respectively comprising target-hybridizing sequences as shown in (i) SEQ ID NO:26 and SEQ ID NO:46, including from 0 to 16 nucleotide analogs, (ii) SEQ ID NO:97 and SEQ ID NO:4, including from 0 to 16 nucleotide analogs, (iii) SEQ ID NO: 102 and SEQ ID NO:24, including from 0 to 16 nucleotide analogs, (iv) SEQ ID NO:6 and SEQ ID NO:24, including from 0 to 16 nucleotide analogs, (v) SEQ ID NO: 13 and SEQ ID NO:22, including from 0 to 16 nucleotide analogs, (vi) SEQ ID NO:77 and SEQ ID NO:22, including from 0 to 16 nucleotide analogs, (vii) SEQ ID NO:77 and SEQ ID NO:41, including from 0 to 16 nucleotide analogs, (viii) SEQ ID NO:28 and SEQ ID NO:30, including from 0 to 16 nucleotide analogs, (ix) SEQ ID NO:60 and SEQ ID NO:45, including from 0 to 16 nucleotide analogs, (x) SEQ ID NO:65 and SEQ ID NO:54, including from 0 to 16 nucleotide analogs, or (xi) SEQ ID NO: 10 and SEQ ID NO:4, including from 0 to 16 nucleotide analogs;
(b) a Vibrio-specific amplification oligomer set capable of amplifying a target region of a Vibrio spp. target nucleic acid, wherein the Vibrio spp. is one or more of V. parahaemolyticus, V. vulnificus, and V. cholerae, and wherein the Vibrio-specific amplification oligomer set comprises
(b-1) first and second V. parahaemolyticus-specific amplification oligomers respectively comprising target-hybridizing sequences as shown in (i) SEQ ID NO:51 and SEQ ID NO:76, including from 0 to 16 nucleotide analogs, (ii) SEQ ID NO:58 and SEQ ID NO:21, including from 0 to 16 nucleotide analogs, (iii) SEQ ID NO:58 and SEQ ID NO: 86, including from 0 to 16 nucleotide analogs, (iv) SEQ ID NO:53 and SEQ ID NO: 75, including from 0 to 16 nucleotide analogs, (v) SEQ ID NO:58 and SEQ ID NO:20, including from 0 to 16 nucleotide analogs, (vi) SEQ ID NO:52 and SEQ ID NO:75, including from 0 to 16 nucleotide analogs, (vii) SEQ ID NO:51 and SEQ ID NO:75, including from 0 to 16 nucleotide analogs, (viii) SEQ ID NO:52 and SEQ ID NO:76, including from 0 to 16 nucleotide analogs, or (ix) SEQ ID NO:53 and SEQ ID NO:76, including from 0 to 16 nucleotide analogs; and/or
(b-2) first and second V. vulnificus-specific amplification oligomers respectively comprising target-hybridizing sequences as shown in (i) SEQ ID NO:66 and SEQ ID NO:62, including from 0 to 16 nucleotide analogs, (ii) SEQ ID NO:58 and SEQ ID NO:21, including from 0 to 16 nucleotide analogs, (iii) SEQ ID NO:42 and SEQ ID NO:37, including from 0 to 16 nucleotide analogs, (iv) SEQ ID NO:55 and SEQ ID NO:37, including from 0 to 16 nucleotide analogs, (v) SEQ ID NO:43 and SEQ ID NO:78, including from 0 to 16 nucleotide analogs, (vi) SEQ ID NO:58 and SEQ ID NO:20, including from 0 to 16 nucleotide analogs, (vii) SEQ ID NO:42 and SEQ ID NO:84, including from 0 to 16 nucleotide analogs, (viii) SEQ ID NO:42 and SEQ ID NO:47, including from 0 to 16 nucleotide analogs, (ix) SEQ ID NO:55 and SEQ ID NO:84, including from 0 to 16 nucleotide analogs, (x) SEQ ID NO:55 and SEQ ID NO:47, including from 0 to 16 nucleotide analogs, or (xi) SEQ ID NO:55 and SEQ ID NO:5, including from 0 to 16 nucleotide analogs; and/or
(b-3) first and second V. cholerae-specific amplification oligomers respectively comprising target-hybridizing sequences as shown in (i) SEQ ID NO:56 and SEQ ID NO: 11, including from 0 to 16 nucleotide analogs, (ii) SEQ ID NO:32 and SEQ ID NO:3, including from 0 to 16 nucleotide analogs, (iii) SEQ ID NO:57 and SEQ ID NO:64, including from 0 to 16 nucleotide analogs, (iv) SEQ ID NO:94 and SEQ ID NO: 19, including from 0 to 16 nucleotide analogs, (v) SEQ ID NO:32 and SEQ ID NO: 105, including from 0 to 16 nucleotide analogs, (vi) SEQ ID NO:92 and SEQ ID NO: 3, including from 0 to 16 nucleotide analogs; (vii) SEQ ID NO:92 and SEQ ID NO: 105, including from 0 to 16 nucleotide analogs, (viii) SEQ ID NO:85 and SEQ ID NO:3, including from 0 to 16 nucleotide analogs, or (ix) SEQ ID NO:85 and SEQ ID NO: 105, including from 0 to 16 nucleotide analogs;
(c) a STEC-specific amplification oligomer set capable of amplifying a target region of a STEC O157 target nucleic acid, wherein the STEC-specific amplification oligomer set comprises first and second STEC-specific amplification oligomers respectively comprising target-hybridizing sequences as shown in (i) SEQ ID NO: 70 and SEQ ID NO: 16, including from 0 to 16 nucleotide analogs, (ii) SEQ ID NO:93 and SEQ ID NO:101, including from 0 to 16 nucleotide analogs, (iii) SEQ ID NO:71 and SEQ ID NO: 44, including from 0 to 16 nucleotide analogs, (iv) SEQ ID NO: 90 and SEQ ID NO: 100, including from 0 to 16 nucleotide analogs, (v) SEQ ID NO:71 and SEQ ID NO: 16, including from 0 to 16 nucleotide analogs, (vi) SEQ ID NO:70 and SEQ ID NO:44, including from 0 to 16 nucleotide analogs, (vii) SEQ ID NO:70 and SEQ ID NO: 107, including from 0 to 16 nucleotide analogs, (viii) SEQ ID NO:70 and SEQ ID NO:87, including from 0 to 16 nucleotide analogs, (ix) SEQ ID NO:71 and SEQ ID NO: 107, including from 0 to 16 nucleotide analogs, or (x) SEQ ID NO:71 and SEQ ID NO: 87, including from 0 to 16 nucleotide analogs; and
(d) a Plesiomonas-specific amplification oligomer set capable of amplifying a target region of a Plesiomonas shigelloides target nucleic acid, wherein the Plesiomonas-specific amplification oligomer set comprises first and second Plesiomonas-specific amplification oligomers respectively comprising target-hybridizing sequences as shown in (i) SEQ ID NO:2 and SEQ ID NO:49, including from 0 to 16 nucleotide analogs, (ii) SEQ ID NO:95 and SEQ ID NO: 1, including from 0 to 16 nucleotide analogs, (iii) SEQ ID NO:79 and SEQ ID NO:81, including from 0 to 16 nucleotide analogs, (iv) SEQ ID NO: 17 and SEQ ID NO: 1, including from 0 to 16 nucleotide analogs, (v) SEQ ID NO:95 and SEQ ID NO: 106, including from 0 to 16 nucleotide analogs, (vi) SEQ ID NO: 17 and SEQ ID NO: 106, including from 0 to 16 nucleotide analogs, (vii) SEQ ID NO:79 and SEQ ID NO:68, including from 0 to 16 nucleotide analogs, (viii) SEQ ID NO: 17 and SEQ ID NO:98, including from 0 to 16 nucleotide analogs, or (ix) SEQ ID NO:95 and SEQ ID NO:98, including from 0 to 16 nucleotide analogs.

3. The composition or kit of claim 2, wherein the set of oligonucleotides comprises the Yersinia-specific amplification oligomer set.

4. (canceled)

5. The composition or kit of claim 3, wherein the set of oligonucleotides further comprises a Yersinia-specific detection probe comprising a target-hybridizing sequence substantially corresponding to the nucleotide sequence of
SEQ ID NO:40 if the Yersinia-specific amplification oligomer set comprises first and second Yersinia-specific amplification oligomers respectively comprising target-hybridizing sequences as shown in SEQ ID NO:26 and SEQ ID NO:46, including from 0 to 16 nucleotide analogs;
SEQ ID NO:50 if the Yersinia-specific amplification oligomer set comprises first and second Yersinia-specific amplification oligomers respectively comprising target-hybridizing sequences as shown in (i) SEQ ID NO:97 and SEQ ID NO:4, including from 0 to 16 nucleotide analogs or (ii) SEQ ID NO: 10 and SEQ ID NO: 4, including from 0 to 16 nucleotide analogs;
SEQ ID NO:63 or SEQ ID NO:83 if the Yersinia-specific amplification oligomer set comprises first and second Yersinia-specific amplification oligomers respectively comprising target-hybridizing sequences as shown in (i) SEQ ID NO: 102 and SEQ ID NO:24, including from 0 to 16 nucleotide analogs or (ii) SEQ ID NO:6 and SEQ ID NO:24, including from 0 to 16 nucleotide analogs;
SEQ ID NO:29 if the Yersinia-specific amplification oligomer set comprises first and second Yersinia-specific amplification oligomers respectively comprising target-hybridizing sequences as shown in (i) SEQ ID NO: 13 and SEQ ID NO:22, including from 0 to 16 nucleotide analogs or (ii) SEQ ID NO:77 and SEQ ID NO:22, including from 0 to 16 nucleotide analogs;
SEQ ID NO:34 if the Yersinia-specific amplification oligomer set comprises first and second Yersinia-specific amplification oligomers respectively comprising target-hybridizing sequences as shown in SEQ ID NO:77 and SEQ ID NO:41, including from 0 to 16 nucleotide analogs;
SEQ ID NO:27 if the Yersinia-specific amplification oligomer set comprises first and second Yersinia-specific amplification oligomers respectively comprising target-hybridizing sequences as shown in SEQ ID NO:28 and SEQ ID NO:30, including from 0 to 16 nucleotide analogs;
SEQ ID NO:25 if the Yersinia-specific amplification oligomer set comprises first and second Yersinia-specific amplification oligomers respectively comprising target-hybridizing sequences as shown in SEQ ID NO:60 and SEQ ID NO:45, including from 0 to 16 nucleotide analogs; or
SEQ ID NO: 18 if the Yersinia-specific amplification oligomer set comprises first and second Yersinia-specific amplification oligomers respectively comprising target-hybridizing sequences as shown in SEQ ID NO:65 and SEQ ID NO:54, including from 0 to 16 nucleotide analogs.

6. (canceled)

7. (canceled)

8. The composition or kit of claim 2, wherein the set of oligonucleotides comprises the first and second V. parahaemolyticus-specific amplification oligomers.

9. (canceled)

10. The composition or kit of claim 8, wherein the set of oligonucleotides further comprises a V. parahaemolyticus-specific detection probe comprising a target-hybridizing sequence substantially corresponding to the nucleotide sequence of
SEQ ID NO:33, SEQ ID NO:89, SEQ ID NO:91, or SEQ ID NO: 121 if the V. parahaemolyticus-specific amplification oligomer set comprises first and second V. parahaemolyticus-specific amplification oligomers respectively comprising target-hybridizing sequences as shown in (i) SEQ ID NO:51 and SEQ ID NO:76, including from 0 to 16 nucleotide analogs, (ii) SEQ ID NO:53 and SEQ ID NO:75, including from 0 to 16 nucleotide analogs, (iii) SEQ ID NO:52 and SEQ ID NO:75, including from 0 to 16 nucleotide analogs, (iv) SEQ ID NO:51 and SEQ ID NO:75, including from 0 to 16 nucleotide analogs, (v) SEQ ID NO:52 and SEQ ID NO:76, including from 0 to 16 nucleotide analogs, or (vi) SEQ ID NO:53 and SEQ ID NO:76, including from 0 to 16 nucleotide analogs; or
SEQ ID NO: 15 if the V. parahaemolyticus-specific amplification oligomer set comprises first and second V. parahaemolyticus-specific amplification oligomers respectively comprising target-hybridizing sequences as shown in (i) SEQ ID NO:58 and SEQ ID NO:21, including from 0 to 16 nucleotide analogs, (ii) SEQ ID NO:58 and SEQ ID NO:86, including from 0 to 16 nucleotide analogs, or (iii) SEQ ID NO:58 and SEQ ID NO:20, including from 0 to 16 nucleotide analogs.

11. (canceled)

12. The composition or kit of claim 2, wherein the set of oligonucleotides comprises the first and second V. vulnificus-specific amplification oligomers.

13. (canceled)

14. The composition or kit of claim 12, wherein the set of oligonucleotides further comprises a V. vulnificus-specific detection probe comprising a target-hybridizing sequence substantially corresponding to the nucleotide sequence of
SEQ ID NO:9 if the V. vulnificus-specific amplification oligomer set comprises first and second V. vulnificus-specific amplification oligomers respectively comprising target-hybridizing sequences as shown in (i) SEQ ID NO:66 and SEQ ID NO:62, including from 0 to 16 nucleotide analogs, (ii) SEQ ID NO:42 and SEQ ID NO:37, including from 0 to 16 nucleotide analogs, (iii) SEQ ID NO:55 and SEQ ID NO:37, including from 0 to 16 nucleotide analogs, (iv) SEQ ID NO:42 and SEQ ID NO:84, including from 0 to 16 nucleotide analogs, (v) SEQ ID NO:42 and SEQ ID NO:47, including from 0 to 16 nucleotide analogs, (vi) SEQ ID NO:55 and SEQ ID NO:84, including from 0 to 16 nucleotide analogs, (vii) SEQ ID NO:55 and SEQ ID NO:47, including from 0 to 16 nucleotide analogs, or (viii) SEQ ID NO:55 and SEQ ID NO:5, including from 0 to 16 nucleotide analogs; or
SEQ ID NO:31 if the V. vulnificus-specific amplification oligomer set comprises first and second V vulnificus-specific amplification oligomers respectively comprising target-hybridizing sequences as shown in (i) SEQ ID NO:58 and SEQ ID NO:21, including from 0 to 16 nucleotide analogs, (ii) SEQ ID NO:58 and SEQ ID NO:20, including from 0 to 16 nucleotide analogs, (iii) or (ii) SEQ ID NO:43 and SEQ ID NO:78, including from 0 to 16 nucleotide analogs.

15. (canceled)

16. The composition or kit of any one of claims claim 2, wherein the set of oligonucleotides comprises the first and second V. cholerae-specific amplification oligomers.

17. (canceled)

18. The composition or kit of claim 16, wherein the set of oligonucleotides further comprises a V. cholerae-specific detection probe comprising a target-hybridizing sequence substantially corresponding to the nucleotide sequence of
SEQ ID NO: 12 if the V. cholerae-specific amplification oligomer set comprises first and second V. cholerae-specific amplification oligomers respectively comprising target-hybridizing sequences as shown in SEQ ID NO:56 and SEQ ID NO: 11, including from 0 to 16 nucleotide analogs;
SEQ ID NO:35 or SEQ ID NO:88 if the V. cholerae-specific amplification oligomer set comprises first and second V. cholerae-specific amplification oligomers respectively comprising target-hybridizing sequences as shown in (i) SEQ ID NO:32 and SEQ ID NO:3, including from 0 to 16 nucleotide analogs, (ii) SEQ ID NO:57 and SEQ ID NO:64, including from 0 to 16 nucleotide analogs, (iii) SEQ ID NO:32 and SEQ ID NO: 105, including from 0 to 16 nucleotide analogs, (iv) SEQ ID NO:92 and SEQ ID NO: 3, including from 0 to 16 nucleotide analogs; (v) SEQ ID NO:92 and SEQ ID NO: 105, including from 0 to 16 nucleotide analogs, (vi) SEQ ID NO:85 and SEQ ID NO:3, including from 0 to 16 nucleotide analogs, or (vii) SEQ ID NO:85 and SEQ ID NO: 105, including from 0 to 16 nucleotide analogs; or
SEQ ID NO:67 or SEQ ID NO:82 if the V. cholerae-specific amplification oligomer set comprises first and second V. cholerae-specific amplification oligomers respectively comprising target-hybridizing sequences as shown in SEQ ID NO:94 and SEQ ID NO: 19, including from 0 to 16 nucleotide analogs.

19. (canceled)

20. The composition or kit of claim 2, wherein the set of oligonucleotides comprises the STEC-specific amplification oligomer set.

21. (canceled)

22. The composition or kit of claim 20, wherein the set of oligonucleotides further comprises a STEC-specific detection probe comprising a target-hybridizing sequence substantially corresponding to the nucleotide sequence of
SEQ ID NO:73, SEQ ID NO:74, SEQ ID NO:7, or SEQ ID NO:59 if the STEC-specific amplification oligomer set comprises first and second STEC-specific amplification oligomers respectively comprising target-hybridizing sequences as shown in (i) SEQ ID NO:70 and SEQ ID NO:16, including from 0 to 16 nucleotide analogs, (ii) SEQ ID NO:71 and SEQ ID NO:44, including from 0 to 16 nucleotide analogs, (iii) SEQ ID NO:71 and SEQ ID NO: 16, including from 0 to 16 nucleotide analogs, (iv) SEQ ID NO: 70 and SEQ ID NO:44, including from 0 to 16 nucleotide analogs, (v) SEQ ID NO:70 and SEQ ID NO: 107, including from 0 to 16 nucleotide analogs, (vi) SEQ ID NO:70 and SEQ ID NO:87, including from 0 to 16 nucleotide analogs, (vii) SEQ ID NO:71 and SEQ ID NO: 107, including from 0 to 16 nucleotide analogs, or (viii) SEQ ID NO:71 and SEQ ID NO:87, including from 0 to 16 nucleotide analogs;
SEQ ID NO:96 or SEQ ID NO: 103 if the STEC-specific amplification oligomer set comprises first and second STEC-specific amplification oligomers respectively comprising target-hybridizing sequences as shown in SEQ ID NO:90 and SEQ ID NO:100, including from 0 to 16 nucleotide analogs;
SEQ ID NO:99 or SEQ ID NO: 104 if the STEC-specific amplification oligomer set comprises first and second STEC-specific amplification oligomers respectively comprising target-hybridizing sequences as shown in SEQ ID NO:93 and SEQ ID NO: 101, including from 0 to 16 nucleotide analogs.

23. (canceled)

24. The composition or kit of claim 2, wherein the set of oligonucleotides comprises the Plesiomonas-specific amplification oligomer set.

25. (canceled)

26. The composition or kit of claim 1, wherein the set of oligonucleotides further comprises a Plesiomonas-specific detection probe comprising a target-hybridizing sequence substantially corresponding to the nucleotide sequence of
SEQ ID NO:48 if the Plesiomonas-specific amplification oligomer set comprises first and second Plesiomonas-specific amplification oligomers respectively comprising target-hybridizing sequences as shown in (i) SEQ ID NO:2 and SEQ ID NO:49, including from 0 to 16 nucleotide analogs or (ii) SEQ ID NO: 17 and SEQ ID NO:98, including from 0 to 16 nucleotide analogs;
SEQ ID NO:80, SEQ ID NO:72, or SEQ ID NO:61 if the Plesiomonas-specific amplification oligomer set comprises first and second Plesiomonas-specific amplification oligomers respectively comprising target-hybridizing sequences as shown in_(i) SEQ ID NO:95 and SEQ ID NO: 1, including from 0 to 16 nucleotide analogs, (ii) SEQ ID NO: 17 and SEQ ID NO: 1, including from 0 to 16 nucleotide analogs, (iii) SEQ ID NO:95 and SEQ ID NO: 106, including from 0 to 16 nucleotide analogs, (iv) SEQ ID NO: 17 and SEQ ID NO: 106, including from 0 to 16 nucleotide analogs, (v) SEQ ID NO: 17 and SEQ ID NO:98, including from 0 to 16 nucleotide analogs, or (vi) SEQ ID NO:95 and SEQ ID NO:98, including from 0 to 16 nucleotide analogs; or
SEQ ID NO:8 if the Plesiomonas-specific amplification oligomer set comprises first and second Plesiomonas-specific amplification oligomers respectively comprising target-hybridizing sequences as shown in (i) SEQ ID NO:79 and SEQ ID NO:81, including from 0 to 16 nucleotide analogs or (ii) SEQ ID NO:79 and SEQ ID NO:68, including from 0 to 16 nucleotide analogs.

27-29. (canceled)

30. The composition or kit of claim 2, wherein the set of oligonucleotides comprises the Yersinia-specific amplification oligomer set, the Vibrio-specific amplification oligomer set, the STEC-specific amplification oligomer set, and the Plesiomonas-specific amplification oligomer set.

31. The composition or kit of any one of claim 5, wherein the detection probe comprises
(i) a detectable label, optionally wherein the detectable label is a fluorescent or chemiluminescent label, and/or
(ii) a blocking moiety at or near the 3′ terminus, optionally wherein the blocking moiety is an inverted nucleotide.

32-37. (canceled)

38. A reaction mixture for determining the presence or absence of at least one enteric pathogen in a sample, said reaction mixture comprising a set of oligonucleotides as specified in claim 2.

39. (canceled)

40. (canceled)

41. A method for determining the presence or absence of at least one enteric pathogen in a sample, wherein the at least one enteric pathogen is selected from the group consisting of Yersinia enterocolitica, Vibrio spp., Shiga Toxin Expressing E. coli (STEC) O157, and Plesiomonas shigelloides, the method comprising:
(1) contacting a sample, said sample suspected of containing the at least one enteric pathogen, with an oligomer combination capable of amplifying a target region of Yersinia enterocolitica, Vibrio spp., STEC O157, and/or Plesiomonas shigelloides target nucleic acid, said oligomer combination comprising
(a) a Yersinia-specific amplification oligomer set capable of amplifying a target region of a Yersinia enterocolitica target nucleic acid, wherein the Yersinia-specific amplification oligomer set comprises first and second Yersinia-specific amplification oligomers respectively comprising target-hybridizing sequences as shown in (i) SEQ ID NO:26 and SEQ ID NO:46, including from 0 to 16 nucleotide analogs, (ii) SEQ ID NO:97 and SEQ ID NO: 4, including from 0 to 16 nucleotide analogs, (iii) SEQ ID NO: 102 and SEQ ID NO: 24, including from 0 to 16 nucleotide analogs, (iv) SEQ ID NO:6 and SEQ ID NO:24, including from 0 to 16 nucleotide analogs, (v) SEQ ID NO: 13 and SEQ ID NO:22, including from 0 to 16 nucleotide analogs, (vi) SEQ ID NO:77 and SEQ ID NO:22, including from 0 to 16 nucleotide analogs, (vii) SEQ ID NO:77 and SEQ ID NO:41, including from 0 to 16 nucleotide analogs, (viii) SEQ ID NO:28 and SEQ ID NO:30, including from 0 to 16 nucleotide analogs, (ix) SEQ ID NO:60 and SEQ ID NO:45, including from 0 to 16 nucleotide analogs, (x) SEQ ID NO:65 and SEQ ID NO:54, including from 0 to 16 nucleotide analogs, or (xi) SEQ ID NO: 10 and SEQ ID NO:4, including from 0 to 16 nucleotide analogs; and/or
(b) a Vibrio-specific amplification oligomer set capable of amplifying a target region of a Vibrio spp. target nucleic acid, wherein the Vibrio spp. is one or more of V. parahaemolyticus, V. vulnifcus, and V. cholerae, and wherein the Vibrio-specific amplification oligomer set comprises
(b-1) first and second V. parahaemolyticus-specific amplification oligomers respectively comprising target-hybridizing sequences as shown in (i) SEQ ID NO:51 and SEQ ID NO:76, including from 0 to 16 nucleotide analogs, (ii) SEQ ID NO:58 and SEQ ID NO:21, including from 0 to 16 nucleotide analogs, (iii) SEQ ID NO:58 and SEQ ID NO:86, including from 0 to 16 nucleotide analogs, (iv) SEQ ID NO:53 and SEQ ID NO:75, including from 0 to 16 nucleotide analogs, (v) SEQ ID NO:58 and SEQ ID NO:20, including from 0 to 16 nucleotide analogs, (vi) SEQ ID NO:52 and SEQ ID NO:75, including from 0 to 16 nucleotide analogs, (vii) SEQ ID NO:51 and SEQ ID NO:75, including from 0 to 16 nucleotide analogs, (viii) SEQ ID NO:52 and SEQ ID NO:76, including from 0 to 16 nucleotide analogs, or (ix) SEQ ID NO:53 and SEQ ID NO:76, including from 0 to 16 nucleotide analogs; and/or
(b-2) first and second V. vulnificus-specific amplification oligomers respectively comprising target-hybridizing sequences as shown in (i) SEQ ID NO:66 and SEQ ID NO:62, including from 0 to 16 nucleotide analogs, (ii) SEQ ID NO:58 and SEQ ID NO:21, including from 0 to 16 nucleotide analogs, (iii) SEQ ID NO:42 and SEQ ID NO:37, including from 0 to 16 nucleotide analogs, (iv) SEQ ID NO:55 and SEQ ID NO:37, including from 0 to 16 nucleotide analogs, (v) SEQ ID NO:43 and SEQ ID NO:78, including from 0 to 16 nucleotide analogs, (vi) SEQ ID NO:58 and SEQ ID NO:20, including from 0 to 16 nucleotide analogs, (vii) SEQ ID NO:42 and SEQ ID NO:84, including from 0 to 16 nucleotide analogs, (viii) SEQ ID NO:42 and SEQ ID NO:47, including from 0 to 16 nucleotide analogs, (ix) SEQ ID NO:55 and SEQ ID NO:84, including from 0 to 16 nucleotide analogs, (x) SEQ ID NO:55 and SEQ ID NO:47, including from 0 to 16 nucleotide analogs, or (xi) SEQ ID NO:55 and SEQ ID NO:5, including from 0 to 16 nucleotide analogs; and/or
(b-3) first and second V. cholerae-specific amplification oligomers respectively comprising target-hybridizing sequences as shown in (i) SEQ ID NO:56 and SEQ ID NO: 11, including from 0 to 16 nucleotide analogs, (ii) SEQ ID NO:32 and SEQ ID NO:3, including from 0 to 16 nucleotide analogs, (iii) SEQ ID NO:57 and SEQ ID NO:64, including from 0 to 16 nucleotide analogs, (iv) SEQ ID NO:94 and SEQ ID NO: 19, including from 0 to 16 nucleotide analogs, (v) SEQ ID NO:32 and SEQ ID NO: 105, including from 0 to 16 nucleotide analogs, (vi) SEQ ID NO:92 and SEQ ID NO:3, including from 0 to 16 nucleotide analogs; (vii) SEQ ID NO:92 and SEQ ID NO: 105, including from 0 to 16 nucleotide analogs, (viii) SEQ ID NO:85 and SEQ ID NO:3, including from 0 to 16 nucleotide analogs, or (ix) SEQ ID NO:85 and SEQ ID NO: 105, including from 0 to 16 nucleotide analogs; and/or
(c) a STEC-specific amplification oligomer set capable of amplifying a target region of a STEC O157 target nucleic acid, wherein the STEC-specific amplification oligomer set comprises first and second STEC-specific amplification oligomers respectively comprising target-hybridizing sequences as shown in (i) SEQ ID NO:70 and SEQ ID NO: 16, including from 0 to 16 nucleotide analogs, (ii) SEQ ID NO:93 and SEQ ID NO:101, including from 0 to 16 nucleotide analogs, (iii) SEQ ID NO:71 and SEQ ID NO:44, including from 0 to 16 nucleotide analogs, (iv) SEQ ID NO:90 and SEQ ID NO: 100, including from 0 to 16 nucleotide analogs, (v) SEQ ID NO:71 and SEQ ID NO: 16, including from 0 to 16 nucleotide analogs, (vi) SEQ ID NO:70 and SEQ ID NO:44, including from 0 to 16 nucleotide analogs, (vii) SEQ ID NO:70 and SEQ ID NO: 107, including from 0 to 16 nucleotide analogs, (viii) SEQ ID NO:70 and SEQ ID NO:87, including from 0 to 16 nucleotide analogs, (ix) SEQ ID NO:71 and SEQ ID NO: 107, including from 0 to 16 nucleotide analogs, or (x) SEQ ID NO:71 and SEQ ID NO:87, including from 0 to 16 nucleotide analogs; and/or
(d) a Plesiomonas-specific amplification oligomer set capable of amplifying a target region of a Plesiomonas shigelloides target nucleic acid, wherein the Plesiomonas-specific amplification oligomer set comprises first and second Plesiomonas-specific amplification oligomers respectively comprising target-hybridizing sequences as shown in (i) SEQ ID NO:2 and SEQ ID NO:49, including from 0 to 16 nucleotide analogs, (ii) SEQ ID NO:95 and SEQ ID NO: 1, including from 0 to 16 nucleotide analogs, (iii) SEQ ID NO:79 and SEQ ID NO:81, including from 0 to 16 nucleotide analogs, (iv) SEQ ID NO: 17 and SEQ ID NO: 1, including from 0 to 16 nucleotide analogs, (v) SEQ ID NO:95 and SEQ ID NO: 106, including from 0 to 16 nucleotide analogs, (vi) SEQ ID NO: 17 and SEQ ID NO: 106, including from 0 to 16 nucleotide analogs, (vii) SEQ ID NO:79 and SEQ ID NO:68, including from 0 to 16 nucleotide analogs, (viii) SEQ ID NO: 17 and SEQ ID NO:98, including from 0 to 16 nucleotide analogs, or (ix) SEQ ID NO:95 and SEQ ID NO:98, including from 0 to 16 nucleotide analogs;
(2) performing an in vitro nucleic acid amplification reaction, wherein any Yersinia enterocolitica, Vibrio spp., STEC O157, and/or Plesiomonas shigelloides target nucleic acid present in the sample is used as a template for generating one or more amplification products corresponding to the Yersinia enterocolitica, Vibrio spp., STEC O157, and/or Plesiomonas shigelloides target regions; and
(3) detecting the presence or absence of the one or more amplification products, thereby determining the presence or absence of the at least one enteric pathogen in the sample.

42. The method of claim 41, wherein
if the sample is contacted with the Yersinia-specific amplification oligomer set and any Yersinia enterocolitica target nucleic acid present in the sample is used as a template for generating a Yersinia enterocolitica amplification product corresponding to the Yersinia enterocolitica target region, then the detecting step (3) comprises contacting the in vitro nucleic acid amplification reaction with a Yersinia-specific detection probe configured to specifically hybridize to the Yersinia enterocolitica amplification product; and/or;
if the sample is contacted with the Vibrio-specific amplification oligomer set comprising the first and second V. parahaemolyticus-specific amplification oligomers and any V. parahaemolyticus target nucleic acid present in the sample is used as a template for generating a V. parahaemolyticus amplification product corresponding to the V. parahaemolyticus target region, then the detecting step (3) comprises contacting the in vitro nucleic acid amplification reaction with a V. parahaemolyticus-specific detection probe configured to specifically hybridize to the V. parahaemolyticus amplification product; and/or
if the sample is contacted with the Vibrio-specific amplification oligomer set comprising the first and second V. vulnificus-specific amplification oligomers and any V. vulnificus target nucleic acid present in the sample is used as a template for generating a V. vulnificus amplification product corresponding to the V vulnificus target region, then the detecting step (3) comprises contacting the in vitro nucleic acid amplification reaction with a V. vulnificus-specific detection probe configured to specifically hybridize to the V. vulnificus amplification product; and/or
if the sample is contacted with the Vibrio-specific amplification oligomer set comprising the first and second V. cholerae-specific amplification oligomers and any V. cholerae target nucleic acid present in the sample is used as a template for generating a V. cholerae amplification product corresponding to the V. cholerae target region, then the detecting step (3) comprises contacting the in vitro nucleic acid amplification reaction with a V. cholerae-specific detection probe configured to specifically hybridize to the V. cholerae amplification product; and/or
if the sample is contacted with the STEC-specific amplification oligomer set and any STEC O157 target nucleic acid present in the sample is used as a template for generating a STEC O157 amplification product corresponding to the STEC O157 target region; then the detecting step (3) comprises contacting the in vitro nucleic acid amplification reaction with a STEC-specific detection probe configured to specifically hybridize to the STEC O157 amplification product; and/or
if the sample is contacted with the Plesiomonas-specific amplification oligomer set and any Plesiomonas shigelloides target nucleic acid present in the sample is used as a template for generating a Plesiomonas shigelloides amplification product corresponding to the Plesiomonas shigelloides target region, then the detecting step (3) comprises contacting the in vitro nucleic acid amplification reaction with a Plesiomonas-specific detection probe configured to specifically hybridize to the Plesiomonas shigelloides amplification product.

43. (canceled)

44. (canceled)

45. The method of claim 41, wherein the method is a multiplex method for detecting the presence or absence of each of Yersinia enterocolitica, Vibrio spp., Shiga Toxin Expressing E. coli (STEC) O157, and Plesiomonas shigelloides.

46. (canceled)

47. A method for synthesizing a pair of oligonucleotides, comprising synthesizing a first oligonucleotide and synthesizing a second oligonucleotide,
wherein each of the synthesizing the first oligonucleotide and the synthesizing the second oligonucleotide comprises the steps of:
(a) obtaining a solid support comprising at least one nucleobase residue, wherein the at least one nucleobase residue is covalently bound at a 3′ position to the solid support;
(b) coupling a 5′ position of the nucleobase residue furthest from the solid support to a 3′ position of another nucleobase residue;
(c) repeating step (b) at least 16 additional times, thereby generating at least 18 contiguous nucleobase residues coupled to the solid support; and
(d) cleaving the at least 18 contiguous nucleobase residues generated in step (c), thereby obtaining the oligonucleotide, and
wherein the first oligonucleotide and the second oligonucleotide respectively comprise the nucleotide sequences of any one of
SEQ ID NO:26 and SEQ ID NO:46, including from 0 to 16 nucleotide analogs;
SEQ ID NO:97 and SEQ ID NO:4, including from 0 to 16 nucleotide analogs;
SEQ ID NO: 102 and SEQ ID NO:24, including from 0 to 16 nucleotide analogs;
SEQ ID NO:6 and SEQ ID NO:24, including from 0 to 16 nucleotide analogs;
SEQ ID NO: 13 and SEQ ID NO:22, including from 0 to 16 nucleotide analogs;
SEQ ID NO:77 and SEQ ID NO:22, including from 0 to 16 nucleotide analogs;
SEQ ID NO:77 and SEQ ID NO:41, including from 0 to 16 nucleotide analogs;
SEQ ID NO:28 and SEQ ID NO:30, including from 0 to 16 nucleotide analogs;
SEQ ID NO:60 and SEQ ID NO:45, including from 0 to 16 nucleotide analogs;
SEQ ID NO:65 and SEQ ID NO:54, including from 0 to 16 nucleotide analogs;
SEQ ID NO: 10 and SEQ ID NO:4, including from 0 to 16 nucleotide analogs;
SEQ ID NO:51 and SEQ ID NO:76, including from 0 to 16 nucleotide analogs;
SEQ ID NO:58 and SEQ ID NO:21, including from 0 to 16 nucleotide analogs;
SEQ ID NO:58 and SEQ ID NO:86, including from 0 to 16 nucleotide analogs;
SEQ ID NO:53 and SEQ ID NO:75, including from 0 to 16 nucleotide analogs;
SEQ ID NO:58 and SEQ ID NO:20, including from 0 to 16 nucleotide analogs;
SEQ ID NO:52 and SEQ ID NO:75, including from 0 to 16 nucleotide analogs;
SEQ ID NO:51 and SEQ ID NO:75, including from 0 to 16 nucleotide analogs;
SEQ ID NO:52 and SEQ ID NO:76, including from 0 to 16 nucleotide analogs;
SEQ ID NO:53 and SEQ ID NO:76, including from 0 to 16 nucleotide analogs;
SEQ ID NO:66 and SEQ ID NO:62, including from 0 to 16 nucleotide analogs;
SEQ ID NO:58 and SEQ ID NO:21, including from 0 to 16 nucleotide analogs;
SEQ ID NO:42 and SEQ ID NO:37, including from 0 to 16 nucleotide analogs;
SEQ ID NO:55 and SEQ ID NO:37, including from 0 to 16 nucleotide analogs;
SEQ ID NO:43 and SEQ ID NO:78, including from 0 to 16 nucleotide analogs;
SEQ ID NO:58 and SEQ ID NO:20, including from 0 to 16 nucleotide analogs;
SEQ ID NO:42 and SEQ ID NO:84, including from 0 to 16 nucleotide analogs;
SEQ ID NO:42 and SEQ ID NO:47, including from 0 to 16 nucleotide analogs;
SEQ ID NO:55 and SEQ ID NO:84, including from 0 to 16 nucleotide analogs;
SEQ ID NO:55 and SEQ ID NO:47, including from 0 to 16 nucleotide analogs;
SEQ ID NO:55 and SEQ ID NO:5, including from 0 to 16 nucleotide analogs;
SEQ ID NO:56 and SEQ ID NO: 11, including from 0 to 16 nucleotide analogs;
SEQ ID NO:32 and SEQ ID NO:3, including from 0 to 16 nucleotide analogs;
SEQ ID NO:57 and SEQ ID NO:64, including from 0 to 16 nucleotide analogs;
SEQ ID NO:94 and SEQ ID NO: 19, including from 0 to 16 nucleotide analogs;
SEQ ID NO:32 and SEQ ID NO: 105, including from 0 to 16 nucleotide analogs;
SEQ ID NO:92 and SEQ ID NO:3, including from 0 to 16 nucleotide analogs;
SEQ ID NO:92 and SEQ ID NO: 105, including from 0 to 16 nucleotide analogs;
SEQ ID NO:85 and SEQ ID NO:3, including from 0 to 16 nucleotide analogs;
SEQ ID NO:85 and SEQ ID NO: 105, including from 0 to 16 nucleotide analogs;
SEQ ID NO:70 and SEQ ID NO: 16, including from 0 to 16 nucleotide analogs;
SEQ ID NO:93 and SEQ ID NO:101, including from 0 to 16 nucleotide analogs;
SEQ ID NO:71 and SEQ ID NO:44, including from 0 to 16 nucleotide analogs;
SEQ ID NO:90 and SEQ ID NO: 100, including from 0 to 16 nucleotide analogs;
SEQ ID NO:71 and SEQ ID NO: 16, including from 0 to 16 nucleotide analogs;
SEQ ID NO:70 and SEQ ID NO:44, including from 0 to 16 nucleotide analogs;
SEQ ID NO:70 and SEQ ID NO: 107, including from 0 to 16 nucleotide analogs;
SEQ ID NO:70 and SEQ ID NO:87, including from 0 to 16 nucleotide analogs;
SEQ ID NO:71 and SEQ ID NO: 107, including from 0 to 16 nucleotide analogs;
SEQ ID NO:71 and SEQ ID NO:87, including from 0 to 16 nucleotide analogs;
SEQ ID NO:2 and SEQ ID NO:49, including from 0 to 16 nucleotide analogs;
SEQ ID NO:95 and SEQ ID NO: 1, including from 0 to 16 nucleotide analogs;
SEQ ID NO:79 and SEQ ID NO:81, including from 0 to 16 nucleotide analogs;
SEQ ID NO: 17 and SEQ ID NO: 1, including from 0 to 16 nucleotide analogs;
SEQ ID NO:95 and SEQ ID NO: 106, including from 0 to 16 nucleotide analogs;
SEQ ID NO: 17 and SEQ ID NO: 106, including from 0 to 16 nucleotide analogs;
SEQ ID NO:79 and SEQ ID NO:68, including from 0 to 16 nucleotide analogs;
SEQ ID NO: 17 and SEQ ID NO:98, including from 0 to 16 nucleotide analogs; or
SEQ ID NO:95 and SEQ ID NO:98, including from 0 to 16 nucleotide analogs.
